SEQUENCE LISTING (9 sequences)

SEQ ID NO: 1 - DNA, 17419 bp, Drosophila melanogaster
caaaaacggttaaacaacactagcgcaagatggctgctctgctctcgtgtgaattttttgtttaaattaaaatcgcccgtggtgcggactgtttgagcaatcgaattttattggcacgccatgcgtgggatgaactaagacggatgcaagtgcggatatgcatcagcatcaacggaggagggccgaaaatacggagaatacaatcgaaaattccacaaaaccaacagccaggtgtcggtgtgtgtatgcgtgagagtgcttgtgcaagtgtatgtgtgtatttatattcttcatatcggatcggatcgtatcggaaaggtggtgcaaatgtttataaatatatattaaggaacaccttactatatatatgcgtgataaatccaatgaaatgtgcatgtggctgatggcttaaagcgccagcacaggcatcggagtgggcagcgacgtcggcagcgcaccagcgtcggattggaatgttaactaagagcaataacaacaacatcaacaaccactgaagcaacggcagcaccaccagctacatcaaaaacaaaccaaaacatacaataaaaacaaatagaaacagcaatagctgcaaacatcattgaaaattgggtaagtccaggcatatgcatggttttgggactacttgtttttgagcccgttttaaggactccgcccaacgccctttttttgcagcgtcgcgctgtctgcttccttaatacacttgtcgttgtgttagtgcgtgtgtgcgcgtgtgtcggtgtgaaagtaaggagagcttgcgaaaaagggatagcgcaattgggcaaataaaaaagtggccaaataaaaactctgcaaacattttttgcacaattccatcaatgaaaagcaaatattgcataaacatttgcctcattatgaggatgagatgtataaatgcagatgaaatggacaacgacaaacaaaatacttttaaaagcgcacagtggtcacacgatacagtggccgcaaaaggaaatacaagacttttgattgtttacacaaagaccgcttctgttgaaatcattaaaaatatcctaaggcaaaaaacattttccccttaaaagtgtgcacaaaagtctgcattacgagttactactgtatttgttttgactatttttgggtcgaccatttgcagcttctttatgtttgtattttgatgctgttgtctgaggtcaactagttggcttcaaaacgaaagcaaaatccgaaaatatcgtaataaaaaaaatcacaaatactacgcattttggttcgttccaatcaaagcgaaaacaaaaacacgttttacaactttgcggcgcgaaattcattgttgttgtgttcgttttttatttttattttttgaataacaaacacatgcacaggcacacgcacacgcacacacgaagttatatacagaaatgctcatatatagatatagccaacaatcgggccatgaatgaccgaagcgatgcgtacaacaaaaccgatagccacagtagtaattaaaggcaataaccgaagcgtggcgcatcgtagtttttgttacgggtttccgttcatagtaatgccttttgttgttgctgtctttcggtcagtttctgcgtcgtcggcaaaaattttcggtcgctgacgtcacacctaggcgccacaaagctccccccactacccactgtcatcccccccttctccctctctctctctctctctctctctctctccggggttctatgtatcccgtttgtttgtgtattgcaacatgcgtgggcgttatactccaactgtatcggtgtgagtgagcaagtttggctgtgtgaggtcagtgttgcgtgaacttttcgcattggaatgatgatgtactaacttttggcgcccactgttcaatattccctttattgcgcctattacgactatctatctctgcccattttcacttgcgctaattacgccgttgcatgaccactgtactcgggactgattggaatgctaattggaatttgcaattttatttattccgattggtcatacgccattacttctagggctgggcaggtgatagagatagagatatatattcagttcaaattatatattagtcatatttagcgatgtctaaaataattacgtgaatgcctttctatatgcattccattgtggctgtctaatatacatataattttgacttattgaacaactgccaaaacatctgatccaatttaaaccgtacgaaaaaatacatttccctgaagaaagcctaaatccatgcaccgttcaacaatttgtttacgttgactttatcgtattaaaaaaaggctatcgaccatttaatcttatcatcaaacagctggaatatgcatttacgtttatctgggaacgcggtgcagaaatgtgtactttatgtttatctggaaccgttgatatatgtagatacattcatatgtttcgcattcttgaaattattactttcctcgttttgttgcccagcggacacattttcttcgcatattaaaggttattttttccttgccatctgtttgtttgtttatgttgcttttgcgggtatttttagcacaaatccctcgccttttgatctacttgccattcgggttcgccaggcaaattcgctgggagagctggtgcatgagcacggttccgacttgatgctaattgctccgcaaatatgcaagaaatgtaaaagagaaaaatcggagaaaatcacgaactgtgcaactgcaactgcaattggaactgcaagcgctggatgcgaaagaaacaataaacattttgatgtctacatttttgcaaacaactgattcgagagcggcaacaaattgcctggaaacggagacgaaaagtaaacatgcaagagaaaaactatttgccatacaagtcgttgggaaaaggagtgtggcggggggcacggggtttgtgcgaccaataatgggggaattaattaacatggccagcggcggcaattagaccaaataaacgaaacagaaaggaaatcgatccgagtcacaagcctccaatgttgctggatgtgtgtttttacaaagctgtgatcgttttgcctcttgaatctccgatatcacaaagtgcactgtgcgtaatgattttaatgaatttaaggtataaatatgttaggcttatgtagaaagtacaatttgatatatataatataataaattatatatgtaagtcttacaatttttctttagtttttacttgggatcgctaataatgcgattaaataaactaaatttcttttaattaagttttcccgtgtaaatataggcgaccgcagagtgggttacttttagctaggccaaaagcgagaccgcagcagctacttgaatggccaaaatcggcctaatgagttgggcaacacatgtttcgatttgacttccatagcgcgacgctgcgaaaaggagaaccgtcagtcggtggccaaatgtgcaacaggtgggcgatcgcatgcgagcgactggtcttatcgcatgcaataaatgcacgataaagagaagaagccggctcacaaaaacacgaagaaaatctgagactgagcttcgcaagcggcgctctctttctctgaaactgaaactcggaggttgattttgtgtttttttttctttcgtttcattttgtgtttttggttttatttatcgtgactcagtcgcggcggcaacttcgtgcatcgcttttcagttgaaaacttgcaggcaaggcgcgcggtcgcgtttcgattcaaaaatcgaatagtaaatagagagaatagtaaaagaaaaaataaaaataaaatcgcttgccacgcatgcgcagctcatcatcgcgtgtgtgcgaattggccaatgagtgaatcgattgcaattggctttttgcaatacataataacaataaattatgacggtatgcaaagtggcctttttttgataatccgatccgttcagcgaaaaggcacaaaaggcgaactatcgtgatgtaataccgcgcgaatgcgaattgttcatatcgctattgtaataatttaataattaattatacaggcgggagctatttttcggcttcgcctcaaggaagtgattaatgcaaaacattatcaaagtgcgaagaaagtgaaacaaaaagcgctgaaagatcagcatggcttgaacatacgtatatactcgagatttggtggcgcaatgacctttgtgtgtttgttgtttgattttatttgccatgttggggatgttcgcttaattatgggttttggccacatgtatcggacattagtatgaatctgtgtttcgagtgtgtccatgattaattcaaaattgctcaaggcatgcacggtcaaaactgaagttgattttagggcttactggtcaaagtcaggcttagctttcaattaattatgtagttagactgctacaattagtttaaatctgattaggattttcacaagaccccatatcgaatgccgaattaattaattttaaattaataaagggctgttttggttgcccaatgatttttgcacaaggtggcctttaattgatctgatcacctttctaaaatcattcaaattcaagctcttcgccaaaccatgtaattttttcgtgtgcaactaatgaagacgatcgcgtcgcggctgcatatttgattgtttgaaagtctcgaatgaaagtgccttggcagcatgttactgtactgtacacttccctcccctcaccgattaccacttttttttttttttttttttttttttttgtttgcttcgggccaacttcaatcacagagtgctaattgttttctcaggtctccgtggattgaattcccccagtcgacaagttcccattactgcaatttcttcgcttattctgccattctgctcgctctctaactgcactttttaatccgatcccaaatcgcagatcgcagatgcaactgcggcggatcgagcgcagctgatctcgaaattgcatgaatcactcggcctaaaagaccctctgcacaatcgatttgcacgctaattttggatttggcgacgtgcagacactcgactttgacaaaataaaaactggaaaaaaataaagactgcaaattcgaaaacattgtaggctggcgtgtcggggcattttcaattttccagcgcattagtcatcgccggtctaatcggccaacaaaaaactaaagaaaaaaaaaaaaaacgaagaaaaaagcaaaaccggagccttaaactgtaactagttttttccaacgctctctgagctgagaaactaatccgtcgtcctgcatgaatctcgcactgaaatttgtatctccaagctggctggaccaagtgaaagtggacaaaaaaagaaatatatatgtagaaatagagaaaaaaagacttcaaatacaactgcagcagacgcgtgaaacaatttctcatatttttcttggctccccgtacacttcggttatgtgttttccatccacttattcaatttgccattcactgcacacaagtgaagacgagcttaacccccaaactggccgaagatgtgcccccaaaccctccaaagccccccaaagaccacacttaacactcgaaagccaatggcgcatttcatttctcggctttggactctttcgttttcttgtaatgcgtgccaggcacgttaaagtaattgtacaaattaatttattaattaagccactggccggaatggtcagaaacaatagcgaatgctaaatgctctttgtctcaagttaaattgccgatcatttgttttttttgctccaagtcagagctgccgttttggtgaaaagaggagggtattcattcgagaaactttcactctccgcttgccaaaattatgtatgcaacaggtattgccgcaaggttttaataaaacctattcgaaacgcagctccaaaatccccaacgctcatttcgaggatatccggaccagcaaacttacgcatctcgaccaggttcaaaaacccgacccattgtctttagctcttctgttttattttggcttttctcctcgcttattgcccactttgtctcagtctcagtcaacggtttagacttttttggcctttattttgattcgactttgtggcgttggcttttcattttgcatttcgtcagcggcgacacaaactcgttgtgtcatacataattctgttattgttttatttaattagttgcgttgtctttcttgtgcggtgcgcttctttgggcgaccgaacgcttttcccactccgaaaaatagaaacactccatcgtgcggagaatttcatttggccgccaccgttcctttgtgggtcatgataagcaatcactttccacgtgcccaattctggctcatttgaatagttaattgtaagttgacatttacctaggaaagttcaagaaagtgaagatgcagtgaatgttggcaaatatttgggaattctatgataaagattaaacttaaagccaactgatatcctcttcccttctgcccctttgtatatccataagatgggcaaataattgaatcctgctgcttacaaactaaatggcaatatcagtgcagaaatatctcttttccccgccaactttcccaacatttgtcacctgtctttaggggatagacaatagttttgcttagctcactgcgcagtcgacgtcgactgcgaggttcccacgcccaaattatttgcacaatatgtagccagagctctccagtgaattccaattcaattcaggcggctggttcgtgttttcccaacccagagaaataagccaacatggaatgccgaaccagcatttggcccaaccagcatttttcgctgtttgtggagcactccaagccattgtcgactatttcagtgagccgctgacaaaccgttaatgtggcgaattgacttgctgaggaaaaagctagccaaaagccagtacaaaaaaaaacaaacatatagatggcaaagagttgttattattatttagttattcctcgcaatgaagtcaatttctgtacgtagctcggtttttttttcatcatctagttttcaaccttttccatccaggccgggcttttattccattcctctttgttttttttagcttgaattttctctttagaaattttccatttgcgctttttttttgctaacaattatctgccctggtcttttctatcgattccgcccagttgtccattgtggatggagaatcgacgtatccacatgctggccgatggcaaatcgaagcgcttacagaaccttaattagtattattagaaacgaaaacaatttgcccggctttgtccactacggactgggcattagcctcgatttcagttctacaacaggctgaaatggcgaatttttatgtttgtactatctgctactgggcacccacaatggcatccattagaaccaactcccattttgatttcactatgtgtatatattgataattagatggtaattggaaagtattggaaagaattttaactagaatttgaaaaggtcttttgtagggatcatacaattaaagttcaaccagaggtttttgcgccatcaaaatcagaacttaagttagtgcactagaaaccatttaaaaagcggactgacaacgaaagtaaacgcaatgcgggaacatggtgacaacaattaagataacctgcgatggagtgttcatccagtccaactcgtacctcatgcccacgccttctcccagcggagcagacgccacggtatcgtcatccttggaacgggtgctgaaacccgcgtccgcatccgcctctccatccaattcgaacaacgaaagtgataactcgcagacgagcagtggcagccagacggcgtccagtgccagcaagcgatccaatcgatccgtgggccggagtcatcgaggttcgattgcgcaggacacaaggcgacgctactactttggcagcaaggacatctactggtgcaagcagagcagggaggcagctgcggcggccacgctctcgggctcgggactaacgcgtagtgccagcagtgtcacccacaaggaagcgtccacccacgccacccacactggtggtcaggtaatctcccgtgcgcgtcgcaagcccatcctgctttgcagcagcctgagcgctgcctatgctcctcctcgtcggcgtgagttttacttctgcgacaagtatgcacctggtccagcaagaagtactcctactacaaccacaacatccgcaacatcggccaaacacgaatcctccccgcccatcgacgcacgtctgctgaacttcctgcggtgcgcacatcgaaagttccaggcatccaaagccaaaactgagctacagcaaaccaaagacagattcctgggaaattccagagagcggactagtaagaacgggcagcagacgtcccgctcgctgccacccattgccagttcggaggagtggttcggttagtagataacaagatctatattataacgaatccaaaaaagacgtaactacatatatcaaaccatcaacacagatcaaactatacaacttcaaatctatattctaaactgaactactctaattgattagttgctttttgtttccagaactctggtcgttctttccctttaagtatctgaaatagtgttttgtgcaagccgcacaaattcatatctcataccagtcagcaattcagccctgctcccaattgttgcggctggtgttgcaagcggtccgcagatttagtttcaagccccacgacattgggcatgggtattggattatggtctaaccgttgggcaataactggcggtgtccaattgcggctgctccaagatccgtggaggtagattcgaagaaggagaagggttcttctggtgcacaaactgcactccgcttcgctcagggtcaaccacatggggcagctaaccgtgtaattagctgatacatgtatcggtgtttctgcacaggtgtaggcttggcttccttcttcctgcttgtggttttcctgctgccgctgctgctgctcatcgccttgcatgcgatatgcaaatttctgggcaccggcgaaaaccatcacacgccattcatgagcgcctcgcgttccattcatgccatttttcccccattttcccatttcccttccccttttggcccacaatccagcgactgactgactgactaaggttaccttcctttctttcttcgggcctgcactttccaactggtggcacaacacaacacaacacaatggcccaggaaccgccggcaactgttgactgactttgtgcaggagtgattgtctgccgtgtgtatttacctgtcgttgcagccccccctttcagacctcctccaacagccaaaaagaaagaaaagcttgtgagaatgtcggaaacttggtcccctggttgctccatatacatatttctctatatgtatttgtttatgttaatgccacgccatgaggtagtgcaattattcacggaaattggcaaggcacaggtgataaaacatcgcaccttgtgttgggtttatttttgcattcgtgatctttcagttcagacattgatttttgggcacatagtttcaattagaaataaaagaaataaaggatagatacataggtgagataagttagttatcatagaaaagatacatacgagatagttataataaatgttttcactacttaaaagcgagaatttggctattatttttatgactgccgaacaaaagatatatcatccctctgaaagtatttatgttcctttgttatttttgcgaattgtatgtgttgtgcactaactcaaacaataaggaagcaatacaaaagattctcacgtatgtatgtgctcgttggcttcaaaggtgttgcactgttttcctttcctatatctccggcttacacagatacaatttcattactcactcttgccacccaagggtggaacttctgacagacagaactttcgttagccaacttcgcttgtttctggggattaggtaaactacttgggcgttactaaccacttccaactggagtagtgaaatgcgaatgaatctttatagtgcaaaaaagtgcagcacaaccagctgaggtattttacttatagattatagattgagcacacaataatgttttgtttctattgctcagtcgatgtacaatatccaaatagttctccttttcggacacattttgagcagtgcattttcgcggctttgcctaattaatattacctcatcgggacagtcggattactgcgcatttcatttgacttttgcttttgcctttgctcaacacctacaacacagtgcacagtgcattgcaaaagtattcgtacccgcacgccatgtgtgcgttgaactgccaattgccaatgaatcattcaattttattgtttccccattttttttttgttcacgaacctttggacgcgctttttgcactcatcatgtccggcaggtcggtgtcgttaatttgggctttttgcatttttatgattattccgttttgagtcttttaaacaatacacaaataccgatgtttattcagaacgagaacaattgatttatatttgaggagcgtgtttcggcacaccactccccactcttttatttaccttttttgtttacgaattcccgaggtggtttgtgttcgactggcgtctacaactcttggctagcctaaaagtaaagtgcttattaattatttttgtgttgattttgtttgagtattttgcaagaacagcgctgagctcattattaacacacggccaagccaacaggtgtgtgtgtgtgtggcaacacctgtcgatatcagcgagtacatattatgtacctgcctagcacgcacatgcaaagtcgctgatgtaaacgtcgtcaggtgcgggggaacactgtaattggcctgtcaccggcgttgactaatcaatggctccggatcactcccagcgatgacagcctgagtgggcattggctttacgtaagattatagccgggattttcacattaataccctatccggtggttgtacacttataaagagggtatttttactttcttataaagggaagtagtgatcaccatctcggtgcagtctaattggatggtttaatcaatggtagctccatagagtttcagttactaaccaagctctcaatatctgttgttccacgtaggttttcttaatgttctttatatatttgttggcataagaatacgatttcttgcttccgctgactcacacttcgaggatttactcgggataaggatgactgtttcacttatttggagtcaaggtcgcggctggagaagcaaagccctggtacattgttcaagtttaaaaaatatgccactatcggactgcggtaaaaatgatgtaacaaatgtgcaaaaagtatttttaaatatatgttcaaaagataaaatcattcatgttcacatatagcttacgaaatatattacgtaattattgtgataagtggcattaggctttttattaaaacagttaagtgttctgattctttatgttcaaaaatctgaaaagcgtttggggatttctttgttcgcagtaccaaatcgatgcgaaacccgatttcgatttacctttctcgcagcagcgctgaatttcacatttgtggccgaacaattgaaacaaaaagcggacagtacgcaaatataaagactattgtctcggggtgtgacttcaaagctccgactccgctgaacaggaagccgctgacgaatggcgatgactacgaccacgactgcatgaccgactgcaattccagattggattctcgtattcgaacagtcatgcaaaaccaatttggagcgatagccgaaacttcaggttgctgcgaatggcggatgtggctccaaataatttacgggcaaggtctgtggcattcgagagcggtaaagtgacctgcgcgtgcctcctcctccactgccgtcaacctgtcgctatttgaacaatgcccacgcctcggcttcgccccactcccacgataaattcacgtcccagaatgagaacttaatatgcaaaacgcgcttccgcccagcgagcgagattcgctaatgaaatttgcataaaattaagaaattaagcgcttgaaaagaggcttcactgcctaaaacagcgcaattacatgtgaagtaaaaaggttggaagagcatgtagcacttagcgcacaaactgattttattgcacttcatttgaaattcaaattcacaaatacagacatttacggatgaaagtgatgcgaatccttcatttgatttaagccaattttctttcaatttctttgcgtgtacagatgcgcgggtagaattaaagcgacatggaactgactcatcctaaatacatagtcgtgtccaggcacacagtagtagcccaaagcctgaaagtgcccaataaaatgggtttaattatgcccgtcacatgcgaatgctttacatttggcgagcggattcgcgcggttcacccgattaacatgccatttgtgtccatttgagtcactgaactgcgtttttgagtgtgttgaccaacaattagcattgcgcatccaagtcgccgcttatgcaatcgttgttatttgtccactgacttgactgactgattaaataaacgtgagtttggccttcatccgatcacgggaactgatttggctggttatacggcaaaaaacagctcaatcggcggtgaatgcgtctatctaaatatgggaatcagatatcaaaagtgataaatgcttatattggcaactgcaaaagggaatttaaatggatattactttgtctgatatcgatatcgtgacaatttcgatattcaaattgccaagttatgtcagaaatggagaaaatatccatataatcccattgataattgtgtgcgggtgataaaacaaattacatatttggccagctgattgctattcgagtaaataatatagttaatgcagataacattgtccaaatgtgcctgtgcaaacctgtgataagattagatgcttttttgcctcctattatcagcctataaaaataacatcatcaattgataaagctgaacactgaaaaagactaatgtttgtacacattttggttttttgtttgcagacgaggaggcacaagccaggaagactagccacgagaagagccagaagcccaagccgaagccggaggaaaaccataaggagaaggaagcgatctcgagaaaggcgtccggcgagggtgatctgatcaccgttgagatcaagcagggattgctgaggaagcagacggcaaccacgacgaccggccaggctatgaacagccacgcgactgccacgcagaacggacaaaccacggtcgtcggacaccatcgcggattctcgcagccctcggtgacggtggccgctgcagcggcaacgggcagcacggctggtgatccatggttcctgcagaccaccggtcaccagatgccacccactgcaccgttgcggcacaacaagcgtcctgccccccagccgaatgcggttttgggagcgggttcctcgggaacagcgcctggatcggcggttggcgccgtactgctccaccaacagcagctcagccagtcgcagccgcacattgtgccgcccagcataccgccgcaccaccaccaccatcgcgagaacaatgtggtatgcgaaacgggctatatttaaatatattaaattgctctactgacattgctctccattgcagcaccccatccaggcgggcgcggcagccgcattgcatttgtcatcgcatgcgcttaatagtcataatttaatcagcagcgccagcaatcattcgcccaagtcccccagccagacgcagcagcagcaacagacgacgaaccagcagcaacaacagcagcagcagcaggcgtcccacagcatactgtccacgtttgcgcccgcccccactgtcgccgccgttcagccggcgggatcgcagtccgttgccggtaccgctgccgcagtggccgccgttgcccagcagcagcaacagcagcatcttcagctgctggccaactgtccgcccggtggagcgggtctaatgtcctcctccctgaatgcagcccaaatgggcatgcatggcgccgagcgagcgctgccgccaaagagtctggcgctgagtggcagccagcacggcagcaatatgctactgcacacagcaccgcaaccgccacaggcatcccagccagcggcagtcgtcggcgtcagtggtagcggatcggcgggtatgtccacctcgctgcacagcttcgatattaatggagtcacggcaggcagcggggtgaatgcgagcagcgcgtggtcaagcggatccaccactgccatgaaccatgcttccctctcgcccacagcgttggcaccgcaacaaggtcggtccaggtgcgaggtcaaactgaacgccatgccgtaagttaatatgccacgctgaacgtacagattataagcaatgtgcttcttccttagatggttccatggcagcataacgcgggatgaggcggagcatctgctgcagccgcgtgaggatggtttgttcctggttcgcgagtccacaaacttccccggcgactacacgctctgcgtttgtttccagtccaaggtggagcactaccgggtcaagtacctagagaacaagctgaccatcgacgacgaggaatactttgagaatttgggccagctagtggcggtaggtatcaccagaagacatctacgaaccatatttaaccagttcatacttgcagcactacgaggcggatgcggatggactgtgcacccagctgattaaatgcttgccaaagctgggcaaacaagagttttgcattaactccaaagatttcgtggacaagggctgggtgatccctgaagcggagctgcagctgcgcgagagcatcggcaagggcgagttcggtgacgtgatgctgggcatattacgcaacgagaaggtggccgtcaagatgctgaaggacgagggcgccgtgcagaagtttctggctgaggcctcggtcatgacgtaagttaatccacagatcattcatttgattcacagatctcaccaactccaactttaaatgcttctttagcacgctggagcacgacaatttggtaaagtttatcggccttgtcttcaccagcaagcatctgtatctggttacggaatatatgagtaagggatcactggttgattacctgcgatcgcgaggacgacagcacataaccaaaaaggatcaaatcatttttgcctagtaagcaatattgttagcttttgttttgaatccttactaattaacgttactcaatagcgacactgcctctggtatggagtatctggaagccaaaaaggttgtccatcgcgatctggcagctcgcaatgtccttatatcagaggattgcgtggctaaagtatcggatttcggtttggcacgcgaggaatgctacaatcttgacgttggcaagctgcccataaaatggacggctcccgaggcacttaaaaatggggtaaaattaaaatgactgaccagcaaagtgttgcgcatatcaactaatgtactttattctcgtcattttagcgtttttccaacaaatcagacatgtggagctttggcatactgctatgggaaatctactcctttggacgcgtgccctatcccagaattgtaagtgcagatatcgatggcattaactttagatgtgactaaccaagttttcattgtacagccattagccgatgtggtaaagcacgttgaggtgggctacaaaatggaagcgcccgagggctgtccaccggagatttacgaaatgatgcggcaggcctgggacctaaatcccgccaagcgacccacattcgcggagctcaaggtcaaactgcagctactgaacaatgccacggcgtgaggagtcagcaagggagtgacgccagtgacgcgcgatagggatgaactaaagcgaattgtttagaccagatgtcatttaagccatgggcagagccaatccctgccagaaatgggccagatccaaagctgaaactgacttgacactgaacgcaacaatttttcgtacgattatatacatacatgaatatataactactttacaacaatttatttacatatataatttatgaagtaagttttgtaagcctaagcgaaggaacccaacacaatcaagcaatgttagtgagttgcgccagcttccgacatataattaattgataaccaatcgaacggactacgtgagaaggagaggagaagggcagctgcgtagcctacctactactactacctttaactacctaccacaatattatgttatgtgtacacttaaccatagctatttattagttaacttctcggttcggactccactgtaacatttaacttctggattcgtgaccacgtctgtatctttatgttatagaatacgcatacatatgagcgccttccgttcggagggcgcctgtctaaagcaattgtttataggatgtttccctttgatatcgtaaaaacgacaagttttattcgagttccgtatactctttctgttaaccgtttctgtttgtagcacaaaactgagggatgaccacaaagaacttgtccaacaagtcgttggcaaagagatcacgctgagaattagttaaatttcgttacatttatttgtattaaaagaataaaaataaaacgaaaacaaagccagcaaaaattatgcaacatgggcaagcgaaacctaagcataaataaacgaaggcggctgataggctgcgtgtccttaaactcaagaaaacgaaaaagagaaaaacacttgtgaaaatatgcattacattaaaacgtctacatatacataatacaatacacattacctacgtatttagcttataaatcgattaataatatacaaatataaattattacgcgcaaagttcgtggatttgttcattattcggtccattcttctaacaaggaatctccttctctgtactttaatcgaacccatccaatgcatctggcggcaaggcaaaggaaaattgtaatttattttttaaaaacctaccccctaattaacttaacaaataatgtctattttaaagttccctttaagcaaattgtacatttatgcgttttaattttcgtttaagggcagccactttgctttaacacataattttagtatactttatgtaaaacacacctaatacaccaacaacacacaccccctcacaaacacacatccacgtccacatggtatatatttaattaattttaaagttattatttttttacaaacacacttcaatccacattgtaaacgaaagttctatgcgttagtgaaaccctggagaaagcaacacaaatatgcaccaacacacaaaacttaaacgcggtgattaaaggagggcagagagttctaaaacttttaataaaagtaaagaatacaagcatg

SEQ ID NO: 2 - DNA, 5075 bp, Drosophila melanogaster
ctcgtgcaaagcggttcggttggtctttaagcgattttccacaaaccaaaaagtatttccgcggaaaagctgcccgaaaacgggaaaagagagcttacgttctgcaccaaaaacgaaaaactattaatgttcgtggtctaaatatccgcaaaactcattgttattttttcatattttctgctctggttttcgcacattaattgataaatgctacttatcaatttgtggcaaaagtaattaagcttgtgacggaatcgaacgtggctaattggatacagcattggaaaagaataccatcacagagaaaaaaaacacggaacataaaaaatcaaacaaatagcgacaatttgtagagaaaacccattaaaagcgacgttcactatgtgtaaatatgtaatgtgcgtctacgagtgagcgaaaattgccgactgataactgactttttgaaaaagtaaccacgatggagtcaactactattgtttttgtgactctgctcacaattataacccaacgtaaacactgtgcgggcaaatcccttagcctgccacaaaacatttgcattgtatttcatattttgctcacatgttggctgaatttatgagctggcttgacacatttatcagccactcaaggttggcctctgtcttctacttcgccgtcgatgtttactttcccaccacgtcggtgaaattcaatatgcccatcaatgaggaatcggagagcatattctccaaaatcccgctagcccagttccaagtgctgcggatggaggacaaccggttagccagtgattacttgtatagcctggagcagaatccactactccgaataaacagttcctccggcgagatatatatgcgcactgactaccgctcaccaaactcaagtgccacattcttggtgaccgcatttcccagagatcaaccggatcacgagctgctgaatgtttcgcatctttcgttggaggttacacctcagcccctggaggagtactgttcggaactggagcacatttgcttctggagcagtgctcagtacactatagcagagtcgcacggtccatatcggcggaaggatttttttgaacccgtacttatcggcgcccttaattcccgcgctgcgaagtatctgtgtcctcatgtatccctggaatattccctgaacgctggtagttcccattttgttttgaaacaaaatcgactctacacccgacaaaccttggatcacgacgagctcaatggactgaatgccaaggcagggcagctgcaggccaggattacctgcacggttaaattgtccagcagggatcagagaaaattctcgcgcatcttggatatcaagttactggatcgcaatgataatggacccaagttgcaggagagtagctctaagtttgatttctatctggagcagccctacttccaagcggacgaggaggcgggaaaaaaagtaatctacgtggacaaggatacattggaggcaaatgctcaccttgtctacgccgtccacaatgactctcatggtctgtttcggcccgactgccacgcctacgaggcggatcacacgggcagaccacataccatcgtcagttgtcaactgcgattctcccgaaacggtgtcttccgggaaaccccctattgtgtgtccttggaggctcgggatctgaccattgtaagccgtgtcgatgccatgtcagcgacagccaatgtttgctatcatattaatctgagtaagcttcacgaatctgagcaagaattaccgcaagctcttcccctacgggcacgtcaacatcgaatattcgagagcgaagaattcaatggagattctgcaggccgatctctaagtcctccgaccgtggattacgataaggatgtttccgtatacagatcggctgcttctaattttcgagttgtccagcctgacagttttttggacttgatgcggttacgatctattcgattcgatattgtggaggataaacttggagcttttggtattacctcaacatcgggtattgtctttgtgaagaacccacaggttttggaggaggcaccggaaaccatatacttcctgaatgtcacctggatcgatcagcaaaggctgtcgcacgtgagagtgatcaatgtgcacttggttcatggtagacccgagaatactagttgcgaactgaaggtcaagtctcgatcacagacatgtgcccagattaaataccaatcgcaatgcgttcgatattgcggcttggccacaggtggtggatcttgccagtggagggggtccaactcagccatgttcggcactagatatggttcctgtgtacccgaatctcgttactgtccagatcatgtctgtgatcccctagaggaactgaatcctatggcctgtccgcaggattgcacgccagctggaagaatcgtgggtccccattcaagtaatgagaataagagagggatatacagtgcctcgggtacctgcatttgcgaggataatggcaagtgctcgtgcgctccgttagatgaggaacccaagatgaagaaaccgcgaaaacgaaaaaacgaaacagaggcggaacctctgctgggggtacgaaggggcactcctccgaatcagccacttcaggatcccatgcttctgggtgtcctaaatgtggccggtttcgaatgcgatcgctcctgcatgttcttcgtgatcacgtgccctctattgttcgttctcctgctcctctgtttgctgattgcgcagagaaagatgctccaacgtcgcttgggcaagcaatcaatgaccacgtcgtcgaaacaagctcttccggaatcaggaggcggagatttcgccttgatgccgctgcagagtggcttcaggttcgaaagtggggatgccaaatgggagtttcccagggaaaaactgcaactagatacggtcttgggagagggtgaatttggtcaggtgctaaagggctttgccaccgagatcgctggcttgccgggaataaccacggtggccgtaaagatgctcaagaagggctccaattcagtggagtacatggccctgctttcggagtttcagctcctccaggaggtctctcacccgaatgtgatcaagctgctaggcgcctgcacctcctccgaagcacctctcctgatcatcgagtatgctcggtatggctctctgaggagctatcttcgactcagtcggaagatcgagtgtgccggcgtagatttcgcagatggagtggagcctgttaatgttaagatggtacttacctttgcttggcagatttgcaagggtatggcttacctttctgagttaaagttggttcatcgtgatttggctgctagaaatgtgctccttgcggatggcaagatatgcaaaatatcagatttcggactgactcgagatgtttacgaggacgatgcctatttaaagagatcccgagatcgtgtgcccgtcaagtggatggctccggaatctttagcggatcatgtgtataccagcaaatcggatgtgtggtcctttggcgttctctgctgggaactaatcactctgggagcctctccgtatcctggcattgctccccagaatttgtggtccttgctgaagacgggctaccgcatggacagaccagaaaactgttcggaggctgtctactctatagttcgaacttgctgggcagacgagccaaatggaagaccctcattcaagtttttagcatcggagtttgagaagctattgggaaacaatgccaagtacatagatctggaaacgaatgccgtttcgaatcccctttattgtggggatgattccgccttaataaccacggaattgggcgaaccagaatcgttgcagcacctttggtcacctcccaaaatagcctacgacatccatgaccaggccaccagctacgatcagtctgaggaggagatgccagtgacttcaacggctccgccgggttacgacttaccacgacctttgcttgatgctaccgccaacgggcaggttttgcgatacgaaaacgatttgcgattccccttaaatattcggaaatccagttgtactccaagctacagcaacatgaccagtgaacctccagcgaccacctcactgccacattattctgtccccgtgaagaggggtcgatcctacctggatatgaccaacaagagtctcatcccagacaacctggacagcagggagtttgaaaagcatctgtccaagaccatctcgttccgtttctctagtttgctgaatctcagtgaaacgaaggaggtgagtccaggatggcaagctgaggatgcagtctagaaatagtgtttcccagagttctaatctcacgcctgtttttcagttcaaactgtttgttgattagtcttagttttacatataatcaattagctttacgttgacgccaaatatggattttaagataaccgatttcgatttcaaatactcccaagcatttatacactattttaactagccaaagacagccttagttttttttattattacttcttattacttttgcttcaaaaagccatattcttaaaagataattaactttatttgtaaattgccaagttcgtcctattatatgccagaagaatgaatggttatcccaaagaatttttacactcaataaataaagtttttaaaggaatatttgtgcttcaaaaggggttttctgtatttggatatttatttcaatttaagattgtaaaactctgctaaagtaagctaggttgatttataatgtccatttaatgtaatagcagtcataatataagtttcaatttatttacgccataagctaaacaatcataagtaaccttcattattaatcattgagtacatctggtacatcttaaacaatctcacaggcactctagatatttaggtattcgaaccaatgtacttatttatcgatgacttttatgactttaatgaatgtaaatacttgcatctactgcatatgtttaaattaacttttaaaaattatttgaaataatataaaataaaataaaccaaatataacagatagcggccaattggggcatcactcttcatcgggtaccagcaagttg

SEQ ID NO: 3 - DNA, 614 bp, Drosophila melanogaster
caaaaacggttaaacaacactagcgcaagatggctgctctgctctcgtgtgaattttttgtttaaattaaaatcgcccgtggtgcggactgtttgagcaatcgaattttattggcacgccatgcgtgggatgaactaagacggatgcaagtgcggatatgcatcagcatcaacggaggagggccgaaaatacggagaatacaatcgaaaattccacaaaaccaacagccaggtgtcggtgtgtgtatgcgtgagagtgcttgtgcaagtgtatgtgtgtatttatattcttcatatcggatcggatcgtatcggaaaggtggtgcaaatgtttataaatatatattaaggaacaccttactatatatatgcgtgataaatccaatgaaatgtgcatgtggctgatggcttaaagcgccagcacaggcatcggagtgggcagcgacgtcggcagcgcaccagcgtcggattggaatgttaactaagagcaataacaacaacatcaacaaccactgaagcaacggcagcaccaccagctacatcaaaaacaaaccaaaacatacaataaaaacaaatagaaacagcaatagctgcaaacatcattgaaaattggac

SEQ ID NO: 4 - DNA, 4106 bp, Drosophila melanogaster
caaggcgcgcggtcgcgtttcgattcaaaaatcgaatagtaaatagagagaatagtaaaagaaaaaataaaaataaaatcgcttgccacgcatgcgcagctcatcatcgcgtgtgtgcgaattggccaatgagtgaatcgattgcaattggctttttgcaatacataataacaataaattatgacgacgaggaggcacaagccaggaagactagccacgagaagagccagaagcccaagccgaagccggaggaaaaccataaggagaaggaagcgatctcgagaaaggcgtccggcgagggtgatctgatcaccgttgagatcaagcagggattgctgaggaagcagacggcaaccacgacgaccggccaggctatgaacagccacgcgactgccacgcagaacggacaaaccacggtcgtcggacaccatcgcggattctcgcagccctcggtgacggtggccgctgcagcggcaacgggcagcacggctggtgatccatggttcctgcagaccaccggtcaccagatgccacccactgcaccgttgcggcacaacaagcgtcctgccccccagccgaatgcggttttgggagcgggttcctcgggaacagcgcctggatcggcggttggcgccgtactgctccaccaacagcagctcagccagtcgcagccgcacattgtgccgcccagcataccgccgcaccaccaccaccatcgcgagaacaatgtgcaccccatccaggcgggcgcggtagccgcattgcatttgtcatcgcatgcgcttaatagtcataatttaatcagcagcgccagcaatcattcgcccaagtcccccagccagacgcagcagcagcaacagacgacgaaccagcagcaacaacagcagcagcagcaggcgtcccacagcatactgtccacgtttgcgcccgcccccactgtcgccgccgttcagccggcgggatcgcagtccgttgccggtaccgctgccgcagtggccgccgttgcccagcagcagcaacagcagcatcttcagctgctggccaactgtccgcccggtggagcgggtctaatgtcctcctccctgaatgcagcccaaatgggcatgcatggcgccgagcgagcgctgccgccaaagagtctggcgctgagtggcagccagcacggcagcaatatgctactgcacacagcaccgcaaccgccacaggcatcccagccagcggcagtcgtcggcgtcagtggtagcggatcggcgggtatgtccacctcgctgcacagcttcgatattaatggagtcacggcaggcagcggggtgaatgcgagcagcgcgtggtcaagcggatccaccactgccatgaaccatgcttccctctcgcccacagcgttggcaccgcaacaaggtcggtccaggtgcgaggtcaaactgaacgccatgccatggttccatggcagcataacgcgggatgaggcggagcatctgctgcagccgcgtgaggatggtttgttcctggttcgcgagtccacaaacttccccggcgactacacgctctgcgtttgtttccagtccaaggtggagcactaccgggtcaagtacctagagaacaagctgaccatcgacgacgaggaatactttgagaatttgggccagctagtggcgcactacgaggcggatgcggatggactgtgcacccagctgattaaatgcttgccaaagctgggcaaacaagagttttgcattaactccaaagatttcgtggacaagggctgggtgatccctgaagcggagctgcagctgcgcgagagcatcggcaagggcgagttcggtgacgtgatgctgggcatattacgcaacgagaaggtggccgtcaagatgctgaaggacgagggcgccgtgcagaagtttctggctgaggcctcggtcatgaccacgctggagcacgacaatttggtaaagtttatcggccttgtcttcaccagcaagcatctgtatctggttacggaatatatgagtaagggatcactggttgattacctgcgatcgcgaggacgacagcacataaccaaaaaggatcaaatcatttttgcctacgacactgcctctggtatggagtatctggaagccaaaaaggttgtccatcgcgatctggcagctcgcaatgtccttatatcagaggattgcgtggctaaagtatcggatttcggtttggcacgcgaggaatgctacaatcttgacgttggcaagctgcccataaaatggacggctcccgaggcacttaaaaatgggcgtttttccaacaaatcagacatgtggagctttggcatactgctatgggaaatctactcctttggacgcgtgccctatcccagaattccattagccgatgtggtaaagcacgttgaggtgggctacaaaatggaagcgcccgagggctgtccaccggagatttacgaaatgatgcggcaggcctgggacctaaatcccgccaagcgacccacattcgcggagctcaaggtcaaactgcagctactgaacaatgccacggcgtgaggagtcagcaagggagtgacgccagtgacgcgcgatagggatgaactaaagcgaattgtttagaccagatgtcatttaagccatgggcagagccaatccctgccagaaatgggccagatccaaagctgaaactgacttgacactgaacgcaacaatttttcgtacgattatatacatacatgaatatataactactttacaacaatttatttacatatataatttatgaagtaagttttgtaagcctaagcgaaggaacccaacacaatcaagcaatgttagtgagttgcgccagcttccgacatataattaattgataaccaatcgaacggactacgtgagaaggagaggagaagggcagctgcgtagcctacctactactactacctttaactacctaccacaatattatgttatgtgtacacttaaccatagctatttattagttaacttctcggttcggactccactgtaacatttaacttctggattcgtgaccacgtctgtatctttatgttatagaatacgcatacatatgagcgccttccgttcggagggcgcctgtctaaagcaattgtttataggatgtttccctttgatatcgtaaaaacgacaagttttattcgagttccgtatactctttctgttaaccgtttctgtttgtagcacaaaactgagggatgaccacaaagaacttgtccaacaagtcgttggcaaagagatcacgctgagaattagttaaatttcgttacatttatttgtattaaaagaataaaaataaaacgaaaacaaagccagcaaaaattatgcaacatgggcaagcgaaacctaagcataaataaacgaaggcggctgataggctgcgtgtccttaaactcaagaaaacgaaaaagagaaaaacacttgtgaaaatatgcattacattaaaacgtctacatatacataatacaatacacattacctacgtatttagcttataaatcgattaataatatacaaatataaattattacgcgcaaagttcgtggatttgttcattattcggtccattcttctaacaaggaatctccttctctgtactttaatcgaacccatccaatgcatctggcggcaaggcaaaggaaaattgtaatttattttttaaaaacctaccccctaattaacttaacaaataatgtctattttaaagttccctttaagcaaattgtacatttatgcgttttaattttcgtttaagggcagccactttgctttaacacataattttagtatactttatgtaaaacacacctaatacaccaacaacacacaccccctcacaaacacacatccacgtccacatggtatatatttaattaattttaaagttattatttttttacaaacacacttcaatccacattgtaaacgaaagttctatgcgttagtgaaaccctggagaaagcaacacaaatatgcaccaacacacaaaacttaaacgcggtgattaaaggagggcagagagttctaaaacttttaataaaagtaaagaatacaagcatgaaaaaaaaaaaaaaaaaa

SEQ ID NO: 5 - DNA, 624 bp, Drosophila melanogaster
aaacaacactagcgcaagatggctgctctgctctcgtgtgaattttttgtttaaattaaaatcgcccgtggtgcggactgtttgagcaatcgaattttattggcacgccatgcgtgggatgaactaagacggatgcaagtgcggatatgcatcagcatcaacggaggagggccgaaaatacggagaatacaatcgaaaattccacaaaaccaacagccaggtgtcggtgtgtgtatgcgtgagagtgcttgtgcaagtgtatgtgtgtatttatattcttcatatcggatcggatcgtatcggaaaggtggtgcaaatgtttataaatatatattaaggaacaccttactatatatatgcgtgataaatccaatgaaatgtgcatgtggctgatggcttaaagcgccagcacaggcatcggagtgggcagcgacgtcggcagcgcaccagcgtcggattggaatgttaactaagagcaataacaacaacatcaacaaccactgaagcaacggcagcaccaccagctacatcaaaaacaaaccaaaacatacaataaaaacatatagaaacagcaatagctgcaaacatcattgaaaattggacgaggaggcacaagccatgaag

SEQ ID NO: 6 - DNA, 630 bp, Drosophila melanogaster
  misc_feature (88)..(88): n is a, c, g, or t
ctctcgtgtgaattttttgtttaaattaaaatcgcccgtggtgcggactgtttgagcaatcgaattttattggcacgccatgcggcgnatgaactaagacggatgcaagtgcggatatgcatcagcatcaacggaggagggccgaaaatacggagaatacaatcgaaaattccacaaaaccaacagccaggtgtcggtgtgtgtatgcgtgagagtgcttgtgcaagtgtatgtgtgtatttatattcttcatatcggatcggatcgtatcggaaaggtggtgcaaatgtttataaatatatattaaggaacaccttactatatatatgcgtgataaatccaatgaaatgtgcatgtggctgatggcttaaagcgccacgacaggcatcggagtgggcagcgacgtcggcagcgcaccagcgtcaggattggaatgttaactaagagcaataacaacaacatcaacaaccactgaagcaacggcagcaccaccagctacatcaaaaacaaaccaaaacatacaataaaaacaaatagagacagcaatagctgcaaacatcattgaaaattggacgaggaggcacaagccaggaagactagccacgagaagagccagaagcccaagccgaa

SEQ ID NO: 7 - DNA, 741 bp, Drosophila melanogaster
caaaaacggttaaacaacactagcgcaagatggctgctctgctctcgtgtgaattttttgtttaaattaaaatcgcccgtggtgcggactgtttgagcaatcgaattttattggcacgccatgcgtgggatgaactaagacggatgcaagtgcggatatgcatcagcatcaacggaggagggccgaaaatacggagaatacaatcgaaaattccacaaaaccaacagccaggtgtcggtgtgtgtatgcgtgagagtgcttgtgcaagtgtatgtgtgtatttatattcttcatatcggatcggatcgtatcggaaaggtggtgcaaatgtttataaatatatattaaggaacaccttactatatatatgcgtgataaatccaatgaaatgtgcatgtggctgatggcttaaagcgccagcacaggcatcggagtgggcagcgacgtcggcagcgcaccagcgtcggattggaatgttaactaagagcaataacaacaacatcaacaaccactgaagcaacggcagcaccaccagctacatcaaaaacaaaccaaaacatacaataaaaacaaatagaaacagcaatagctgcaaacatcattgaaaattggacgaggaggcacaagccaggaagactagccacgagaagagccagaagcccaagccgaagccggaggaaaaccataaggagaaggaagcgatctcgagaaaggcgtccggcgagggtgatctgatcacct

SEQ ID NO: 8 - DNA, 20 bp, Drosophila melanogaster
tgtcttcaccagcaagcatc

SEQ ID NO: 9 - DNA, 20 bp, Drosophila melanogaster
ctcccttgctgactcctcac

(19) United States
(12) Patent Application Publication
(10) Pub. No.: US 2006/0156421 A1
(43) Pub. Date: Jul. 13, 2006

(54) High throughput screening methods for anti-metastatic compounds

(76) Inventor: Ross L. Cagan, Clayton, MO (US)

Correspondence Address: SONNENSCHEIN NATH & ROSENTHAL LLP, P.O. BOX 061080, WACKER DRIVE STATION, SEARS TOWER, CHICAGO, IL, 60606-1080, US

(21) Appl. No.: 11/323,151

(22) Filed: Dec. 30, 2005

Related U.S. Application Data
(63) Continuation-in-part of application No. 11/154,035, filed on Jun. 16, 2005.
(60) Provisional application No. 60/580,769, filed on Jun. 18, 2004.
(60) Provisional application No. 60/580,897, filed on Jun. 18, 2004.

(51) Int. Cl. A01K 67/033

(52) U.S. Cl. 800/3; 800/13

(57) ABSTRACT

High throughput methods for screening of anti-cancer compounds using Drosophila are described. The methods involve modifying the expression of dCsk and observing the effect of putative anti-cancer candidate compounds on resulting expressed characteristics in the Drosophila. Related animal models and apparatus are also provided.

### CROSS-REFERENCE TO RELATED APPLICATIONS

This application is a continuation-in-part of U.S. patent application Ser. No. 11/154,035 filed Jun. 16, 2005, which claims priority to U.S. Ser. No. 60/580,769 filed Jun. 18, 2004 and U.S. Ser. No. 60/580,897 filed Jun. 18, 2004. Each of the above references is incorporated herein by reference in its entirety.

### STATEMENT REGARDING FEDERALLY SPONSORED RESEARCH OR DEVELOPMENT

Not Applicable.

### INCORPORATION-BY-REFERENCE OF MATERIAL SUBMITTED ON A COMPACT DISC

The Sequence Listing, which is a part of the present disclosure, includes a computer readable form and a written sequence listing comprising nucleotide and/or amino acid sequences of the present invention. The sequence listing information recorded in computer readable form is identical to the written sequence listing. The subject matter of the Sequence Listing is incorporated herein by reference in its entirety.

### FIELD

The present invention relates in general to the field of drug assaying techniques, and in particular to a novel high throughput screening assay for screening libraries of candidate compounds for treating human diseases and conditions including cancer and cancer-related conditions.

### INTRODUCTION

Recent scientific and technological advances have introduced new opportunities and challenges for drug discovery research. The increased availability of chemical libraries, including peptide and oligonucleotide libraries, and robotic systems enable virtually simultaneous synthesis and testing of hundreds or thousands of compounds. However, while screening of large numbers of candidate compounds is a critical early step in drug discovery and development, it can also be a bottleneck.

High throughput screening (HTS) assays and techniques of various types are typically used to screen chemical libraries consisting of large numbers of small molecules for their ability to suppress or enhance disease processes. Cell-free assays provide, for example, identification of putative drug targets implicated in a specific disease condition, such as a specific enzymatic reaction. Cell-based assays, for example, can provide insights into mechanisms underlying disease pathogenesis, and can also provide information on possible toxicity of candidate compounds. In either case, the goal of such screening is to identify the most likely candidates or “lead compounds” for use in further drug discovery and developments efforts, and not to identify a specific drug. The strength of a particular screening technique lies substantially in its ability to rapidly and efficiently screen large libraries of compounds while remaining cost effective.

Automated HTS assays and techniques and robotic systems for drug discovery have been described. The ability to perform a wide variety of biochemical and molecular biology tests using automated systems is widely known, including the ability to perform tests utilizing enzymatic activity, ELISA, receptor binding, macromolecular interactions, protein expression, and protein folding and assembly. Screens are typically carried out using multi-well microtiter plates. In drug discovery, a typical example of high throughput capacity is about one hundred to a few hundred plates per week depending on desired number of data points, the time required for all underlying biochemical reactions to occur, and the relative complexity of the scoring system used to determine whether a compound has an effect. A premium therefore exists on methods that simplify and speed detection of assay results.

A small molecular weight compound high throughput screening system using genetically modified Drosophila melanogaster has been described in U.S. Pub. No. 2002/0026648 A1. Compounds of interest are microinjected into the open hemolymph of genetically manipulated Drosophila that have been modified with mutations within a selected signaling pathway of interest. However, microinjection of compounds of interest into numerous Drosophila is technically difficult, and is particularly so in a high throughput context where the ability to automate is especially important. In addition, delivery of candidate compounds by microinjection occurs more slowly and can miss orally absorbable drugs.

Accordingly, there remains a need for methods and related articles of manufacture that improve the ability to screen through chemical libraries consisting of large numbers of candidate compounds to rapidly and easily identify the most likely candidate compounds for further drug discovery and research efforts, and particularly with respect to potential therapeutics for treating human cancer and cancer-related conditions.

### SUMMARY

The present invention is based in part on the discovery that screenably distinct characteristics associated with metastatic cellular behavior can be induced by modified expression of certain genes in wild-type Drosophila. In particular, loss of the Csk ortholog in Drosophila (dCsk) activates the protein Src and reproduces certain aspects of cancer metastasis which can be readily scored in Drosophila to form the basis of useful screening methods and models. In another related aspect, the inventors have made the surprising discovery that metastatic behavior of cells in which dCsk expression modified is also affected by the microenvironment of subject cells. More particularly, the inventors' discoveries provide the basis for methods for high throughput screening of candidate compounds for cancer, and particularly therapy for treating cancer metastasis. Candidate compounds that demonstrate the ability to modify expression of these characteristics according to the methods of the invention are thereby identified as suitable candidates for further testing as therapeutic alternatives for cancer treatment of animals including humans. The methods and related apparatus and kits are easily practiced, avoid the need for complex microinjection systems, identify orally absorbable drugs, and are readily adapted to automated high throughput systems.

Accordingly, in one embodiment there is provided a method for high throughput screening of compounds comprising: inducing a screenably distinct characteristic in wild-type Drosophila larvae by modifying expression of one or more Drosophila genes, wherein the screenably distinct characteristic is associated with human tumor metastasis, exposing the Drosophila larvae to a compound that putatively modifies the screenably distinct characteristic, and screening the Drosophila to determine whether the compound modifies the screenably distinct characteristic. In an exemplary embodiment, the screenably distinct characteristic is a cellular activity that is associated with human tumor cell metastasis, such as cell migration, reduced sensitivity to apoptosis, or abnormal tissue growth. Inducing a screenably distinct characteristic in wild-type Drosophila using targeted expression of one or more Drosophila genes may include modifying expression of one or more of the following: Csk, Src, cadherin, P120-catenin, Rho1, Cdc42, Rac1, Jnk, the metalloprotease MMP2, or DIAP1. In an exemplary embodiment, modifying expression of one or more Drosophila genes comprises reducing or eliminating dCsk gene expression. The method may further comprise spatially limiting the reduction or elimination of dCsk gene expression. In an exemplary embodiment, the reduction or elimination of dCsk gene expression is spatially limited to the Drosophila developing eye. The spatial limitation of reduction or elimination of dCsk expression may be further limited within the eye to a plurality of discrete patches of cells having boundary cells adjacent to wild-type cells. In another exemplary embodiment, the reduction or elimination of dCsk gene expression is spatially limited to the Drosophila developing wing. The spatial limitation of reduction or elimination of dCsk expression may be further limited within the wing to a plurality of discrete patches of cells having boundary cells adjacent to wild-type cells. Reducing or eliminating dCsk gene expression in a developing Drosophila comprises, for example, using an RNA interference (RNAi) construct to effect reduction or elimination of dCsk. The method may also further comprise screening the Drosophila to determine whether the compound has a toxic effect on the Drosophila.

In another aspect, there is provided an animal model of human tumor metastasis comprising a Drosophila having spatially limited modified expression of dCsk. In one embodiment, dCsk expression is reduced in the Drosophila developing eye. Reduced dCsk expression is, in an exemplary embodiment, further spatially limited to the Drosophila developing eye. In another embodiment, reduced dCsk expression is further spatially limited in the eye to a plurality of discrete clonal patches having boundary cells adjacent to wild-type cells. In another embodiment, dCsk expression is spatially limited to the Drosophila developing wing. In another embodiment, reduced dCsk expression is further spatially limited in the wing to a plurality of discrete clonal patches having boundary cells adjacent to wild-type cells.

In another aspect, there is provided a bioassay method for assaying the effects of a candidate anti-metastatic compound, the method including modifying at least one of cell migration, sensitivity to apoptosis and abnormal tissue growth in a Drosophila by modifying expression of dCsk; and exposing the Drosophila to a compound that putatively modifies at least one of cell migration, sensitivity to apoptosis and abnormal tissue growth. In one embodiment, modifying expression of dCsk comprises spatially limiting the modified expression of dCsk. In another embodiment, spatially limiting the modified expression of dCsk comprises reducing dCsk expression and spatially limiting reduced dCsk expression to the Drosophila developing eye. In another embodiment, spatially limiting the modified expression of dCsk comprises reducing dCsk expression and spatially limiting reduced dCsk expression to the Drosophila developing wing. In other embodiments, reduced dCsk expression within the eye or within the wing is further spatially limited within the eye or the wing to a plurality of discrete clonal patches having boundary cells adjacent to wild-type cells.

In another aspect, there is provided a bioassay method comprising: in a first Drosophila reducing dCsk expression and spatially limiting reduced dCsk expression to the Drosophila developing eye, in a second Drosophila reducing dCsk expression in the Drosophila developing eye and further spatially limiting reduced dCsk to a plurality of discrete clonal patches having boundary cells adjacent to wild-type cells, exposing the first Drosophila and the second Drosophila to a compound that putatively modifies at least one of cell migration, sensitivity to apoptosis and abnormal tissue growth, and comparing the effect of the compound on at least one of cell migration, sensitivity to apoptosis and abnormal tissue growth in the first Drosophila and the second Drosophila.

In another aspect, there is provided apparatus for use in a high throughput screening assay method comprising a multi-well microtiter plate, an amount of a Drosophila growth medium placed into the multiple wells of the multi-well microtiter plate, an amount of a candidate compound added to the multiple wells, and at least one Drosophila in each of said multiple wells, the Drosophila having modified dCsk expression and modified expression of at least one screenably distinct characteristic. In one embodiment the modified expression of dCsk is reduced or eliminated. In one embodiment, at least one screenably distinct characteristic comprises a characteristic associated with human tumor metastasis. The characteristic associated with human tumor metastasis is, for example, one of cell migration, sensitivity to apoptosis and abnormal tissue growth. In one embodiment, the screenably distinct characteristic comprises abnormal tissue growth in the wing. In another embodiment the screenably distinct characteristic comprises abnormal tissue growth in the eye.

These and other features, aspects and advantages of the present teachings will become better understood with reference to the following description, examples and appended claims.

### DRAWINGS

Those of skill in the art will understand that the drawings, described below, are for illustrative purposes only. The drawings are not intended to limit the scope of the present teachings in any way.

FIG. 1 is an exemplary multiwell microtiter plate apparatus for high throughput screening of compounds using Drosophila.

FIG. 2 is a sampling of photomicrographs of Drosophila omatidia showing wild-type omatidia and overgrowth phenotype resulting from targeting MEN2A-analogous and MEN2B-analogous forms of dRet.

FIG. 3 shows electron photomicrographs of omatidia illustrating modification of a screenably distinct phenotype by a candidate compound, in which the candidate compound strongly inhibits in dose-dependent fashion the severity of the rough eye phenotype of both dRet and dRet^{MEN2B}.

FIG. 4 shows dCsk-dependent retina phenotypes.

FIG. 5 shows in situ visualization of developing dCsk retinas.

FIG. 6 shows wing phenotypes for dCsk cells.

FIG. 7 shows position and profile of boundary dCsk cells.

FIG. 8 shows anti-cleaved Caspase-7 staining (red) from wing discs with various genotypes.

FIG. 9 shows the effect of p120ctn on the removal of dCsk boundary cells.

FIG. 10 shows a model for the behavior of dCsk cells.

FIG. 11 shows that broad expression of the dCsk-IR transgene phenocopies dCsk mutants.

FIG. 12 shows SEMs of adult eyes with co-expression of dE-cadherin/Shotgun.

FIG. 13 shows mutant clonal tissue in a rarely obtained dCsk-IR FLP/FRT-mediated clone (hs-FLP; tub>GFP>gal4; UAS-dCsk-IR).

FIG. 14 shows (A) Western blotting from control (GMR-gal4/+) and GMR>dCsk-IR retinas at 29 hr APF with anti-DIAP1 and anti-Actin antibodies, and (B and B′) show anti-DIAP1 antibody staining (red) from a wing disc with the genotype ptc>dCsk-IR; ptc>GFP.

FIG. 15 shows anti-cleaved Caspase-7 staining from wing discs with the genotypes sd>dCsk-IR (A) and sd>dCsk-IR; sd>puc (B).

FIG. 16 shows anti-cleaved Caspase-7 staining (red) from wing discs with the genotypes ptc>dCsk-IR; ptc>GFP (A), ptc>dCsk-IR; ptc>GFP; ptc>hChk (B), and enhanced-GFP expressed with two copies of patched (ptc>GFP; ptc>GFP) (C).

FIG. 17 shows specificity for Rho1 GTPase activity at dCsk boundary cells.

### DETAILED DESCRIPTION

Abbreviations and Definitions

To facilitate understanding of the invention, a number of terms and abbreviations as used herein are defined below as follows:

“Altered form”: As used herein with respect to a gene, the term “altered form” refers to a gene which differs from a given gene sequence by one or more mutations such as a single point mutation, such that the activity of the gene is modified but not eliminated.

“Drosophila”: As used herein, “Drosophila” refers to an insect or insects belonging to the fruit fly species Drosophila melanogaster, without regard to developmental stage thereof and including embryos (eggs), larvae, pupae and mature adult flies of the species.

“Eye”: As used herein, “eye” broadly refers to the visual organ of Drosophila, including retinae at any stage of development.

“Mimic”: as used herein, the term “mimic” refers to the action of resembling or imitating a human disease or condition by producing characteristic symptoms of the disease, for example in the way that abnormal tissue growth is said to mimic cancer.

“Wild-type”: As used herein, “wild-type” refers to Drosophila having a genome that has not been genetically modified or manipulated in a laboratory, for example by recombinant techniques.

“To screen”: As used herein, “to screen” refers to the act of examining a group of organisms, such as Drosophila, and using the expression of a selected characteristic as a criterion for separating the organisms into at least two groups.

“Screenably distinct”: As used herein, the term “screenably distinct” refers to a characteristic of a Drosophila individual or individuals, or to the Drosophila individual per se, that deviates from the wild-type individual Drosophila in such a way that visual inspection or other simple detection methods can be used to detect the presence of the characteristic, wherein the presence or absence of the characteristic is used as the criterion for screening the organisms into at least two groups. A screenably distinct characteristic may be a feature of a genotypic variant of wild-type Drosophila in the sense that the characteristic may result from a Drosophila gene or transcript that is orthologous to a human oncogene or tumor suppressor and is stably placed within the Drosophila and expressed in the Drosophila.

“Variant”: As used herein, the term “variant” refers to a Drosophila individual that deviates from wild-type individual Drosophila with respect to at least one characteristic.

“Oncogene”: As used herein, “oncogene” refers to a gene or transcript that is capable, when it has higher than normal activity, of inducing abnormal tissue growth due to effects on the biology of a cell, for example on the cell cycle or cell death process.

“Tumor suppressor”: As used herein, “tumor suppressor” refers to a gene or transcript that is capable, when it has lower than normal activity, of inducing abnormal tissue growth due to effects on the biology of a cell, for example on the cell cycle or cell death process.

“Activity”: As used herein, “activity” refers to the level of functioning in which a gene or transcript participates; for example, high activity of a gene or gene product refers to an increase in the gene's function relative to its normal level of functioning.

“Targeted expression”: As used herein, “targeted expression” refers to the manipulation of a gene or transcript through the use of a transgene to induce its expression in one or more tissues within the Drosophila.

“Transgene”: As used herein, “transgene” refers to an artificially constructed stretch of DNA that, for example, can be placed into a Drosophila by stable integration in the Drosophila's genome.

“Embryo(s)”: As used herein, “embryo” and “embryos” refer to the egg stage of Drosophila melanogaster.

“Toxic”: As used herein, “toxic” and “toxicity” refer to a characteristic of a compound that through its chemical action kills, injures or impairs an organism.

“dCsk”: As used herein, “dCsk” refers to the gene or transcript having a sequence of Flybase Accession No. CG17309 (GenBank Accession No. AE003692; SEQ ID NO: 1) available at http://flybase.bio.indiana.edu/.bin/fbidq.html?FBgn037925, or the protein encoded by said locus.

“Csk”: As used herein, “Csk” refers to a gene or transcript or protein that is an ortholog of dCsk and is found in organisms other than Drosophila.

“dRet”: As used herein, “dRet” refers to the gene or transcript having a sequence of Flybase Accession No. CG1061 (GenBank Accession No. NM_{—}057697; SEQ ID NO: 2) available at http://flybase.bio.indiana.edu/.bin/fbidq.html?FBgn0011829&content=full-report, or the protein encoded by said locus.

“Ret”: As used herein, “Ret” refers to a gene or transcript or protein that is an ortholog of dRet and is found in organisms other than Drosophila.

“To plate”: As used herein, “to plate” refers to the act of placing material, including growth medium, candidate compounds, and Drosophila embryos, into wells of a microtiter plate.

“Phenotype”: As used herein, “phenotype” refers to the outward manifestation of the action of a gene due to the gene's gain or reduction in activity, for example the aberrant development of the Drosophila eye due to reduction of dCsk activity.

High Throughput Screening Methods for Anti-Metastatic Compounds

The present invention provides methods and related are based in part on the discovery that targeted expression of oncogenes or tumor suppressors, or orthologs thereof, produces screenably distinct characteristics in Drosophila that then serve as a basis for discriminations within the context of a high throughput screening system. The present invention takes advantage of the novel combined use of a Drosophila line having a transgene-induced screenable characteristic, and a technique for high-throughput compound screening.

More specifically, expression of a transgene in Drosophila is modified, such that the functionality of dRet in Drosophila is increased, or alternatively, the dCsk functionality in Drosophila is reduced. The transgene expression is modified, for example, by engineering a single point mutation into a transgene, and establishing a stable transgenic line of individuals having the transgene. The transgene expression can also be modified using an RNAi construct, such as siRNA's as known in the art to produce targeted inhibition of gene expression. In either case, the modified gene expression that alters dRet or dCsk functionality in Drosophila, leads to the formation of an abnormal retina in the Drosophila. The abnormal retina is a screenably distinct characteristic in the Drosophila, in that it is a characteristic of a Drosophila individual or individuals that deviates from wild-type individual Drosophila so clearly that visual inspection or other simple detection methods can be used to detect the presence or absence of the abnormal retina. The presence or absence, and comparative level of abnormality when present, is then assessed and compared between Drosophila to which a candidate therapeutic compound has been administered, and Drosophila to which no compound or a control compound has been administered, and the comparison used to determine whether the candidate compound has any effect on the screenably distinct characteristic.

Accordingly, methods, related apparatus and kits for high throughput screening assays involve the preparation of microtiter plates each with multiple wells, wherein each well initially contains one or more Drosophila embryos with a transgene and an amount of a Drosophila growth medium. The embryos develop while feeding on the growth medium. The precise age of the embryos at the time they are plated matters less than the fact that they are all about the same age, to permit accurate evaluation of the possible effects of the candidate compound on larval and pupal development.

To prepare the microtiter plates, for example, 96-well microtiter plates are used, such as those commonly commercially available and typically used for various laboratory assay techniques, including other high throughput drug assay techniques. Into each well is pipetted 50-100 μl of standard Drosophila growth medium. An exemplary range of about 50 to about 100 μl is a balance between (i) providing sufficient food so as not to place undue feeding stress on the developing flies and (ii) providing sufficient air space for the third larval instars to find sufficient wall space to pupate and for minimal stress on the developing larvae and pupae. Any one of several standard Drosophila growth medium recipes as known in the art of breeding Drosophila for research can be used.

In an exemplary embodiment, a candidate compound, or cocktail of more than one compound, that has been selected for screening is prepared in EtOH or DMSO/aqueous solution. In an exemplary embodiment, EtOH is used. Although DMSO can be used, it can be toxic if it reaches final concentrations of more than 0.3% of the growth medium. The compound in solution is added and allowed to diffuse through the growth medium for an initial period of about 16 to about 24 hours. Alternatively, the compound in solution is mixed with the food by pipetting, by shaking, or by sonicating. Drosophila embryos of the desired genotype or containing the desired transgene are collected en masse and, after the initial period of diffusion of the candidate compound through the growth medium, sorted several to a well. In an exemplary embodiment, five to six embryos are sorted to each well. However, the number of embryos in each well can easily vary, provided that no more embryos than will flourish in the well are used. The number of embryos per well will also be influenced by the need to obtain a sufficient number of data points to make the test meaningful.

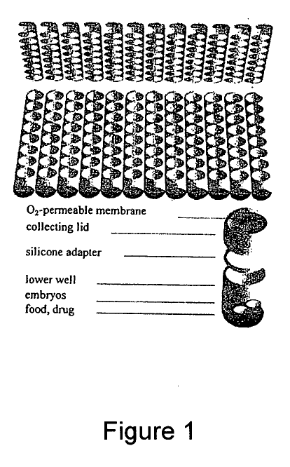

Once the Drosophila embryos are placed into each well on the growth medium, they hatch out and begin feeding after a second period of about 24 hours, bringing the final amount of diffusion time for the subject compound to about 40 to about 48 hours. A period of about 24 to about 48 hours is sufficient for full diffusion of most compounds. In some cases where adequate diffusion of the compound does not occur within a period of about 48 hours, the growth medium in the plate can be warmed and then sonicated to facilitate mixing of the candidate compound with the growth medium. Finally, each well is sealed by placing a second microwell plate in inverted orientation so that the opening at the top of each well is closely apposed; this second microwell will ideally have a membrane or covering at the base of each well that will permit (i) flow of sufficient oxygen to allow the developing Drosophila to thrive and (ii) the containment of the Drosophila within each compartment formed by the apposition of the two plates. An exemplary such covering is the Millipore Multiscreen-FC MAFCNOB10. In an exemplary embodiment, the two plates are further aligned and sealed by an intervening adaptor to yield the configuration as shown in FIG. 1. It is anticipated that other configurations and components can be utilized that will yield the same or suitably similar results.

Assaying Methods

In one embodiment of the methods, a method for high throughput screening of compounds includes inducing a screenably distinct characteristic in Drosophila by modifying expression of an oncogene or a tumor suppressor in the Drosophila, feeding to embryos of such altered Drosophila a compound that putatively modifies the screenably distinct characteristic, and screening the Drosophila to determine whether the compound modifies the screenably distinct characteristic. In one exemplary embodiment, reducing the activity of dCsk in the developing Drosophila retina with an introduced transgene results in a screenably distinct retina. In another exemplary embodiment, expressing an activated form of dRet in the Drosophila's retina with an introduced transgene results in a screenably distinct retina. It is anticipated that other approaches that alter the development of the eye can be utilized that yield a similar result.

The Drosophila retinae can be screened as described in the Examples, infra. For example, the screenably distinct characteristic of a Drosophila retina with a reduction in dCsk can be examined after (i) growing Drosophila with said distinct characteristic in microwells containing standard Drosophila media plus a compound that putatively modifies the distinct characteristic, (ii) permitting said Drosophila to advance in their development in said microwells, and (iii) screening the ability of said compound to alter the perceived severity of the retina's distinct characteristic.

The severity of a Drosophila retina's distinct characteristic can be easily determined by a screening step involving examining the retina surface through a standard dissecting microscope plus a suitable light source. In one exemplary example, the severity of a Drosophila retina's distinct characteristic can be assessed by determining overall size of the retina, the total number of omatidia, the proper alignment of the constituent omatidia, whether two neighboring omatidia are abnormally close together or fused, and whether the retina folds abnormally within its normal niche on the head.

Lethality of candidate compounds for Drosophila can be used to detect and quantify toxicity of candidate compounds. Well known standard statistical methods are used to help distinguish chance results from real toxic effects. Lethality is quantified, for example, by determining the number of Drosophila that fail to develop successfully to adulthood and applying suitable statistical analyses to determine statistical significance. Lethal dose evaluations can be used to quantify the extent of toxicity. For example, once a candidate compound demonstrates a mediating effect on a reduction of dCsk activity or an increase in dRet activity, the toxicity of the compound is evaluated by varying dosage levels across a broad range and quantifying the lethality of the compound at each dose to obtain an LD₅₀ value. In an exemplary example, (i) a consistent and significant reduction in the number of adults within a microwell or (ii) the presence of dead or dying Drosophila within a microwell is taken as evidence that a compound is significantly toxic. In another exemplary example, the emergence of most Drosophila adults within a microwell indicates a lowered probability that an introduced compound is toxic.

In one embodiment there is provided a method for high throughput screening of compounds comprising: inducing a screenably distinct characteristic in wild-type Drosophila larvae by modifying expression of one or more Drosophila genes, wherein the screenably distinct characteristic is associated with human tumor metastasis. As described in the present disclosure, the screenably distinct characteristic is, for example, one of cell migration, sensitivity to apoptosis and abnormal tissue growth, each of which characterize metastatic behavior of cells. The Drosophila larvae are then exposed to a candidate compound that putatively modifies the screenably distinct characteristic. The Drosophila are then monitored to determine whether the compound modifies the screenably distinct characteristic.

Targeted expression of one or more Drosophila genes according to the present methods includes modifying expression of one or more of the following: Csk, Src, cadherin, P120-catenin, Rho1, Jnk, the metalloprotease MMP2, or DIAP1. In an exemplary embodiment of the methods, dCsk gene expression is reduced or eliminated. The modification of gene expression can be accomplished using any known techniques for modifying expression of a particular gene, including but not limited to use of antisense sequences or RNA interference (RNAi). The method may further comprise spatially limiting the reduction or elimination of dCsk gene expression. For example, the reduction or elimination of dCsk gene expression is spatially limited to the Drosophila developing eye, and does not affect any remaining structures. The spatial limitation of reduction or elimination of dCsk expression may be further limited within the eye to a plurality of discrete clonal patches having boundary cells adjacent to wild-type cells. Similarly, the reduction or elimination of dCsk gene expression can be spatially limited to the Drosophila developing wing, and further limited within the wing to a plurality of discrete clonal patches having boundary cells adjacent to wild-type cells.

The present disclosure also embraces a novel animal model of human tumor metastasis comprising a Drosophila characterized by modified expression of dCsk which is spatially limited. For example, in one embodiment of the animal model, dCsk expression is reduced, and the reduction in activity is limited to the Drosophila developing eye. Reduced dCsk expression is further spatially limited in the eye to a plurality of discrete clonal patches having boundary cells adjacent to wild-type cells. Similarly, in the animal model, the reduction or elimination of dCsk gene expression in the Drosophila can be spatially limited to the developing wing, and further limited within the wing to a plurality of discrete clonal patches having boundary cells adjacent to wild-type cells.

In another aspect, there is also provided a bioassay method for assaying the effects of a candidate anti-metastatic compound. At least one of the human tumor metastasis-associated characteristics including cell migration, sensitivity to apoptosis and abnormal tissue growth is modified in a Drosophila by modifying expression of dCsk. The Drosophila is then exposed to a compound that putatively modifies at least one of the metastasis-associated characteristics, and effect of the compound on same is then observed. In one embodiment of the bioassay method, modifying expression of dCsk comprises spatially limiting the modified expression of dCsk, which includes for example, reducing dCsk expression and spatially limiting reduced dCsk expression to the Drosophila developing eye. Alternatively, spatially limiting the modified expression of dCsk comprises reducing dCsk expression and spatially limiting reduced dCsk expression to the Drosophila developing wing. As described herein, the metastatic-associated characteristic can be further modified by further spatially limiting the reduction of dCsk activity within the eye or within the wing to a plurality of discrete clonal patches having boundary cells adjacent to wild-type cells.

In another aspect, a bioassay method is provided in which metastatic behavior of cells is manipulated and compared depending on the extent of spatial limits on the controlled expression. Drosophila that vary in terms of the spatial limitation of expression can then be used to compare the relative effects of candidate anti-metastatic compounds. More specifically, the method includes reducing dCsk expression in a first Drosophila and spatially limiting the reduced dCsk expression to the Drosophila developing eye, reducing dCsk expression in a second Drosophila developing eye and further spatially limiting reduced dCsk within the eye to a plurality of discrete clonal patches having boundary cells adjacent to wild-type cells. The first Drosophila and the second Drosophila are then exposed to a candidate compound that putatively modifies a metastatic cell behavior such as cell migration, sensitivity to apoptosis or abnormal tissue growth. The effect of the compound on at least one of the metastatic cell behaviors is then observed and evaluated.

Apparatus for Use in HTS Methods

In another aspect, the invention provides apparatus for use in high throughput screening methods as described herein. The apparatus includes a multi-well microtiter plate, an amount of a standard Drosophila growth medium placed into multiple wells of the multi-well microtiter plate, an amount of a candidate compound added to the multiple wells, and a plurality of screenably distinct Drosophila in the multiple wells, the screenably distinct Drosophila having developed from Drosophila embryos altered in a manner useful for studying a specific oncogene or tumor suppressor, or for studying metastatic behavior of cells. The screenably distinct Drosophila include, for example, Drosophila with a reduced or eliminated level of dCsk activity, or Drosophila with an increased level of dRet activity. According to the present methods, dCsk activity is reduced or eliminated in spatially limited manner, for example by limiting the reduced or eliminated dCsk activity to the eye or to the wing. In the case of reduced or eliminated dCsk activity, the screenably distinct characteristic is, for example, cell migration, and sensitivity to apoptosis or abnormal tissue growth. A Drosophila with at least one screenably distinct characteristic is then placed into a multi-well microtiter plate with a suitable lid to (i) permit Drosophila survival and development and (ii) prevent escaping of developing Drosophila.

Also provided according to the present disclosure is apparatus for use in a high throughput screening assay method for screening potential anti-metastatic compounds. The apparatus includes, for example, a multi-well microtiter plate with multiple wells, an amount of a Drosophila growth medium placed into the multiple wells, an amount of a candidate compound added to the multiple wells, and at least one Drosophila in each of said multiple wells, wherein each Drosophila has modified dCsk expression and modified phenotypic expression of at least one screenably distinct characteristic. In one embodiment the modified expression of dCsk is reduced or eliminated expression of dCsk gene, which produces at least one screenably distinct characteristic associated with human tumor metastasis. Such characteristics include, for example, cell migration, sensitivity to apoptosis and abnormal tissue growth. Of particular utility as a screenably distinct characteristic is abnormal tissue growth in the wing or abnormal tissue growth in the eye, both of which can be readily visually observed, documented and measured using well-known and readily available techniques in tissue staining, microscopy and imaging as described herein, as well as in other references known in the art.

Automated Screening

Preparation of the microtiter plates with the growth medium, Drosophila embryos and candidate compounds can be performed manually or using a robotic system or systems. For example, plating of the growth medium and of candidate compounds in solution on the microtiter plates can be readily adapted to known robotic systems that can be configured to repeatedly inject a predetermined volume of the growth medium and of the test solutions into each well of the microtiter plate. Similarly, the assay results can be determined manually, or can be adapted to automated or robotic analyzers.

Kits

Further, the present invention provides a kit for use in a method for high throughput screening of compounds. The kit includes instructions for the following: instructions for inducing a screenably distinct characteristic in Drosophila containing a mutation or transgene that creates a screenably distinct characteristic, instructions for feeding to the Drosophila embryos a compound that putatively modifies the screenably distinct characteristic, and instructions for screening the Drosophila to determine whether the compound modifies the screenably distinct characteristic. In one embodiment, the instructions set forth more specifically instructions for screening the Drosophila to determine whether the compound modifies alterations in the screenably distinct phenotype in the Drosophila. In still another embodiment, the instructions set forth instructions for determining whether the compound has a toxic effect on the Drosophila. In yet another embodiment, the kit further includes a multi-well microtiter plate, and an amount of a Drosophila growth medium for placement into multiple wells of the multi-well microtiter plate. The kit can still further include the lid for sealing each well of the multi-well microtiter plate.

Relationship to Multiple Endocrine Neoplasias

Multiple Endocrine Neoplasias (MENs) are dominant, inherited, familial cancer syndromes. They are characterized by a variety of tumors of the endocrine glands arising from neuroendocrine cells. Multiple Endocrine Neoplasia II (MEN II, or MEN2) is a hereditary disorder in which patients develop a type of thyroid cancer accompanied by recurring cancer of the adrenal glands. One type of this disease (MEN IIa, or MEN2a) is also associated with overgrowth (hyperplasia) of the parathyroid gland. MEN2 syndromes are defined by medullary thyroid carcinoma (MTC), a potentially aggressive tumor prone to widespread metastases that is generally refractory to radiation and chemotherapy. The cause of MEN2 is a mutation in a gene called Ret. The disorder affects all ages and both genders equally. A family history of MEN2 is the primary risk factor.

The Ret gene encodes a tyrosine kinase receptor for neurotrophic molecules. Gene rearrangements, including specific point mutations, activate the oncogenic potential of Ret in human thyroid papillary carcinomas. Different point mutations activate Ret in familial multiple endocrine neoplasia syndromes. Inactivating mutations of Ret are present in some Hirschsprung's disease patients. Increasingly detailed knowledge of the specific Ret mutations responsible for human tumors provides important tools for the clinical management of these diseases.

“C-ret” is a proto-oncogene (normal gene having the potential for change into an oncogene) of Ret, which encodes a 120 kD transmembrane receptor with a tendency to re-arrange during transfection (Takahashi et al., 1985). C-Ret is expressed in a variety of tissues, primarily derivatives of the neural crest such as components of the autonomic and enteric nervous system and regions of the Wolffian duct and ureteric bud epithelium (Takahashi et al, 1998; Tsuzuki et al., 1995). Deletion of Ret activity in mice leads to renal dysgenesis and loss of enteric neurons (Schuchardt et al., 1994). This and a variety of related work has indicated that Ret plays a central role in the proliferation, differentiation, and migration of cells during renal organogenesis and enteric neurogenesis and likely a variety of other organs as well. In addition, the c-ret locus represents a ‘hotspot’ for oncogenic mutations.

Ligand-mediated activation of Ret leads to dimerization, auto-phosphorylation, and activation of the receptor. MEN2A mutations achieve ligand-independent activation by promoting dimerization; MEN2B mutations can bypass requirement for dimerization. The intracellular domain of Ret contains a tyrosine kinase catalytic domain that is necessary for its activity. Ligand-mediated activation of Ret leads to tyrosine phosphorylation and subsequent binding of a phospholipase C, and the Shc, SNT/FRS2, IRS1, Dok, and GRB2 adapters: in addition to ras, activated Ret can stimulate jnk, PI-3K/AKT, src, and p38 signaling (Alberti et al., 1998; Arighi et al., 1997; Besset et al., 2000; Borrello et al., 1994; Califano et al., 2000; Hayashi et al., 2000; Kurokawa et al., 2001; Melillo et al., 2001a; Melillo et al., 2001b; Ohiwa et al., 1997; Pelicci et al., 2002; Soler et al., 1999), and Enigma can bind and promote signaling in a phosphorylation-independent manner (Durick et al., 1998). The short form of Ret can bind both the PTB and SH2 domains of Shc, whereas the long form binds exclusively the PTB domain; the functional significance of this difference is not well understood. Interestingly, the hRet^{MEN2B} mutant forms, described below, bind exclusively to the PTB domain; again, the functional significance of its inability to bind the SH2 domain is also unclear (Ohiwa et al., 1997).

Five human syndromes are associated with mutations within the c-ret locus; in addition, somatic c-ret mutations are associated with sporadic medullary thyroid cancer. Hirschsprung's disease represents point mutations or breakpoints that reduce receptor activity, leading to intestinal aganglionosis and renal dysplasia. Activating point mutations can be classified into four groups: FMTC, Ret/PTC, MEN2A, and MEN2B. Familial Medullary Thyroid Carcinoma (FMTC) is characterized by one of several point mutations that lead to medullary thyroid carcinomas (MTCs). Mutations associated with FMTC appear to be weakly activating; most alter extracellular cysteines that provoke spontaneous activation, though some mutations target residues within the tyrosine kinase domain (Donis-Keller et al., 1993; Eng et al., 1996; Mulligan et al., 1994; Pasini et al., 1997; Pasini et al., 1996). Papillary thyroid carcinomas are commonly linked to rearrangements that create a chimeric receptor and spurious activation of a number of downstream targets (reviewed in Tallini, 2002).

Nearly all MEN2A patients contain a mutation that alters one of five cysteines (C609, C611, C618, C620, or C634) within the extracellular domain. The result is ligand-independent dimerization and strong activation of the receptor (Donis-Keller et al., 1993; Mulligan et al., 1994; Mulligan et al., 1993). This leads to a series of oncogenic events, particularly MTCs, pheochromocytomas (adrenal medulla tumors), and parathyroid adenomas.

A more severe form of MEN2 is typically the result of a methionine-to-threonine substitution at position 918 (M918T) within the tyrosine kinase catalytic domain of hRet (Carlson et al., 1994a; Hofstra et al., 1994); rarely (<5%), other residues in Ret are targeted (Menko et al., 2002). The result is MEN2B, a debilitating disease also characterized by medullary thyroid carcinomas and pheochromocytomas; in addition, ganglioneuromas, mucosal neuromas, megacolon, a generalized neural hypertrophy, early defects in bone structure including marfinoid habitus, and possibly other developmental defects are commonly observed (reviewed in Takahashi, 1997). MEN2B mutations have also been associated with aganglionosis leading to congenital megacolon, more commonly associated with Hirschsprung's disease (Romeo et al., 1998). In both MEN2A and MEN2B, studies indicate the importance of prophylactic thyroidectomies: multifocal MTC and C cell hyperplasia were consistently found in youth as young as 6 years (Lallier et al., 1998).

Although MTC is a relatively uncommon form of thyroid cancer, the morbidity and mortality rates are significant. At present, there are no effective non-surgical therapies for the treatment of medullary thyroid carcinoma. Pre-symptomatic or prophylactic thyroidectomy in hRet disease allele carriers may be curative. However, most patients with MEN2B have metastatic disease involving nearby lymph nodes (levels II-V) at the time of diagnosis. Although there have been significant advances in the detection and surgical excision of metastatic disease in the neck region, surgery rarely provides a cure (Lips et al., 1994; Moley et al., 1998; Wells, 1994).

Early detection is central to the successful management of medullary thyroid carcinoma. Unfortunately, pre-symptomatic diagnosis and early surgical intervention is rarely possible for most MEN2B patients with MTC. There are two impediments to early identification and treatment. The first relates to the frequency with which new MEN2B mutations appear in the population. More than half of all patients with MEN2B have de novo disease (Carlson et al., 1994b); the lack of similarly affected family members leads to diagnosis at an age that is typically later than in multi-generational MEN2B kindreds. The second obstacle to early detection is a lack of specific symptoms in patients with MTC, an issue especially common to sporadic disease. Sporadic MTC usually presents as a palpable neck mass at a later age and at a higher stage than inherited forms of MTC (Wells, 1994).

Currently, surgery remains the only effective therapy for MTC; metastatic MTC is not responsive to radiation or chemotherapy. These surgeries are complex and tedious, and repeat procedures are common. A better understanding of the abnormal signaling that occurs in tumors with the hRet^{MEN2B} mutant receptor would help us identify better therapeutic targets. New agents to control and cure MTC are needed for the successful management of this group of patients. Inherited forms of MEN2B are not especially common; however, it is of note that the M918 mutation is likely the most frequent hRet defect seen in sporadic (somatic) MTC. As is the case with MEN2B patients, surgery is rarely curative.

The human M918T allele is the most malignant of the hRet mutations described to date. M918T accounts for more than 95% of MEN2B patients characterized, and 30%-80% of sporadic MTCs (Eng et al., 1996; Eng et al., 1994). Although mutations other than threonine at position 918 can lead to weak activation of the receptor, only threonine appears able to transform Ret into an oncogenic form (Cirafici et al., 1997). In addition, unlike hRet^{MEN2A} mutations, M918T-mediated receptor activation does not lead to or require dimerization of the receptor. When an analogous mutation was made in the Ron (M1254T) and Met (M1250T) receptor tyrosine kinases, the result was activation of the Ras signaling pathway and—similar to Ret^{MEN2B}—apparent activation of another signal transduction pathway (Bardelli et al., 1998; Santoro et al., 1998); this is likely due to alteration of the ‘activation loop’, which regulates access to the kinase domain (Miller et al., 2001).

The cysteine mutations seen in Ret^{MEN2A} are likely to open the structure to spontaneous disulfide bonding and dimerization. Second-site mutagenesis studies indicated that Ret^{MEN2A} receptors require tyrosine 905 for signaling whereas Ret^{MEN2B} receptors require tyrosines 864 and 952, suggesting the potential for differences in the signaling targets of these two receptors (Takahashi et al., 1998). Ret^{MEN2B} receptors also fail to phosphorylate the tyrosine at position 1096, normally required for binding of the Grb2 adapter protein (Liu et al., 1996). Finally, Ret^{MEN2A} and Ret^{MEN2B} demonstrate different responses to GDNF ligand. In the presence of GDNFRδa, Ret^{MEN2B} proved responsive to GDNF and phosphorylated the downstream target Shc, whereas Ret^{MEN2A} was poorly responsive (Bongarzone et al., 1998; Carlomagno et al., 1998). The precise pathway(s) activated by the M918T mutation in Ret^{MEN2B} is unknown. Several possible pathways have been suggested, including Src-like and JNK signaling, Nck, Crk, and Paxillin (Bocciardi et al., 1997; Marshall et al., 1997; Songyang et al., 1995); however, the evidence for activation of any of these pathways in vivo has been lacking, and in vivo work failed to detect differences in intracellular signaling between the two MEN2 isoforms (see below).

Engineering MEN2A-analogous and MEN2B-analogous oncogenic forms of Ret into mice has yielded mixed results. Targeted expression of Ret^{MEN2A} isoforms directs MTC formation in mice, although the penetrance is variable; they also developed C-cell and follicle tumors (Acton et al., 2000; Michiels et al., 1997; Reynolds et al., 2001). Attempts to create an MEN2B model mouse has also been partially successful: introducing the M918T mutation into endogenous Ret led to C-cell hyperplasia, pheochromocytomas, and occasional ganglioneuromas, although the penetrance for many of the defects was low and other abnormalities seen in humans such as developmental defects were absent (Smith-Hicks et al., 2000). The normal development observed in homozygous M918T mice indicated that the MEN2B form of Ret still signals normally in addition to its transforming potential.

Src Family Cytoplasmic Tyrosine Kinases (SFKs) and Disease

Normal development requires strict spatial and temporal control of cellular processes such as proliferation and differentiation in order for properly sized and functioning organisms to form. This control is achieved through a network of signal transduction pathways that coordinate developmental events between cells, tissues, and organs. Inappropriate activation of these signal transduction networks can cause diseases such as oncogenesis in which individual cells respond to aberrant internal cues to overproliferate and overgrow. Src family cytoplasmic tyrosine kinases (SFKs) play important roles within these networks to regulate both developmental events and disease states. Humans and mice have at least eight SFKs, including Src, Fyn, and Yes. Many of these kinases have been linked to developmental events such as morphogenesis and to diseases such as oncogenesis, but the exact roles of SFKs in these processes remain ambiguous.

SFKs are composed of a tyrosine kinase domain, an SH2 domain, an SH3 domain, and a regulatory C-terminal region. They can be activated by receptor tyrosine kinases (RTKs), cytokine and immune receptors, G-protein coupled receptors, and integrins. SFK activation can cause cell cycle entry, cytoskeletal rearrangements, and alterations in cell adhesion, while disruption of SFK function can inhibit cell migration. Mammalian tissue culture models have identified numerous downstream effectors of SFK functions; these include signaling molecules in the Ras/ERK, Jun kinase, Jak/STAT, PI-3 kinase, and Rac/Rho pathways. However, SFK activities have not been well explored in vivo, in part due to functional redundancy among SFKs. For example, src−/− mice show only subtle osteoclast defects, while src−/−, fyn−/−, yes−/− mouse embryos show early lethality and multiple developmental anomalies including neural tube defects and dramatically reduced size. Fibroblasts derived from src−/−, fyn−/−, yes−/− mice show reduced proliferation, suggesting that some of the phenotypes of compound knock-out embryos are caused by proliferative defects during development. However, the precise role of Src, Fyn, and Yes in cell cycle during development remains unknown.

SFKs are maintained in an inactive state through tyrosine phosphorylation of their C-terminal region by the negative regulator C-terminal Src kinase (Csk), which itself is closely related to SFKs. Deletion or mutation of the Csk target site leads to upregulation of SFK kinase activity. Mammals have two Csk family members, Csk and Chk. Mice deficient for Csk show hyperactivation of SFKs and a striking embryonic phenotype also characterized by early lethality, neural tube defects, and reduced size. Surprisingly, csk−/− fibroblasts do not show increased proliferation, which conflicts with data indicating that increased SFK activity leads to cell cycle entry. This may reflect functional compensation by Chk, which also negatively regulates SFKs. This redundancy between multiple SFKs and Csk kinases as well as the early lethality of Csk and compound SFK knockouts has impeded detailed evaluation of SFK function in developing mammalian tissues.

Abnormal constitutive activation of SFKs has been implicated in oncogenesis, but its precise role is also ambiguous. Numerous human tumors possess activated SFKs, but SFK mutations have been found in only a fraction of these tumors. Some human colon cancers harbor mutations that abolish the ability of the C-terminal domain to inhibit Src kinase activity. The transforming v-Src oncogene shows deletion of the Csk target site. Since SFKs can be abnormally activated through disregulation of the C-terminal region, reduced Csk family kinase activity could promote oncogenesis. Yet, the role of Csk and/or Chk in tumors is controversial or unclear. Large deletions within the region of chromosome 15 that harbors Csk have been observed in colon cancers, the tumor types that commonly show elevated SFK activity, but no specific loss-of-function Csk mutations have been found in tumors to date. Reduced Csk expression and function is correlated with Src activation in primary hepatocellular tumors, primary colorectal tumors, and colon carcinoma cell lines. However, others have reported elevated Csk in tumors with high SFK activity. In addition, Csk^{-/-} primary mouse fibroblasts do not show a transformed phenotype. Perhaps mutations in other loci, such as Chk, are required to reveal a tumor suppressor function for Csk. A detailed exploration of Csk's function in vivo is required to better understand its role in disease and development, but, again, such studies have been impeded by the early lethality of Csk^{-/-} mice.

The imaginal discs of Drosophila provide a powerful model system for the study of signal transduction. Imaginal discs share several properties with mammalian epithelial tissues: both are composed of epithelial cells that must maintain proportional growth, differentiation, and renewal in order to form functional tissues and organs. Cells within imaginal discs undergo proliferation and differentiation in response to molecular pathways that have been highly conserved across species and that function in oncogenesis. For example, studies of the eye imaginal disc have provided important evidence that the Ras and Jak/STAT signal transduction pathways are crucial for normal growth, proliferation, and differentiation. Recent genetic analyses of ‘tumor suppressor’ mutations have led to new insights about known human tumor suppressors and identification of new putative human tumor suppressors such as lats and salvador.

The Drosophila genome contains two SFKs, Src42A and Src64B, that are functionally similar to their mammalian counterparts. Src42A and Src64B loss-of-function mutations disrupt cytoskeletal regulation within developing oocytes and embryos. Yet, the full repertoire of SFK functions remains to be elucidated in Drosophila. Src42A and Src64B are regulated by a Csk-like activity in flies, but until now the gene responsible for that activity was unknown. The cloning and characterization of the Drosophila Csk ortholog dCsk are described herein. Loss of dCsk function led primarily to overgrowth phenotypes in developing tissues such as the eye; genetic data indicated that excess proliferation was due to upregulation of SFKs. Evidence is provided that this overgrowth requires the JNK and STAT signal transduction pathways. Reducing STAT function prevented growth and normal differentiation of dCsk mutant tissue, instead provoking dCsk^{-/-} cells to undergo apoptosis. The data provide in vivo evidence for a Src-dependent pro-apoptotic pathway triggered by reduced STAT function. They are consistent with results from Stewart et al (Stewart, 2003). Together, these results connect SFK signaling to the cell cycle and suggest an approach for restraining its proliferative potential.

Materials and Methods

Flies were grown at 25° C. Fly stocks were obtained from the Bloomington Stock Center unless otherwise noted. S030003 and S017909 were from the Szeged Stock Center. Src64B^{P1} was a gift of M. Simon. Stat92E^{j6C8} was a gift of S. Hou. Src42A^{Su1} and Src42A¹⁸⁻² were gifts of X. Lu. To create EGUF clones, y w: ey-Gal4 UAS-FLP/+; FRT82B GMR-hid I(3)CL-R/FRT82B dCsk flies were established by standard crosses; w; FRT82B GMR-hid I(3)CL-R/FRT82B Ubi-GFPnIsS65T flies were utilized as controls for minor artifacts inherent in the EGUF system. dCsk^{j1D8/S030003} trans-heterozygotes showed an intermediate phenotype and were used to examine genetic interactions between dCsk and candidate genes.

Genomic and EST analysis: sequence flanking the j1D8 and S030003 P-element insertions was generated and mapped by the Berkeley Drosophila Genome Project (BDGP) and Szeged Stock Center, respectively. The following CG17309 (SEQ ID NO: 1) ESTs were obtained from BDGP and fully sequenced: LD36541 (GenBank Accession No. AI456502; SEQ ID NO: 3), LP09923 (GenBank Accession No. BT011103; SEQ ID NO: 4), GH10267 (GenBank Accession No. Al 113804; SEQ ID NO: 5), LD22810 (GenBank Accession No. M940695; SEQ ID NO: 6), and LD33364 (GenBank Accession No. AA979099; SEQ ID NO: 7). Sequences were assembled, compared, and analyzed with BLAST, MultAlin, PROSCAN, and Genestream.

Rescue and Reversion: to create the heat-shock inducible dCsk transgene hs-dCsk, the LD22810 cDNA was cloned into pPCaSpeR-hs, and stable insertions were created. dCsk^{j1D8}, dCsk^{S030003}, and dCsk^{S017909} were extensively out-crossed to remove observed background mutations. w; hs-dCsk/+; dCsk/dCsk and w; +/+; dCsk/dCsk embryos were collected for 3-4 days in vials. Larvae were heat shocked at 37° C. for 30 minutes every 10-16 hour to induce dCsk expression. For reversion, S017909 and S030003 were excised by standard crosses; over 10 independent excisions were scored for reversion of lethality j1D8 failed to excise.

Larval and pupal body size measurements: embryos were collected for 4 hours and larvae were grown at similar densities. For mass measurements, larvae were cleaned and weighed in groups of 15-20 on a Mettler AE50 balance. A minimum of 3 groups was measured for each genotype at each time point. Average body mass was calculated by determining the average of the sum of the average body mass per group. Values for each time point were normalized to the average mass of wild-type control larvae. For pupal measurements, pupae were photographed and relative length measurements were taken from printed enlargements. Values were normalized to wild-type pupae.

Clonal analysis and flow cytometry: flow cytometry was performed generally as described (Neufeld, 1998 #4340). Dissociated imaginal discs cells were run on a Cytomation MoFlo Cytometer. Data was analyzed in Summit v3.1 (Cytomation). For analysis of loss-of-function clones, the genotypes were: y w hs-FLP/+: FRT82B Ubi-GFPnIsS65T/FRT82B dCsk^{j1D8} and y w hs-FLP/+; FRT82B Ubi-GFPnIsS65T/FRT82B dCsk^{S030003}. Clones were induced by heat shock at 48 and 72 hours AED and dissected at 120 hours. GFP positive and negative tissues were used to control for GFP detection. FACS experiments were repeated at least 3 times. Direct scoring of clonal patches within the eye disc was not relied on, in part because the boundaries of the clones could not be reliably distinguished using available reagents.

Histology, Immunohistochemistry, and SEM: In situ hybridization was performed as described (Tautz, 1989) using a probe to the 5′ end of both dCsk transcripts bounded by an Nco1 and Bsg1 site. Negative controls lacked probe. Digoxigenin was detected with an alkaline phosphatase conjugated antibody (Behringer Manheim).

For adult sections, heads were fixed in 1% glutaraldehyde/2% osmium tetroxide/PBS, dehydrated and washed, and incubated 4 hours in 1:1 propylene oxide Durcupan ACM resin, overnight in 100% resin, and finally at 65° C. to harden. Serial sections were stained with 0.5% methylene blue/0.1% toluidine blue. Digital photographs were taken on a Zeiss Axioplan.

For immunohistochemistry, tissue was fixed for 20 minutes in 4% paraformaldehyde with 1×PBS or 1×PEM and stains were performed in 1×PBS, 10% FBS, 0.3% Triton-X100. Antibodies to affinity purified anti-Stat92E was used at 1:500 (Chen, 2002 #4507), anti-phospho-histone H3 (Upstate Biotechnology) at 1:200, and 22C10 and active-capase-7 (New England Biolabs) at 1:4 and 1:50, respectively. Secondary antibodies were conjugated to Alexa Red or Green (Molecular Probes). For dCsk mitotic clones, ey-FLP/+; FRT82B Ubi-GFPnIsS65T/FRT82 dCsk were used. Digital photographs were taken on a Zeiss Axioplan.

To estimate mitotic activity printed enlargements of phospho-histone stains of EGUF discs were examined. To control for tissue mass, phospho-histone positive nuclei within a quadrant of fixed size were counted and the number of positive nuclei within identically sized fields of tissue for each genotype were recorded. Nuclei were counted in 3 quadrants per disc and the average number of mitotic nuclei per quadrant was determined.

For SEM, adult flies were fixed in 95% ethanol, re-hydrated, treated with 1% osmium tetroxide, dried, and sputter coated. Ommatidia were counted on printed enlargements of SEM micrographs. For dCsk^{j1D8} EGUF clones, estimates of ommatidia were made using SEMs of the entire eye plus separate SEMs to visualize folds.

dCsk Encodes a Negative Regulator of Growth and Proliferation

In a screen for mutations that genetically modify an over-expressed, oncogenic form of the Ret receptor tyrosine kinase in Drosophila three P transposable elements that enhanced the activated Ret phenotype were identified. Fly lines j1D8, S030003, and S017909 contain P-element insertions within the CG17309 locus. Five (5) of 50 known CG17309 ESTs were fully sequenced and it determined that CG17309 encodes two nearly identical predicted proteins that differ only at the N-terminus. The predicted proteins contain a tyrosine kinase domain and an SH2 domain that, together, show the highest homology with Csk family kinases. In fact, CG17309 proteins show a higher homology to Csk orthologs from other species such as mouse, Xenopus, and Hydra than to any other Drosophila tyrosine kinase. They also contain a glutamine-rich region in place of the SH3 domain found in mammalian Csk proteins. Consistent with other members of the Csk family, CG17309 proteins lack an N-terminal myristoylation signal and lack a C-terminal negative regulatory tyrosine present in SFKs. Also, CG17309 proteins lack plextrin homology and Tec-homology domains, which distinguish them from the closely related Tec-Btk family tyrosine kinases. Previous analyses of the Drosophila genome have concluded that CG17309 encodes the sole Drosophila Csk ortholog. Based on these data and data presented below, this locus is referred to as Drosophila Csk ortholog, or dCsk, and the three insertion lines as dCsk^{j1D8}, dCsk^{S030003}, and dCsk^{S017909}.

All three dCsk lines are lethal and displayed a stronger phenotype when in trans to a deficiency. dCsk^{j1D8} exhibited the earliest lethal phase, dying within 6-18 hours after pupation, a lethal phase which overlapped with that of dCsk^{j1D8} in trans to deficiency, illustrating that dCsk^{j1D8} is a strong hypomorphic mutation. Excision of the dCsk^{S0300003} and dCsk^{S017909} insertions reverted their lethality and/or non-complementation with dCsk^{j1D8}. In situ hybridization indicated that dCsk mRNA is ubiquitously expressed within developing larval tissues. dCsk^{j1D8}, dCsk^{S030003}, and dCsk^{S017909} mutant tissues showed reduced dCsk expression by in situ hybridization. Finally, heat shock-induced expression of a dCsk cDNA rescued the lethality and mutant phenotypes in all three dCsk alleles. By itself, ectopic, ubiquitous expression had no detectable effect on the adult phenotype. These data demonstrate that all three P element insertions disrupt the dCsk locus.

During fly development, embryos hatch to progress through three larval stages followed by pupation and metamorphosis. dCsk mutants occasionally survived through later pupal development, allowing for characterization of dCsk larvae and pupae. The most striking phenotype of dCsk mutants was their increased body size relative to wild-type animals. Early third instar dCsk larvae weighed 30% more than age-matched wild-type larvae and eventually grew to weigh 84% more than wild-type larvae due to a prolonged larval stage in which they continued to feed and grow long after wild-type controls had pupated. dCsk pupae displayed a 21% increase in body length vs. controls. Wandering dCsk mutant larvae showed enlargement of tissues such as the brain, ventral ganglion, and salivary glands, and enlargement of the wing, leg, and eye imaginal discs.

Pharate adults are animals that attain a near adult morphology but die within the pupal case. The eyes and heads of the occasional dCsk^{j1D8/S030003} and dCsk^{S030003} mutants that survived as pharate adults were frequently enlarged and posterior ommatidia were sometimes misaligned. Histological sections indicated that individual mutant ommatidia were morphologically normal (data not shown) but contained more ommatidia than wild-type controls. Rarely, the eyes were replaced with duplicated antennae. In addition, the wings and legs were severely malformed, the notum was sometimes ‘split’, and the head, legs, and notum often contained cuticle outgrowths.

To resolve the origin of the retinal defects, the EGUF system was utilized to generate ‘whole eye clones’ in which all adult eye tissue is homozygous for dCsk mutations in an otherwise heterozygous animal. This approach permitted us to isolate dCsk activity within the retina from, e.g., effects of the prolonged larval stage; flies with eyes homozygous for dCsk developed along a normal time course. dCsk EGUF clones were also enlarged in comparison to controls, with some dCsk^{j1D8} clones so enlarged that the eyes became malformed in order to pack onto a normally sized head. Occasionally, dCsk EGUF clones resulted in antennal duplication and cuticle overgrowth, phenotypes that recapitulated defects seen in dCsk pharate adults.

The enlarged dCsk EGUF eyes contained an increased number of ommatidia. The cells within these retinas were normal in morphology and size, though some ommatidia exhibited planar polarity inversions. Retinal cell proliferation occurs almost exclusively within the embryonic and larval eyes, and the observed extra cells most likely derive from excess proliferation during these stages. Importantly, previous studies show that blocking apoptosis does not affect eye size. Consistent with over-proliferation, late larval eye-antennal imaginal discs from dCsk EGUF clones were enlarged compared to age-matched controls and showed an increase in proliferating cells. These data indicate that dCsk acts to regulate organ size and cell proliferation within the developing eye field.

To further explore dCsk's cell proliferation defects and to determine whether it acts autonomously within individual cells, fluorescence-assisted cell sorting (FACS) analysis was utilized in whole eyes and in Flp-FRT-generated clones. First, FACS analysis demonstrated that dissociated cells from whole dCsk mutant eye-antennal and wing discs consistently exhibited a decrease in the G0-G1 population and an increase in the G2-M population when compared to cells from age-matched control tissues; these results are consistent with a similar analysis in the wing. These differences were observed in cell cycle profiles in mutant larvae over a range of ages, from 120 hr to 130 hr AED. Similar results were observed in dCsk EGUF larval eyes. To assess whether the defects observed in dCsk mutants are cell autonomous, the Flp-FRT system was used to generate mutant clones within the eye; to rigorously score the effects on individual cells, the cells were dissociated and FACS analysis used to segregate the dCsk homozygous clonal cells from their wild-type and heterozygous neighbors. Again, dCsk mutant clones contained an increased G2-M population and a decreased G0-G1 population relative to surrounding control tissue, a cell cycle defect indicative of increased proliferation. Non-dCsk cells were unaffected. Forward scatter measurements confirmed that dCsk homozygous clonal cells and their neighbors were the same average cell size even in different phases of the cell cycle. Together, these data argue that dCsk controls tissue growth cell autonomously by negatively regulating cellular proliferation without affecting cell size, although subtle non-autonomous effects cannot be ruled out.

dCsk Acts in Opposition to the Src and JNK Pathways

A dCsk^{j1D8/S030003} trans-heterozygote combination was used to test candidate loci for an in vivo role in dCsk function. Several candidate genes such as members of the Ras pathway failed to genetically interact with dCsk. The dCsk phenotype was suppressed by mutations in the Drosophila Src ortholog Src64B. Normally, 10-40% of developing dCsk flies survived to pharate stages and only 0-1% eclosed (emerged) from their pupal cases. Removing one copy of Src64B led to fully 61% surviving at least as pharate adults, and 26% of these eclosed from their pupal cases. The eclosed adults often displayed wing and leg defects, and typically died within 24-48 hours. Mutations in the Src ortholog Src42A weakly suppressed dCsk phenotypes: 56% of dCsk mutants either eclosed or lived to the pharate stages when one copy of Src42A was removed using the Src42A¹⁸⁻² allele.

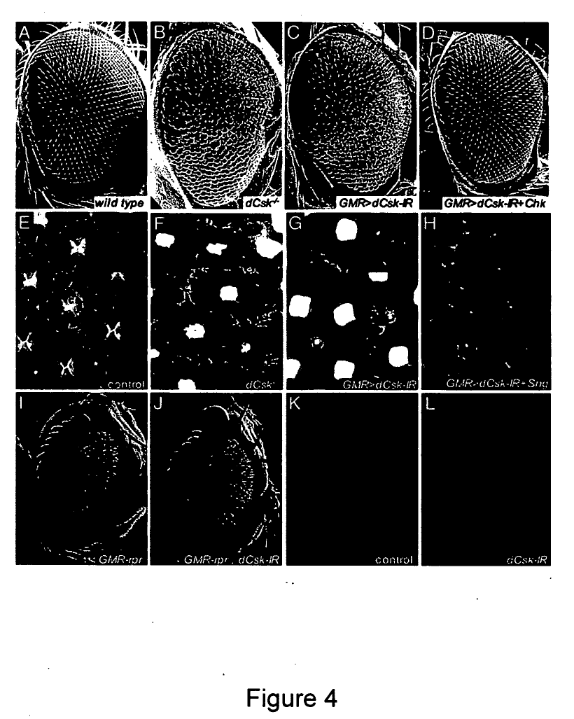

The Btk29A locus encodes the sole Tec-Btk family kinases in the Drosophila genome, which function downstream of fly Src kinases such as Src64B. Mutations in Btk29A strongly suppressed dCsk: 70% of Btk29A/+; dCsk flies fully eclosed as nearly normal adults (FIGS. 4A, 4D) and exhibited only mild wing defects. In addition, reduced Btk29A function also noticeably suppressed the increased body size and prolonged larval phase observed in dCsk mutants (data not shown). FACS analysis of dissociated wing and eye-antennal imaginal discs derived from Btk29A/+; dCsk larvae indicated that removal of a copy of Btk29A suppressed the increase in G2-M cells observed in dCsk mutants, demonstrating that Btk29A mediates the cell cycle defects observed in dCsk mutants.

The Jun N-terminal kinase (JNK) signaling pathway has also been identified as a mediator of Src signaling in both mammals and Drosophila. Consistent with this data, removing one copy of the JNK ortholog basket (bsk) also suppressed the dCsk phenotype. 60% of bsk¹/+; dCsk^{j1D8/S030003} flies formed viable adults that fully or partially eclosed. Similar to Src64B; dCsk survivors, these adults exhibited leg and wing defects and died shortly after eclosion. Larvae and pupae also showed suppression of the increased body size (data not shown). FACS analysis indicated that larval eye-antennal discs contained an increased G0-G1 and decreased G2-M population relative to control discs, demonstrating that mutations in bsk suppress the cell cycle defects caused by loss of dCsk.

dCsk Negatively Regulates Jak/Stat Signaling

Another pathway linked to Src signaling in mammalian tissue culture models is the Jak/Stat signal transduction pathway: Src can directly phosphorylate and activate STAT3 in vitro, and STAT3 function and activation are required for Src transforming activity in multiple tissue culture cell lines. In the Drosophila eye, the Jak/Stat pathway controls proliferation and planar polarity. The Drosophila Jak/Stat pathway is composed of the ligand Unpaired (Upd), the receptor Domeless, the single Jak ortholog Hopscotch (Hop), and the single STAT ortholog Stat92E. Recent work has demonstrated that over-expression of Upd leads to STAT pathway-dependent overproliferation and ommatidial polarity defects in the eye very similar to those seen in the dCsk EGUF clones. Removing one copy of Stat92E suppressed the Upd overexpression phenotype, indicating that the Upd phenotype was sensitive to alterations in Jak/Stat function. Conversely, removing one copy of dCsk enhanced eye overgrowth caused by Upd over-expression, demonstrating that dCsk negatively regulates the Jak/Stat pathway in this paradigm.

One indicator of Drosophila Jak/Stat activity is Stat92E protein levels: upd and hop mutant flies show decreased Stat92E protein expression and Upd over-expression in the eye leads to increased Stat92E protein. Cells fully mutant for dCsk exhibited a clear elevation in Stat92E protein levels relative to wild-type or heterozygous eye tissue. This increase indicates that the Jak/Stat pathway is up-regulated in dCsk mutants and suggests that this up-regulation may provoke some of the cellular defects observed in dCsk eyes.

The dCsk Phenotype Requires Stat92E Function

To further explore the role of Stat92E in dCsk function, the EGUF system was utilized to create eyes fully mutant for both dCsk and Stat92E. Eyes mutant for Stat92E alone were mostly normal, showing a slight reduction in size, some misaligned ommatidia and, infrequently, missing antennal structures. Genotypically dCsk^{j1D8}; Stat92E⁰⁶³⁴⁶ EGUF eyes—the two loci are linked on the same chromosomal arm—were consistently and often significantly smaller than either dCsk^{j1D8} or Stat92E⁰⁶³⁴⁶ EGUF eyes alone, demonstrating a block in the overgrowth phenotype. In addition, dCsk; Stat92E adult eyes were frequently fragmented, with scars and/or patches of eye tissue separated by patches of cuticle, suggesting that mutant tissue underwent localized programmed cell death during development. Doubly mutant flies also exhibited a loss of antennal structures and head cuticle malformations. The cuticle malformations were present on animals with small and scarred eyes suggesting that these malformations are secondary to retinal defects. All of these observations were confirmed in dCsk^{j1D8}; Stat92E^{j6C8} flies, which demonstrated an even higher penetrance of eye tissue loss.

To determine if the observed defects in dCsk; Stat92E EGUF clones were Src-dependent, one copy of Btk29A was removed in dCsk^{j1D8}; Stat92E⁰⁶³⁴⁶ EGUF clones. If the reduced eye size of dCsk; Stat92E EGUF clones was due to Src hyperactivation, then reduced Btk29A function should ‘rescue’ the dCsk; Stat92E phenotype; if, however, the phenotype was the result of nonspecific synthetic lethality then it should not be sensitive to reduction of Btk29A function. Consistent with former possibility, reduced Btk29A suppressed and rescued the dCsk; Stat92E eye to a more normal phenotype. In particular, while 64% of all adult dCsk; Stat92E eyes were two-thirds or less of normal size, only 21% of all adult eyes from Btk29Ak^{k00206}/+; dCsk; Stat92E eyes were that small. Also, 77% of the Btk29A/+; dCsk; Stat92E eyes were normal or nearly normal in size, whereas only 32% of dCsk, Stat92E EGUF eyes were similarly normal. Indeed, most Btk29A/+; dCsk; Stat92E EGUF clones looked very similar to Stat92E EGUF clones, as both genotypes showed some misaligned ommatidia and, occasionally, missing antennal structures.

To determine the developmental origin of the dCsk; Stat92E EGUF phenotype, eye-antennal imaginal discs were examined. dCsk^{j1D8}; Stat92E⁰⁶³⁴⁶ mutant larval eye-antennal discs frequently showed significantly reduced size relative to control, Stat92E, or dCsk EGUF clones, a reduction often also observed in developing antennal tissues. dCsk; Stat92E EGUF eyes showed reduced mitoses anterior to the morphogenetic furrow compared to control or dCsk^{j1D8} clones. In addition, doubly mutant eye tissue often exhibited patchy expression of neural markers and decreased proliferation relative to control or dCsk^{j1D8} tissue. Regions with reduced neural development harbored cells with abnormal and pyknotic nuclei as visualized with DAPI staining (data not shown), suggesting that cells within the eye were undergoing apoptosis. Consistent with this data, dCsk^{j1D8}; Stat92E⁰⁶³⁴⁶ mutant larval eye tissue often exhibited increased programmed cell death and tissue loss within the developing eye field. This apoptosis primarily occurred in regions with reduced neural marker expression, indicating that defective neural differentiation may occur as a consequence of excessive apoptosis during development. Such extensive apoptosis is likely to account for much of the tissue loss and scarring observed in adult dCsk; Stat92E EGUF clones. In summary, reduced Stat92E activity inhibited SFK-mediated overgrowth in dCsk mutant tissue by reducing cell proliferation and promoting apoptotic cell death.

The Effect of Local Cellular Microenvironment on dCsk Deficient Cells

Loss of dCsk in discrete patches led to a different outcome: epithelial exclusion, invasive migration, and apoptotic death. These latter phenotypes required sharp differences in dCsk activity between neighbors; dE-Cadherin, P120-catenin, Rho1, Jnk, and MMP2 mediated this signal. The outcome of a cell's loss of dCsk is linked to its cellular microenvironment. When dCsk activity was reduced broadly in the developing eye or wing, the result was over-proliferation, inhibition of apoptosis, and decreased cell adhesion. Tissue integrity was retained, but dCsk cells were inappropriately mobile and failed to maintain their appropriate contacts. The outcome of these effects was an overgrown and miss-patterned adult tissue. By contrast, loss of dCsk in discrete patches resulted in epithelial exclusion, invasive migration through the basal extracellular matrix, and eventual apoptotic death; these events occurred exclusively at the boundary between dCsk and wild-type cells. Further emphasizing the unique nature of cells at this boundary, a specific requirement was observed for a signal that includes Drosophila orthologs of E-cadherin, P120-catenin, RhoA, Jnk, and the metalloprotease MMP2.

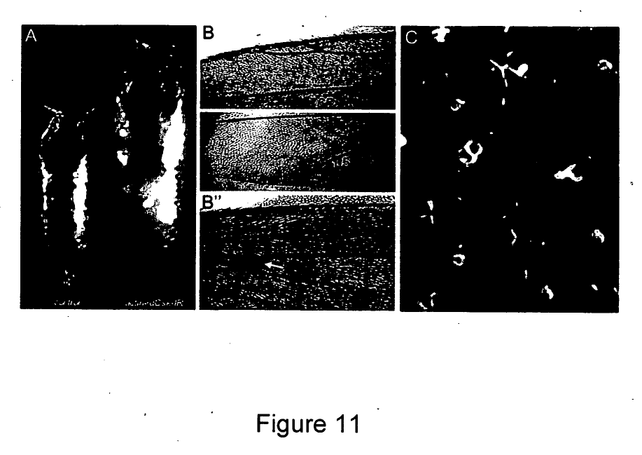

To further explore dCsk functions beyond cell proliferation, the dCsk transcript was targeted for reduction by RNA interference (RNAi) through the use of an inverted repeat (IR)-containing transgene. Use of the GAL4/UAS system (Brand and Perrimon, 1993) achieved a high degree of temporal and spatial control of expression. For example, GMR-GAL4; UAS-dCsk-IR (‘GMR>dCsk-IR’) flies were generated with expected reduced dCsk activity specifically in the developing eye. FIG. 4 shows dCsk-dependent retina phenotypes. (A-D) show scanning electron micrographs (SEMs) from (A) wild-type, (B) dCskc04256 (C) GMR>dCsk-IR and (D) GMR>dCsk-IR; GMR>hChk adult eyes. (E-H) show the results of anti-Armadillo staining of retinas at 42 hr APF from GMRgal4/+(E), dCskc04256 EGUF (F), GMR>dCsk-IR (G), and GMR>dCsk-IR; GMR>dEcadherin shotgun (H). (I) displays GMR-reaper eyes, which are small and rough due to ectopic cell death. (J) shows that the GMR-reaper phenotype was partially suppressed in the presence of GMR>dCsk-IR. (K-L) show TUNEL staining of wild-type or GMR>dCsk-IR retinas at 29 hr APF. The phenotype of GMR>dCsk-IR (i) showed an enlarged and rough eye phenotype (FIG. 4C), (ii) enhanced the effects of missexpressing the Src isoform dSrc64B but not those of dSrc42ACA, a constitutively activated isoform of dSrc42A that lacks the consensus regulatory tyrosine targeted by Csk (data not shown) and (iii) was suppressed by co-expression of the human ortholog CHK (FIG. 4D). The latter result also indicated that human Chk could at least partially replace dCsk function, providing additional evidence for conservation of Csk/Src signaling across metazoa (Miller et al., 2000). Ubiquitous expression of dCsk-IR (actin5C>dCsk-IR) phenocopied dCsk mutants in other tissues as well: for example, animals died as young pupae, with pupal body size approximately 50% larger than their wild-type controls (FIG. 11). Each of these phenotypes is similar in type and severity to those observed in flies containing the strong hypomorphic alleles dCskJ1D8 (Read et al., 2004) and dCskc04256 (data not shown).

To better understand how inactivation of dCsk can alter tissue growth and patterning, the developing Drosophila eye was examined. Retinal cell fates first emerge in the mature larval eye as staggered sets of eight photoreceptor neurons and four (glial-like) cone cells coalesce into discrete ommatidia. By the end of larval development, cell division has essentially ceased in the eye (apart from bristle cell precursors). In the pupa, two primary pigment cells (1°s) are added to complete each fourteen-cell ommatidial cluster. The remaining interommatidial precursor cells (IPCs) that lie between ommatidia undergo selective programmed cell death (PCD) and cell rearrangements that assemble them into a precise, interweaving hexagonal lattice of secondary and tertiary pigment cells (2°/3°) and sensory bristles. This lattice organizes the ommatidial array (FIG. 4E).

Phenocopying dCskc04256 (FIG. 4F), the numbers and arrangement of 2°s and 3°s were defective in GMR>dCsk-IR eyes (FIG. 4G). A marked increase in cell number was observed, and the normally hexagonal pattern of the lattice was disrupted. The extent of the patterning disruptions varied regionally across the ommatidial field as cells piled up around some ommatidia, were nearly absent around others, and were found at approximately correct numbers in other regions. Even areas that contained approximately correct numbers of IPCs cells were miss-patterned, indicating that loss of dCsk activity affected both the number of cells and also cell patterning or morphogenesis. Reduction of dCsk activity affected development by mechanisms that are independent of cell cycle control, as all cell divisions are complete well before the stage of IPC patterning.

At 29 hrs after puparium formation (APF), GMR>dCsk-IR pupal retinas showed a striking reduction in programmed cell death as assessed by TUNEL (FIGS. 4K and L). Furthermore, GMR>dCsk-IR suppressed the apoptosis-related phenotypes that resulted from miss-expression of the pro-apoptotic genes Reaper and Hid (FIG. 41 and J; data not shown); removing a single functional copy of dCsk (dCskj1D8/+) had similar effects (data not shown). Taken together, these data indicate that, independent of its activity in regulating cell division, a broad loss of dCsk can result in a block in apoptosis. Reducing dCsk activity also led to abnormal 2°/3° patterning (FIG. 4F-G).

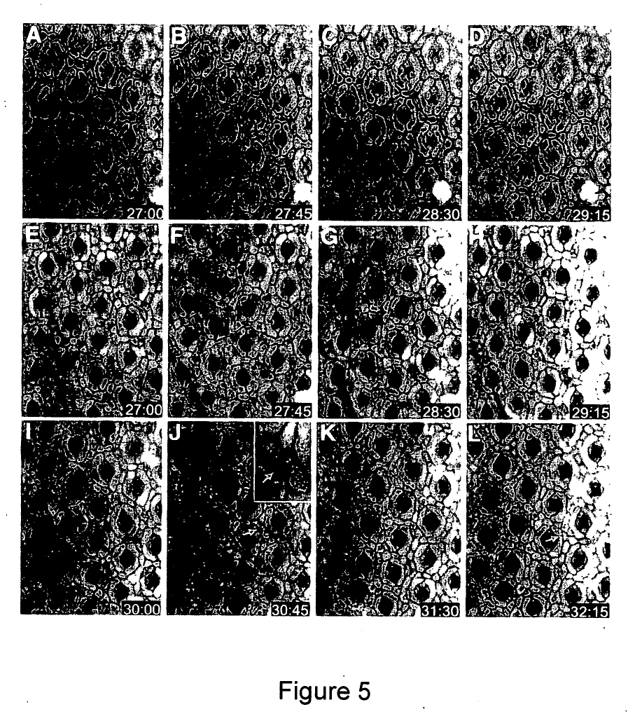

Recent data indicated that IPCs assemble into a hexagonal pattern based on their preferential adhesion to 1°s: IPCs initially become more mobile, they re-arrange between 1°s into a hexagonal pattern, which is stabilized by re-establishment of stable cell-cell junctions (Bao and Cagan, 2005; Grzeschik and Knust, 2005). Therefore, dCsk-mediated disruption of 2°/3° patterning could be due to either a failure of IPCs to move into their proper cell niches or a failure to maintain those positions. To resolve these issues, a technique was developed to visualize retina morphogenesis in situ with single cell resolution. The pupal cuticle over the eye was removed, αCatenin-GFP was used to visualize individual cells at the surface, and high-resolution snapshots were taken every 10-20 minutes. In wild-type pupal retinas, the majority of IPC morphogenetic movements took place between 18-27 hr APF (C. Brachmann, D. Larson, and R. C., unpublished results). By 27 hr APF, IPCs moved from multiple cell layers to single file around 1°s (FIG. 5A). At this stage, cells have begun to push between each other to establish first (i) stable 3°s at the vertices and then (ii) the 2° array. Concurrently, excess cells were removed by PCD (FIG. 5A-D).

In contrast, the movies from GMR>dCsk-IR retinas virtually never detected removal of cells from the interommatidial lattice even at the stages when PCD is normally maximal, confirming that loss of PCD is a primary cause of ectopic interommatidial cells in dCsk eyes (FIG. 5E-L). Although most IPCs moved initially into their proper niches, a striking inability of these cells to hold their positions was observed. For example, a cell that contacts three 1°s normally settles stably as a 3°; Supplementary movie 2 shows examples of cells that move into and then out of the 3° niche. Cells were observed to move several cell lengths away, gliding over other cells to form abnormal ‘piles’ of cells (e.g., green cells in FIG. 5E-L). Often, these too robust, aberrant cell movements disorganized already-patterned areas (e.g., orange cells in FIG. 5E-L).

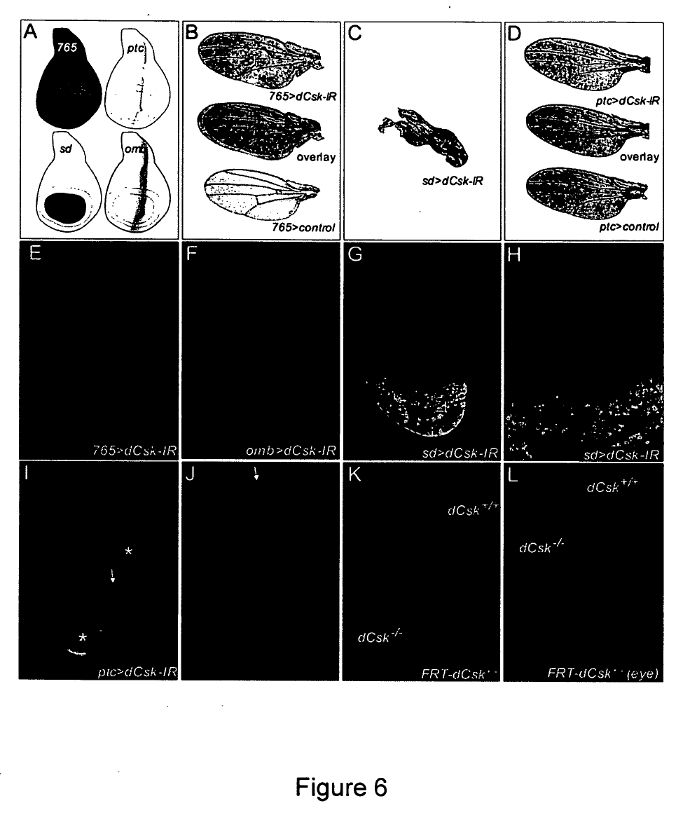

To determine whether the effects observed with dCsk-IR were exclusive to the eye epithelium, the transgene to the wing disc was targeted using 765-GAL4, which directs expression throughout the developing wing disc (Gomez-Skarmeta et al., 1996). 765>dCsk-IR adult flies contained significantly larger wings than control animals (7.17% larger on average; p<0.05; FIG. 6B), consistent with over-proliferation and a lack of compensatory apoptosis. Interestingly, patterning defects were also observed: extra vein material was present in the L2 and L5 veins, and the posterior cross vein contained extra branches (FIG. 11B).

Discrete Inactivation of dCsk Leads to Cell Migration and Death

Other loci have been described that, when mutated, give rise to enlarged eye and wing discs; these include bantam, salvador, archipelago, hippo, and lats/warts. Clonal patches mutant for any of these loci overgrow relative to their neighbors (Harvey et al., 2003) due to their ability to direct both overgrowth and a block in cell death. Reducing dCsk activity throughout the embryo, eye, or wing also led to an increase in animal and organ size. Therefore, it was a surprise to find that discrete clonal patches of dCskj1D8, dCskc04256, or dCskSO300003 mutant cells rarely survived to pupal or adult stages. The rare pupal clones that were obtained were significantly smaller than their simultaneously created ‘twin spot’ controls (e.g., FIGS. 6K-L and 13B).

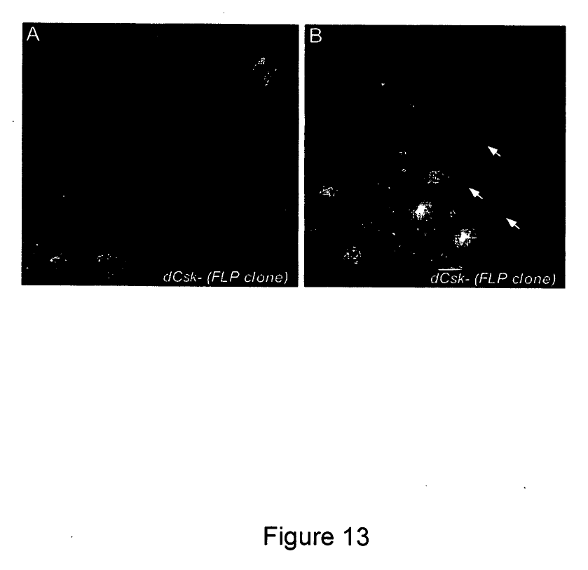

To explore this issue further, an FLP-out approach was employed (Basler and Struhl, 1994) to express dCsk-IR in discrete clonal patches. Again, attempts to create early, large FLP-out clones in the young embryo failed to produce detectable pupal clones and were frequently lethal to the organism (data not shown). One rare example is shown in FIG. 13A; it exhibited many of the IPC patterning defects observed with GMR>dCsk-IR. Cell death was consistently observed within clones at early developmental stages, accounting for loss of these clones in the adult (data not shown). Genotypically dCsk cells that are surrounded by normal cells are removed by apoptotic cell death.

Upon closer examination, it was noted that cell death did not occur equally throughout the dCsk mutant patch. First, reducing dCsk in the eye led to a preferential loss of IPCs (FIG. 13B). IPCs have been shown in other contexts to be especially sensitive to cell death stimuli in the young pupa (e.g., Jassim et al., 2003). Second, cell death in FLP-out mediated dCsk clones was observed primarily at the periphery of the clones (data not shown). These clones will eventually die away and presumably as peripheral cells are removed, newly peripheral cells sequentially die. This selective death at the clonal boundary suggests that removal of dCsk boundary cells is due to a short-range, non-autonomous signal from their wild-type neighbors.

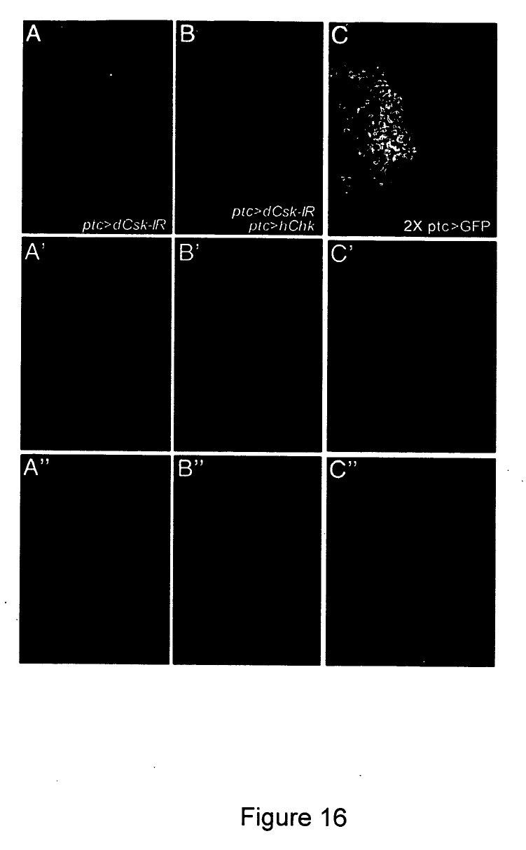

To better understand the loss of discrete dCsk patches, two wing disc-specific drivers with restricted expression domains were utilized: scalloped-GAL4 (sd-GAL4) and patched-GAL4 (ptc-GAL4), which direct expression to the wing pouch and to a few rows of cells along the anterior-posterior (A/P) boundary, respectively (Speicher et al., 1994). Consistent with the observations of clonal tissue, neither driver in combination with UAS-dCsk-IR gave rise to an enlarged wing. Instead, sd>dCsk-IR wings were severely reduced and malformed (FIG. 6C), while ptc>dCsk-IR wings were normal in size but contained morphogenetic defects that included extra wing material in L4 and a defective or missing anterior cross vein (FIGS. 6D and 11B). One possible explanation for these phenotypes is that the wild-type tissue surrounding the transgene expression domain underwent ‘compensatory apoptosis’, a mechanism utilized by epithelia to control tissue size (e.g., de la Cova et al., 2004). However, and consistent with the clone results, TUNEL staining of sd>dCsk-IR and ptc>dCsk-IR wing discs revealed high levels of apoptosis within the scalloped and patched expression domains (FIG. 6G-J). Caspase activation was also increased. As with GMR>dCsk-IR eyes, human CHK was able to rescue ptc>dCsk-IR-mediated phenotypes in the wing (FIG. 16).

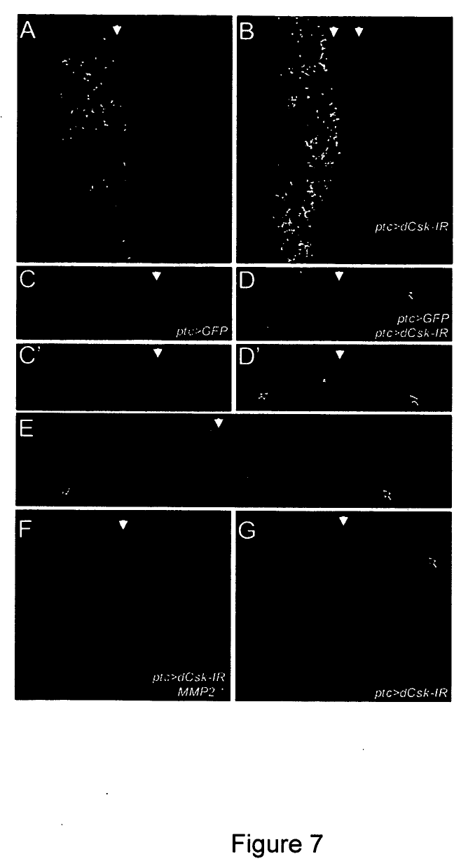

A closer examination of ptc>dCsk-IR wing discs revealed a reproducible pattern of cell death within the patched expression domain. patched-GAL4 directs expression in a gradient in which expression is highest at the A/P boundary and falls away gradually in cells progressively further from the boundary (FIG. 6A). Interestingly, ptc>dCsk-IR wing discs contained apoptotic figures exclusively at the posterior edge of the patched expression domain, that is, in cells directly apposing wild-type cells at the A/P boundary (FIG. 6J). In addition, junction (DEcadherin and P120ctn) and nuclear markers revealed that cells lying within a region encompassing 3-4 cell diameters from the A/P boundary had lost their apical presence and had shifted deeper into the tissue (FIG. 7B); neighboring wild-type cells then contact more anterior dCsk-IR cells to retain apical integrity. This region is notable as the only domain at which a sharp difference in dCsk levels is expected. More anterior cells within the ptc domain showed little or no detectable cell death, suggesting that neighboring cells with only slightly differing levels of the dCsk-IR transgene do not provide a death cue. Consistent with this view, no ectopic apoptosis was observed when utilizing the driver optomotor blind-GAL4 (omb-GAL4, Grimm and Pflugfelder, 1996), which directs a double gradient of expression that fades away both anterior and posterior from the A/P boundary (FIG. 6F).

ptc>dCsk-IR cells were also marked with GFP (ptc>dCsk-IR; ptc>GFP) to follow their fate. Numerous GFP-positive cells were found beneath wing epithelium, including many that were observed to cross compartment boundaries and re-localize to the posterior compartment, separating from the main body of ptc>dCsk-IR cells in the anterior compartment. Based on their rounded morphology, condensed chromatin, and expression of activated caspases these displaced cells were undergoing apoptotic death. Optical sections perpendicular to the epithelial plane confirmed that these ptc>dCsk-IR ‘boundary cells’ dropped beneath the epithelium and migrated across the A/P axis; cells migrated furthest along the folds of the wing disc, where they followed typically along basal channels (FIGS. 7D, 7D′).

Implications for Mechanisms of Metastasis

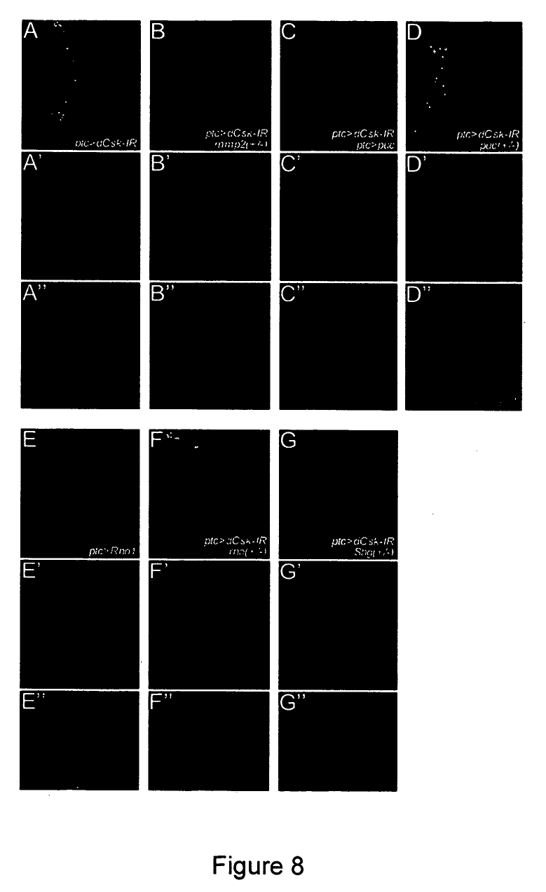

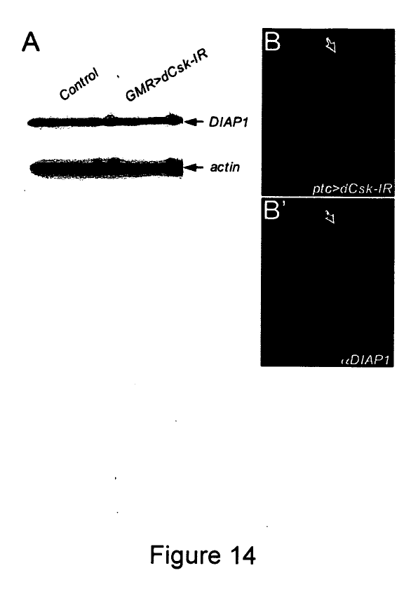

Activation of Src in mammalian cell culture models has been previously shown to induce cell motility in vitro along Matrigel-coated surfaces (e.g., Hauck et al., 2002), and a hallmark of metastatic cells is the expression of proteins required for the degradation of extracellular matrix (ECM) such as matrix metalloproteases (e.g., Minn et al., 2005). The migration of ptc>dCsk-IR ‘boundary cells’ through the basal ECM is reminiscent of this type of cell motility. In fact, halving the genomic dose of matrix metalloproteinase II (MMP2) suppressed the migratory behavior of these ptc>dCsk-IR cells (FIG. 7F) as well as associated cell death (FIG. 8B). Coexpression of the MMP inhibitor TIMP (Page-McCaw et al, 2003; Wei et al, 2003) gave rise to a similar phenotype. Diap1 protein levels were reduced within ptc>dCsk-IR boundary cells compared to control discs (FIG. 14), suggesting that dCsk cells are eliminated by a mechanism that reduces their Diap1 protein levels when juxtaposed with normal cells. The long distances traveled by ptc>dCsk-IR boundary cells and the requirement of MMP2 for this translocation suggest that the movement of cells was not a direct consequence of apoptosis and eventual phagocytic engulfment by their neighbors. Consistent with this view, inducing apoptosis by strong miss-expression of GFP to toxic levels throughout the patched domain did not lead to translocation of these cells (FIG. 16C). This result indicates that the migratory behavior of dCsk cells are likely due to effects that are independent of apoptotic death.

A Role for dJnk and Rho Signaling

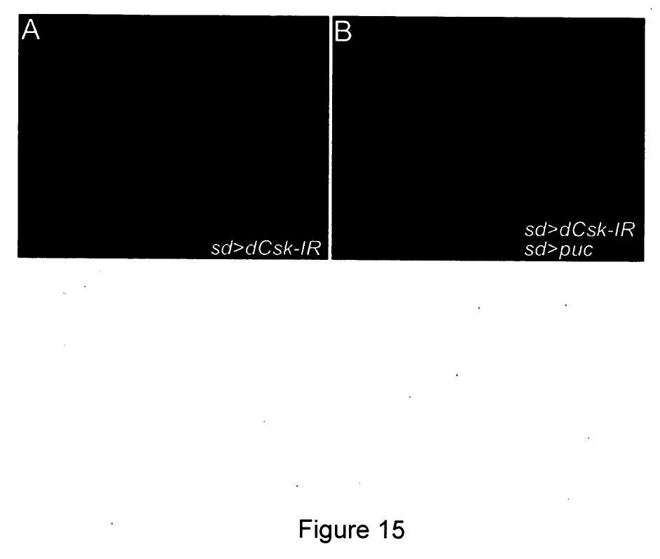

The behavior of dCsk cells presented several differences that distinguished them from cells mutant for salvador, archipelago, hippo, and lats/warts. Instead, dCsk cells shared several features with cells mutant for the tumor suppressor scribble. In particular, scribble mutant cells overgrow in a homotypic environment (Bilder et al., 2000), but are eliminated by JNK-dependent apoptosis when in a clonal patch (Brumby and Richardson, 2003). To determine if activation of the Jnk ortholog dJnk/Basket was required for the removal of dCsk cells at boundaries, dCsk and puckered (ptc>dCsk-IR; ptc>puc) were coexpressed. The puc locus encodes a dJnk-specific phosphatase that provides feedback inhibition to specifically repress dJnk activity (Martin-Blanco et al., 1998; McEwen and Peifer, 2005). By itself, ptc>puc showed no phenotype, but in combination with dCsk-IR, expression of puc prevented apoptosis within the boundaries of the patched and scalloped domains (FIGS. 8C and 15), indicating that boundary-dependent dCsk cell death required dJnk activity. Interestingly, co-expression of puc also rescued the spreading of ptc>dCsk-IR cells away from the boundary. A similar rescue was obtained by the co-expression of a dominant negative dJnk isoform. Conversely, both the apoptotic death and the spreading of dCsk-IR cells were strongly enhanced by halving the genomic dose of puc (ptc>dCsk-IR; puc+/−; FIG. 8D). Together, these results suggest a model in which normal cells signal to dCsk neighbors, promoting migration and death by activating dJnk signaling These two functions have been linked to dJnk in other contexts: dJnk activation in the wing disc results in apoptosis (e.g., Adachi-Yamada et al., 1999), and it is associated with migratory behavior during dorsal closure, disc eversion, and wound healing (Pastor-Pareja et al., 2004; Ramet et al., 2002; Stronach and Perrimon, 1999). This requirement for dJnk signaling raises two essential questions: how is dJnk signaling activated, and why are cells at the posterior boundary of ptc>dCsk-IR uniquely affected?

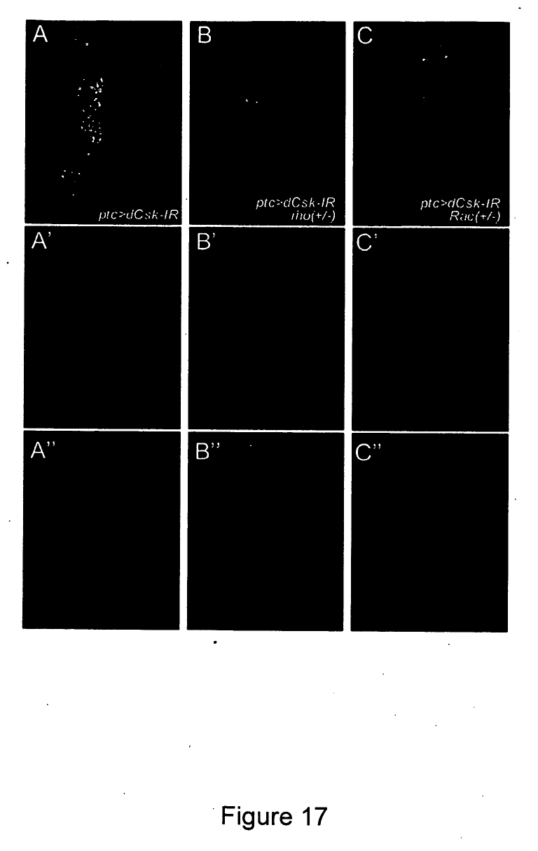

The Drosophila TNF-α ortholog Eiger was considered, as a well-demonstrated activator of the dJNK pathway (Igaki et al., 2002; Moreno et al., 2002b). However, reducing Eiger activity by (i) removing a genomic copy (Eiger+/−) or (ii) RNA-interference-mediated reduction of Eiger or its receptor Wengen/dTNFR did not significantly modify the phenotype of ptc>dCsk-IR. The small GTPases of the Rho family are also activators of JNK (Lim et al., 1996). Previous work has demonstrated that miss-expression of Rho1 results in epithelial exclusion and invasive behavior of wing disc epithelial cells (Speck et al., 2003). ptc>Rho1 cells also released from the epithelium and underwent apoptosis (FIG. 8E) in a manner indistinguishable from discrete loss of dCsk. Furthermore, halving the genomic dose of Rho1 dramatically suppressed both the migration and apoptosis of ptc>dCsk-IR cells (FIGS. 8F and 17B), whereas it did not affect the phenotypes resulting from a broad loss of dCsk. This effect may be specific for Rho1: a similar reduction in the genomic complement of three other members of the Rho family, Rac1, Rac2 and Mtl, did not suppress the ptc>dCsk-IR phenotype, even if reduced simultaneously (FIG. 17C). Taken together, these results indicate that Rho1 acts as a positive mediator of the dCsk signal in boundary cells.

dE-cadherin and p120ctn Mediate Cell Signaling

Src is thought to mediate signaling from a variety of sub-cellular compartments including adherens junctions. ptc>dCsk-IR cells along the A/P boundary initially lost components of their zonula adherens before becoming basally excluded (FIG. 7B). The Drosophila zonula adherens is an apical junction that is functionally equivalent to the mammalian adherens junction, sharing most of its proteins including cadherins, catenins, and also regulation by Src (reviewed in Gumbiner, 2005); for example, alterations in E-cadherin levels has been linked to tumor maturation (e.g., Perl et al, 1998). As described above, cadherin-dependent adhesion was reduced in dCsk mutant tissue, suggesting that the cadherin complex might play a role in the recognition and removal of dCsk cells. Significantly, halving the genomic dose of dE-cadherin by utilizing the null allele ShgR69 suppressed both the migratory and apoptotic phenotypes of ptc>dCsk-IR cells at the boundary (FIG. 8G). RNAi-mediated reduction of dE-cadherin levels had similar results. These results indicate that dE-Cadherin may be part of a signaling network that directs the removal of dCsk boundary cells.

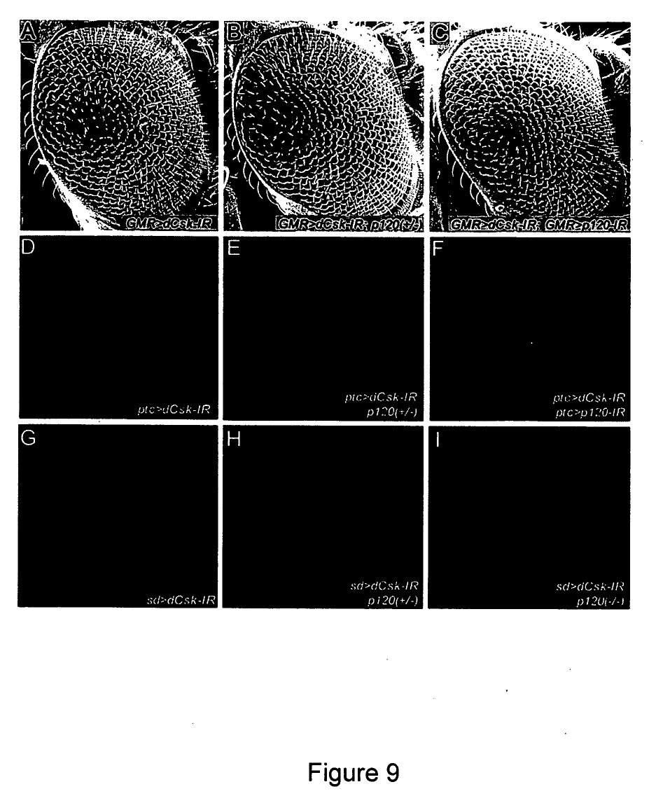

One candidate to provide a link between dCsk, Rho1 signaling, and dE-Cadherin is Drosophila p120-catenin (dP120ctn). Mammalian-P120ctn was one of the first Src substrates identified (Reynolds et al., 1989), although the significance of P120ctn phosphorylation and whether it plays a role in Src-mediated transformation is not clear (reviewed in Reynolds and Roczniak-Ferguson, 2004). Unexpectedly for a core component of the adherens junctions—and in contrast to its vertebrate counterpart—p120ctn is not required for viability in C. elegans or Drosophila (Myster et al., 2003; Pettitt et al., 2003). Also, a mutant dE-cadherin that was unable to bind dP120ctn could nevertheless rescue the lethality of wild-type dE-cadherin (Pacquelet et al., 2003). Thus, the biological role of the only member of the p120ctn family in invertebrates and its role in mediating Src activity in vertebrates has remained an open question. Strikingly, reducing dP120ctn activity by removing a functional genomic copy using the null allele dP120ctn308 led to a significant suppression of the apoptosis and migration phenotypes of cells expressing dCsk-IR within either the ptc or the sd domains (FIGS. 9E and 9H).

Removing both dP120ctn genomic copies led to a still stronger suppression of sd>dCsk-IR (FIG. 91). A similar suppression was observed when dP120ctn activity was reduced by RNA interference (FIG. 9F). These results demonstrate that p120ctn is required to mediate the defects observed at dCsk clonal boundaries.

Similar to Rho1, the effects of reducing p120ctn activity when dCsk was reduced broadly gave a different outcome: reducing dP120ctn activity through RNA-interference or removal of one functional genomic copy failed to modify the phenotype of GMR>dCsk-IR eyes (FIGS. 9B and C). This data is consistent with the view that p120ctn specifically mediates the effects of discrete rather than broad loss of dCsk, once again emphasizing the special nature of cells at clonal boundaries.

Csk family kinases encode critical negative regulators of Src family kinases (SFKs). The present disclosure demonstrates that Drosophila dCsk is a vital negative regulator of growth and proliferation. Loss of dCsk activity leads to overgrowth of multiple tissues and this overgrowth requires the functions of Src-Btk, JNK, and STAT signal transduction pathways. dCsk has been linked to the Lats tumor suppressor (Stewart, 2003). Together, these results provide support for the long suspected role of human Csk kinases as tumor suppressors.

Partial reduction of Src64B, Src42A, or Btk29A activity suppressed the dCsk phenotype, providing functional data to support the view that the dCsk-mediated overgrowth phenotype results from inappropriate activation of the Src-Btk signal transduction pathways. Mutations in Btk29A more strongly suppressed dCsk than either Src42A or Src64B mutations, perhaps reflecting that (i) Src paralogs act redundantly to each other in Drosophila as in mammals and (ii) that Btk29A has previously been shown to act downstream of SFKs in flies and in mammals. The results provide in vivo evidence that Tec-Btk family kinases are critical to SFK-mediated proliferation and suggest that partial reduction of Tec-Btk kinase activity could reduce proliferation in other cellular contexts in which overgrowth is driven by hyperactivated SFKs, such as in colon tumors.

Using a loss-of-function approach to identify effectors that mediate the dCsk overgrowth phenotypes failed to implicate some of these pathways in dCsk function. For example, SFKs upregulate the SOS-Ras-ERK pathway in multiple tissue culture studies and Drosophila overexpression models. However, although dRas1 signaling is active throughout retinal development, reduced dEGFR, drk (GRB2), Sos, and Jra (c-jun) gene dosage failed to affect the dCsk phenotype and dCsk failed to modify a hypermorphic allele of dEGFR. Levels of doubly-phosphorylated and activated ERK appeared unaltered in dCsk^{-/-} tissue (data not shown). These data argue that not every signal transduction pathway implicated in SFK tissue culture models necessarily functions as predicted within a developing epithelial tissue.

The genetic studies emphasized the importance of two signaling pathways in dCsk and SFK function. Phenotypic and FACS analysis established that reduced JNK (bsk) function suppressed the phenotypes and cell cycle defects caused by loss of dCsk. These results support studies indicating that JNK functions downstream of the Src-Btk pathway in Drosophila and mammalian tissue culture cells. Components of the JNK pathway are required for Src-dependent cellular transformation, but the exact role of JNK in these cells is unknown. Importantly, the data disclosed herein show that the JNK pathway mediates proliferative responses to Src signaling in vivo, but further work will be needed to precisely understand its mechanism.

dCsk proved a negative regulator of Jak/Stat signaling. For example, dCsk mutant tissues up-regulated Stat92E protein, a hallmark of Jak/Stat activation in Drosophila. Stat92E, the sole Drosophila STAT ortholog, is most similar to mammalian STAT3. In mammalian cells, Src directly phosphorylates and activates STAT3, and STAT3 function and activation are required for Src transforming activity. Conversely, overexpression of Csk blocks STAT3 activation in v-Src transformed fibroblasts. However, the physiological significance of these interactions within developing epithelia has remained unclear.

dCsk; Stat92E double mutant clones demonstrated that loss of STAT function severely reduced Src-dependent overgrowth and promoted apoptosis of mutant tissue. dCsk^{-/-}; Stat92E^{-/-} EGUF adult eyes are strikingly similar to phenotypes caused by over-expression of Dacapo, the fly ortholog of the cdk inhibitor p21, and PTEN, a negative regulator of cell proliferation and growth. Importantly, removing Stat92E function in dCsk mutant tissue led to a synthetic small eye phenotype and did not simply rescue the dCsk^{-/-} proliferative phenotype. This outcome distinguishes Stat92E from mutations in Src64B, Btk29A, or bsk, which rescued dCsk-mediated defects toward a normal phenotype. The loss of tissue in dCsk^{-/-}; Stat92E^{-/-} clones indicates that Src signaling provokes apoptosis and blocks normal proliferation in the absence of Stat92E function. Consistent with this interpretation, reduced Btk29A function rescued the dCsk^{-/-}; Stat92E^{-/-} EGUF phenotype to a more normal phenotype, demonstrating that the reduced growth and apoptosis of the dCsk^{-/-}; Stat92E^{-/-} tissues is indeed Src-pathway-dependent.

The data disclosed herein suggest the existence of a Src-dependent pro-apoptotic and anti-proliferative pathway that is normally suppressed by STAT. One possible component of this pathway is JNK given that JNK signaling is an important activator of apoptosis in both flies and mammals. Perhaps Src-dependent hyperactivation of Bsk (JNK) in dCsk^{-/-}; Stat92E^{-/-} tissue contributes to cell death in the absence of proliferative and/or survival signals provided by Stat92E. However, a number of other candidate pathways may also mediate this response. The further characterization and identification of these pathways may have important implications for interceding in Src-mediated oncogenesis.

Together, these observations indicate that, in tissue that contains hyperactive Src or reduced Csk, blocking STAT function is sufficient to decrease proliferation and trigger apoptosis in the absence of any further mutations or interventions. Reduced STAT3 function can promote apoptosis within breast and prostate cancer cells that show elevated SFK activity, but the molecular pathways driving apoptosis in these cells are unknown (Garcia, 2001 #4514; Mora, 2002 #4524). These cells may require survival signals provided by STAT3 to counteract apoptosis due to chromosomal abnormalities or other defects. Alternatively, these cells may die because of pro-apoptotic signals provided by hyperactive SFKs in the absence of STAT3 function. The data disclosed herein argue that the latter may be true, which suggests the intriguing possibility that therapeutic blockade of STAT function in tumors with activated Src may actively provoke Src-dependent apoptosis and growth arrest in tumor tissues.

### EXAMPLES

Aspects of the present teachings may be further understood in light of the following examples, which should not be construed as limiting the scope of the present teachings in any way.

### Example 1

### dRet: Targeted Expression and Association with Cancer

To mimic the MEN2B mutation, a single methionine-to-threonine point mutation was engineered into a full-length dRet cDNA at codon 1007 (analogous to position 918 within human hRet subdomain VIII). To mimic the human MEN2A mutation, the cysteine at position 695 in dRet was altered to an arginine (C695R) that is in a position most analogous to hRet 634, one of the most commonly mutated sites in MEN2A patients. All mutated fragments were sequenced and returned to the original. dRet clone to produce dRet^{MEN2A} and dRet^{MEN2B}. dRet, dRet^{MEN2A} and dRet^{MEN2B} were then fused 3′ to a GMR promoter construct that directs expression exclusively and at high levels in the eye (Moses and Rubin, 1991); stable transgenic lines were then created by standard protocol to yield GMR-dRet, GMR-dRet^{MEN2A}, and GMR-dRet^{MEN2B}.

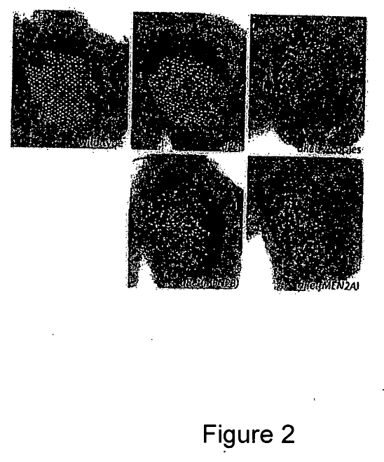

Targeting MEN2A-analogous and MEN2B-analogous forms of dRet by standard methods for expression within the developing Drosophila retina also led to an overgrowth phenotype that mimics aspects of the human MEN2A and MEN2B diseases. FIG. 2 presents typical examples from each phenotype. A wild-type eye is included for reference: note how the ommatidia are organized into smooth rows (FIG. 2, top left).

Expression of one copy of the GMR-dRet construct gave either a normal phenotype or some led to a mildly roughened eye (FIG. 2, top row, center panel). Two copies of the same transgene in all GMR-dRet insertions led to a strongly roughened eye (FIG. 2, top row, right panel). As indicated in the lower panels, the phenotypes of GMR-Ret^{MEN2A} and GMR-Ret^{MEN2B}, designed to mimic MEN2A and MEN2B are more severe both as single copies (bottom row) and as multiple copies (not shown). They typically contain fused ommatidia with severe patterning defects. The variable number of ommatidia suggest alterations in cell proliferation and cell death, aspects commonly observed in human tissue with constituent tumors.

### Example 2

### Screening of ZD6474

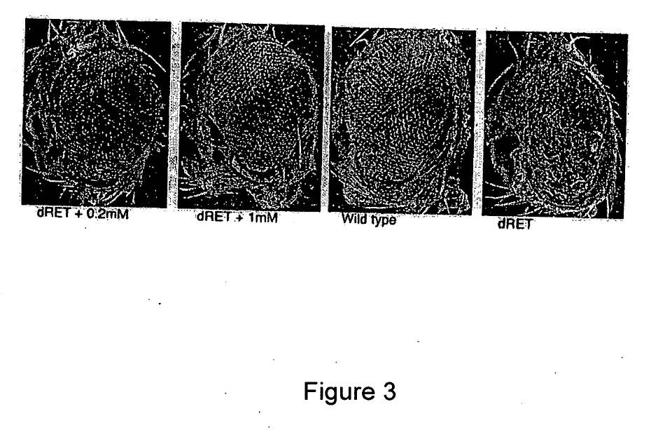

A candidate therapeutic compound identified as ZD6474, obtained from AstraZeneca International, had previously been tested and found to reduce Ret activity in a tissue culture model (Carlomagno et al., 2002); this drug also shows some efficacy for VEGF-class receptors (Ciardiello et al., 2004; Ciardiello et al., 2003; Glade-Bender et al., 2003; Hennequin et al., 2002; Wedge et al., 2002). FIG. 3 illustrates in part the results of screening compound ZD6474 according to the screening methods of the present invention. Screening demonstrated the ability of ZD6474 to strongly inhibit the severity of the rough eye phenotype of both dRet and dRet^{MEN2B}, indicating that the overgrowth and phenotypic defects were ameliorated. The panels in FIG. 3 demonstrate that the ZD6474 compound can rescue the dRet^{MEN2B} phenotype in a concentration-dependent fashion. Overall, toxicity was observed at concentrations at and above 2.5 mM, and at least partial rescue was observed with doses as low as 0.04 mM. Therefore, the estimated therapeutic index (ratio of concentrations that are toxic to Drosophila to concentrations that reduce the retinal phenotype is 2.5/0.04=31.

These data demonstrate that using the screening methods of the present invention, candidate therapeutic compounds can be screened for ability to reduce inhibit or prevent the effects of oncogenic forms of proteins. These results therefore support the application of the Drosophila screening method to the identification of candidate compounds, other drugs or genes that might ameliorate overgrowth and other defects in tissues that contain abnormal biochemical activity. It is recognized that this approach of screening altered Drosophila with compounds in the microwell-based approach described above can be utilized in other Drosophila models of animal disease, and particularly human disease.

### Example 3

### Drosophila Ortholog of C-terminal Src Kinase (Csk) Regulates Cell Growth and Proliferation Through Inhibition of the Src, JNK, and STAT Pathway

The Src family cytoplasmic tyrosine kinases play important roles in a wide variety of cellular processes including proliferation and differentiation. Their major regulation is by C-terminal Src kinase (Csk), which encodes a negative regulator of Src tyrosine kinase signaling. The Drosophila ortholog of Csk, dCsk, functions as a tumor suppressor: dCsk mutants demonstrated increased body size and over-proliferation of adult tissues. Src family kinases regulate multiple cellular processes including proliferation and oncogenesis. Csk encodes a critical negative regulator of Src family kinases. The Drosophila Csk ortholog, dCsk, is demonstrated to function as a tumor suppressor: dCsk mutants display organ overgrowth and excess cellular proliferation. Results of genetic analysis revealed that the dCsk phenotype depends primarily on activation of the Src, Jun kinase, and STAT signal transduction pathways. Blockade of Stat92E function in dCsk mutants severely reduced Src dependent overgrowth and activated apoptosis of mutant tissue. These data confirm work in mammalian tissue culture that links Src transforming activity to STAT function and provides an in vivo model for the interplay of Csk and Src kinases.

### Example 4

### dCsk Fly Stocks and Genetics

UAS-dCsk-IR flies were generated from a fragment from the dCsk gene of 590 bp amplified by PCR from dCsk cDNA with the following primers:

TGTCTTCACCAGCAAGCATC (SEQ ID NO: 8)
CTCCCTTGCTGACTCCTCAC (SEQ ID NO: 9)

The fragment was cloned as an inverted repeat into the pWIZ vector (Lee and Carthew, 2003), injected in yw; Δ2-3 Ki embryos by transformation protocols standard to the art, and isogenic balanced stocks were generated by standard genetic crosses. A UAS-dCsk-IR transgene inserted on the X chromosome was used for all experiments.

Human CHK cDNA sub-cloned from pcDNA3 into pUAST was used to create UAS-hChk flies. dCsk and hCHk show low sequence conservation at the DNA level, so hCsk is not an expected target of dCsk-IR.

dCsk mitotic or RNAi (inverted repeat) FLP-out clones were made by the FRT/FLP technique. A one hour 37° C. heat shock was used to induce hs-FLPase in 0-48 hour old embryos. To create EGUF (Stowers and Schwartz, 1999) clones, flies with the genotype yw:ey-gal4, UAS-FLP/+; FRT82B GMR-hid I(3)CL-R/FRT82B dCsk were generated by standard genetic crosses. Cultures were in standard molasses media at 25° C. unless otherwise stated.

### Example 5

### Effects of Reducing dCsk Expression in the Retina

To explore dCsk function, dCsk transcript was targeted for reduction by RNA interference (RNAi) through the use of an inverted repeat (IR)—containing transgene. Use of the GAL4/UAS system (Brand and Perrimon, 1993) provided temporal and spatial control of expression.

For scanning electron microscopy, adult flies were fixed in 95% ethanol, critical point dried, sputter coated, and viewed with a Hitachi S-2600H scanning electron microscope. FIG. 4 shows the micrographs from adult eyes for (A) wild-type, (B) dCsk^{c04256} (EGUF), (C)GMR>dCsk-IR and (D) GMR>dCsk-IR; GMR>hChk flies. The GMR>dCsk-IR eyes showed an enlarged and rough eye phenotype (FIG. 4C), and this phenotype was suppressed by co-expression of the human ortholog CHK (FIG. 4D), indicating that human Chk can at least partially replace dCsk function.

For immunofluorescence, pupal retinas from staged animals, and wing and eye imganinal disks from wandering third larvae, were dissected in PBS and processed. (see Brachmann et al., 2000). Anti-Armadillo staining was used to visualize cells in retinas 42 hours after puparium formation (APF). One ommatidium was pseudo-colored to illustrate the cell types found at the apical surface (FIG. 4E). Four cone cells (c) and two primary pigment cells (1°) comprise the ‘ommatidial core’; eight photoreceptor neurons lie beneath and are not seen. The interommatidial lattice is composed of six secondary pigment cells (2°), three tertiary pigment cells (3°) and three bristle groups (b).

Reduction in dCsk activity resulted in mis-patterning of the interommatidial lattice (FIG. 4F-G). In FIGS. 1F and 1G, higher brightness was required to visualize cell outlines because Armadillo staining in IPCs (but not photoreceptors) was reduced. FIG. 4G shows a common phenotype: 3°s normalcy contact three 1°s, but failed to retain their proper niche in GMR>dCsk-IR retinas (compare to inset in FIG. 4E). Co-expression of dE-cadherin rescued the Armadillo staining and patterning defects (FIG. 4H). Note that GMR-reaper eyes (FIG. 4I) are small and rough due to ectopic cell death. This phenotype was in turn partially suppressed in the presence of GMR>dCsk-IR (FIG. 4J).

TUNEL was performed using the TMR-Red In situ Cell Detection Kit (Roche Diagnostics). Images were captured using either a Zeiss Axiophot or a Leica confocal microscope. Images were further processed with Adobe Photoshop to improve contrast and light balance. Investigation of the effects of dCsk interference showed that apoptosis due to mis-expression of the Reaper and Hid genes was suppressed when dCsk activity was reduced. (FIGS. 4K and 4L).

### Example 6

### Live Imaging

Transgenic animals were staged to 27 hr APF (24% pupa life at 25° C.), the pupal case removed in the head area, and the animal placed with the eye region pressed against a coverslip. Snapshots were taken every 15 minutes, with temperature and humidity controlled, utilizing a fluorescence microscope. The control genotype did not give a discernible adult eye phenotype, and the analysis of late pupa retinas showed very infrequent extranumerary secondary pigment cells and bristle defects due to the GAL4 and αCatenin-GFP transgenes (data not shown).

### Example 7

### In Situ Visualization of Developing dCsk Retinas

FIG. 5 panels show snapshots from a supplementary movie taken at the indicated hours APF as indicated in each panel of FIG. 5. FIG. 5A-D shows αGMR>Catenin Catenin-GFP control retina. Hours after puparium formation (APF) are indicated for each panel. Red pseudo-coloring highlights cells that were removed by programmed cell death during this time-course. Red pseudo-coloring highlights cells that were removed by programmed cell death during this time-course. Green pseudo pseudo-coloring indicates a cell that pushed stably into a vertex niche to become a 3° pigment cell. By 27 hr APF, IPCs moved from multiple cell layers to single file around 1°s (FIG. 5A). At this stage, cells began to push between each other to establish first (i) stable 3°s at the vertices and then (ii) the 2° array. Concurrently, excess cells were removed by programmed cell death (FIGS. 5B-D). 5E through L show snapshots from a supplementary movie of a GMR>dCsk-IR; GMR>αCatenin-GFP eye. Blue pseudo-colored bristle cells moved past IPCs to contact each other; adjacent bristles are not seen in normal eyes. Orange and green pseudo-colored cells were initially normally patterned; these cells failed to hold this position and instead continued to move past each other to form aberrant IPC clusters. One cell transiently had an extremely reduced, darkened apical profile normally suggesting it would apoptose (see arrow in inset from FIG. 7J) but this cell later recovered and established a niche in the interommatidial lattice. Unlike for the controls, programmed cell death was never observed in movies from this experimental genotype, even at the stages when programmed cell death is normally maximal, confirming that loss of programmed cell death is a primary cause of ectopic interommatidial cells in dCsk eyes. Although most IPCs moved initially into their proper niches, a striking inability of these cells to hold their positions was observed. For example, a cell that contacts three 1°s normally settles stably as a 3°, but the experimental genotype cells move into and then but of the 3° niche. Cells were observed to move several cell lengths away, gliding over other cells to form abnormal ‘piles’ of cells (y, e.g., green cells in FIGS. 5E-L). Often, these too robust, aberrant cell movements disorganized already already-patterned areas (e.g., orange cells in FIGS. 5E-L).

### Example 8

### Wing Size Measurements

Wings from female flies were photographed and wing area dimensions measured using Image J software (NIH). A minimum of 20 wings from different animals was measured per genotype. For statistical analysis a two-tailed student's t-test was used. Results represent percentage size over control genotype, ±S.E.M. p≦0.05. FIG. 6 shows wing phenotypes for dCsk cells. (A) is a schematic representation of the expression domains for the 765, patched (ptc), scalloped (sd) and optomotor blind (omb) promoters in the wing disc. Anterior is left and dorsal up. (B) shows wings from 765>dCsk-IR adults. Control: 100±0.5%, n=35. Experimental: 107.2±0.4%, n=37 (p23 0.05). 5C shows escapers with sd>dCsk-IR adult wings were severely malformed and reduced in size. FIG. 6D shows escapers with ptc>dCsk-IR adult wings were normal in size. Control (ptc-gal4/+): 100±0.6%, n=27. Experimental: 98.7±1.7%, n=33.

FIGS. 6(E) through (J) show wing discs stained with TUNEL (red) and anti-β-galactosidase staining (green). Asterisks mark ptc expression in the overlying peripodial membrane and the arrows mark the anterior-posterior boundary. FIGS. 6(E-G) show results from TUNEL analysis of wing discs showing wing phenotypes for dCsk cells. FIGS. 6(E) and (F) show that 765>dCsk-IR; 765>lacZ and omb>dCsk-IR; omb>LacZ discs displayed very few TUNEL-positive cells, while sd>dCsk-IR; sd>LacZ discs contained TUNEL positive cells that were concentrated primarily near the edges of the expression domain. FIGS. 6(G-H) ptc>dCsk-IR; ptc>LacZ wing discs exhibited high levels of TUNEL-positive cells primarily at the posterior edge of the expression domain, next to the anterior/posterior boundary (arrow) (FIGS. 6I, J). (K-L) show how dCsk mutant cells are eliminated from normal tissue. (K) shows a wing disc with the genotype hs-FLP; FRT82BdCskSO30003/FRT82Bubi-GFP(nls). (L) shows retina at 42 hr APF with the genotype hs-FLP; FRT82BdCskj1d8/FRT82Bubi-GFP(nls). The asterisk marks exogenousmigratory cells laying on top of the retina that show autofluorescence.

Wing phenotypes unveiled a microenvironment-specific behavior of dCsk cells. Schematic representation of the expression domains for the 765, patched (ptc), scalloped (sd) and optomotor blind (omb) promoters in the wing disc are shown in FIG. 6A. Anterior is left and dorsal up. Examination of ptc>dCsk-IR wing discs revealed a reproducible pattern of cell death within the patched expression domain. patched-GAL4 directed expression in a gradient in which expression was highest at the A/P boundary and fell away gradually in cells progressively further from the boundary.

Wings from 765>dCsk-R adults were larger than controls (765-gal4/+) (FIG. 6B). Control: 100±0.5%, n=35. Experimental: 107.2±0.4%, n=37 (p≦0.05).

Escapers with sd>dCsk-R adult wings were severely malformed and reduced in size. (FIG. 6D). Escapers with ptc>dCsk-IR adult wings were normal in size. Control (ptc-gal4/+): 100±0.6%, n=27. Experimental: 98.7±1.7%, n=33.

### Example 9

### dCsk Mutant Cells are Eliminated from Normal Tissue

Mitotic clones were induced in the early embryo by administering a 1 hour heat shock at 37° C., 0-48 hr after egg deposition (AED). FIG. 6K shows wing disc with the genotype hs-FLP; FRT82BdCskSO30003/FRT82Bubi-GFP(nls), and FIG. 6L shows retina at 42 hr APF with the genotype hs-FLP; FRT82BdCskj1d8/FRT82Bubi-GFP(nls). In both cases, lack of fluorescence marks mutant tissue (dCsk−/dCsk−), weak fluorescence marks the background heterozygous tissue (ubi-GFP, dCsk+/dCsk−) and the stronger fluorescence marks the “twinspot” wild-type tissue (ubi-GFP, dCsk+/ubi-GFP, dCsk+). The asterisk marks exogenous migratory cells laying on top of the retina that show autofluorescence.

### Example 10

### Boundary dCsk Cells Lose their Apical Profile, are Basally Excluded and Migrate Across the ECM

FIG. 7 shows position and profile of boundary dCsk cells. (A-B) show confocal projections from wing discs with the genotypes ptc>p120ctn-GFP; ptc>RFP(nls) (A) and ptc>p120ctn-GFP; ptc>RFP(nls); ptc>dCsk-IR (B). Arrows mark the anterior/posterior boundary in all panels. (C and D) show phalloidin staining from wing discs from animals with the genotypes ptc>GFP(C) and ptc>GFP; ptc>dCsk-IR (D). (C and D) show confocal sections from an apical view, (C′ and D′) show optical sections in apical/basal, anterior/posterior planes. Green arrows point to basally excluded cells that migrated away from the boundary presumably trough the ECM. (E) is a schematic drawing derived from panel D′ to clarify cell positions. (F-G) are confocal projections of wing discs from animals with the genotypes ptc>dCsk-IR; MMP2EY089421+(F) and ptc>dCsk-IR (G).

dCsk-IR boundary cells had no apical profile and dP120ctn protein was de-localized. This effect was not specific for dP120ctn since it was also observed for membrane-targeted RFP (myr-RFP, data not shown). Phalloidin staining from wing discs from animals with the genotypes ptc>GFP and ptc>dCsk-IR were marked with GFP kptc>dCsk-IR; ptc>GFP) to follow their fate. FIGS. 7C and D show confocal sections from an apical view, C′ and D′ show optical sections in apical/basal, anterior/posterior planes Green arrows point to basally excluded cells that migrated away from the boundary presumably through the ECM.

FIG. 7E shows a schematic drawing derived from panel D′ to clarify cell positions. Cells leave near the anterior/posterior boundary and move in all directions.

FIGS. 7F and G show confocal projections of wing discs from animals with the genotypes ptc>dCsk-IR; MMP2EY089421+(FIG. 7E) and ptc>dCsk-IR (FIG. 7F). Reducing MMP2 genomic copy number strongly reduced cell migration. Halving the genomic dose of matrix metalloproteinase II (MMP2) suppressed the migratory behavior of ptc>dCsk-IR (FIG. 7F) as well as associated cell death (FIG. 8B).

### Example 11

### Rho1, JNK, and dE-cadherin Activity are Required for the Removal of dCsk Cells

To determine if activation of the Jnk ortholog dJnk/Basket was required for the removal of dCsk cells at boundaries, dCsk and puckered (ptc>dCsk-IR; ptc>puc) were co-expressed. The puc locus encode a dJnk-specific phosphatase that provides feedback inhibition to specifically repress dJnk activity (Martin-Blanco et al., 1998; McEwen and Peifer, 2005).

FIG. 8 shows anti-cleaved Caspase-7 staining (red) from wing discs with the genotypes ptc>dCsk-IR; ptc>GFP (A), ptc>dCsk-IR; mmp22353/− (B), ptc>dCsk-IR; ptc>GFP; ptc>puc (C), ptc>dCsk-IR; ptc>GFP; pucE69/+(D), ptc>rho1; ptc>GFP (E), ptc>dCsk-IR; ptc>GFP; rho172O+(F) and ptc>dCsk-IR; ptc>GFP; shgR69/+(G). The brackets in (A), (D), and (E) show the position of apoptotic GFP-positive cells that have re-located into the posterior compartment.

In combination with dCsk-IR, expression of puc prevented apoptosis with the boundaries of the patched and scalloped domains (FIG. 8C), which indicated that boundary-dependent dCsk cell death required dJnk activity. Both apoptotic death and the spreading of dCsk-IR cells were strongly enhanced by halving the genomic dose of puc (ptc>dCsk-IR; puc^{+/-}) (FIG. 8D).

ptc>Rho1 released from the epithelium and underwent apoptosis in a manner indistinguishable from discrete loss of dCsk (FIG. 8E). Halving the genomic dose of Rho1 dramatically suppressed both eh migration and apoptosis of ptc>dCsk-IR cells, (FIG. 8F), whereas it did not affect the phenotypes resulting from a broad loss of dCsk (data not shown), indicating that Rho1 acts as a positive mediator of the dCsk signal in boundary cells.

Cadherin also affects migration and apoptosis of dCsk boundary cells. Halving the genomic dose of dE-cadherin by utilizing the null allele ShgR⁶⁹ suppressed both the migratory and apoptotic phenotypes of ptc>dCsk-IR cells at the boundary (FIG. 8G).

### Example 12

### p120ctn is Required for the Removal of dCsk Boundary Bells

FIG. 9 shows the effect of p120ctn on the removal of dCsk boundary cells. (A-C) show SEMs from adults eyes with the genotypes GMR>dCsk-IR (A), GMR>dCsk-IR; p120ctn308/+ (B) and GMR>dCsk-IR; GMR>p120ctn-IR (C). (D-H) show anti-cleaved Caspase-7 staining (red) from wing discs with the genotypes ptc>dCsk-IR, ptc>GFP (D), ptc>dCsk-IR; ptc>GFP; p120ctn308/+ (E), ptc>dCsk-IR; ptc>GFP; ptc>p120ctn-IR (F), sd>dCsk-IR (G), sd>dCsk-IR; p120ctn308/+ (H) and sd>dCsk-IR; p120ctn308/p120ctn308 (I).

Reducing dP120ctn activity by removing a functional genomic copy using the null allele dP120ctn³⁰⁸ led to a significant suppression of the apoptosis and migration phenotypes of cells expressing dCsk-IR within either the ptc or the sd domains (FIGS. 9E and 9H). Removing both dP120ctn genomic copies led to a still stronger suppression of sd>dCsk-IR (FIG. 91). A similar suppression resulted when dP120ctn activity was reduced by RNA-interference (FIG. 9F).

RNA interference or removal of one functional genomic copy of dP120ctn failed to modify the phenotype of GMR>dCsk-IR (FIGS. 9B and C). These data together suggest that p120ctn mediates the effects of discrete rather than broad loss of dCsk.

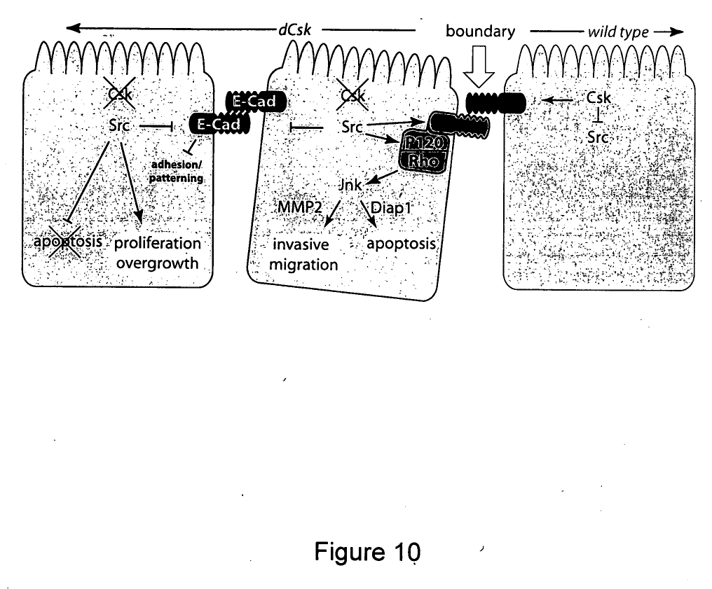

The results described herein support a model for the behavior of dCsk cells. FIG. 10 shows such a model, emphasizing the cellular and molecular differences between dCsk/dCsk and dCsk/wild-type cell boundaries.

### Example 13

### Broad Expression of the dCsk-IR Transgene Phenocopies dCsk Mutants

FIG. 11 shows SEMs of wings in mutants showing that broad expression of the dCsk-IR transgene phenocopies dCsk mutants: (A) actin5C-gal4>dCsk− IR resulted in early pupal lethality. Pupae were approximately 50% longer than actin5C-gal4/+ controls. (B) 765>dCsk-IR wings displayed patterning defects in veins L2, L5 and posterior cross vein (PCV), and ptc>dCsk-IR wings displayed defects including extra vein material in L3 and a defective or missing anterior cross vein (arrow), and (C) shows that anti-Armadillo staining was disrupted in sev>dSrc42A(KD).

### Example 14

### dE-cadherin/Shotgun Co-expression Suppresses the Rough-Eye Phenotype

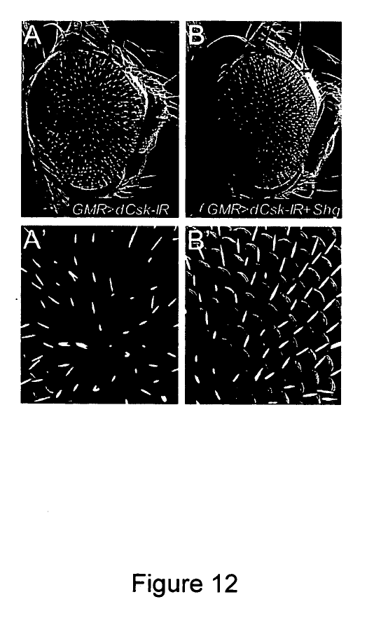

FIG. 12 shows SEMs of adult eyes showing that dE-cadherin/Shotgun co-expression suppresses the rough-eye phenotype observed in GMR>dCsk-IR adult eyes. GMR>GFP was also expressed in (A) as an additional control. Lower panels provide a higher magnification view.

### Example 15

### Comparable Patterning and Cell Number Defects in Other Clones

FIG. 13 shows (A) an example of a rarely obtained dCsk-IR FLP/FRT-mediated clone (hs-FLP; tub>GFP>gal4; UAS-dCsk-IR). The absence of GFP fluorescence (green) marks the mutant clonal tissue. In both (A) and (B), anti-Armadillo staining (red) highlights cell outlines. (B) is a rare retina clone of genotypically dCsk cells at 42 hr APF (genotype: hs-FLP; FRT82BdCskj1d8/FRT82Bubi-GFP(nls)). Absence of GFP fluorescence marked clonal tissue. IPC's were rarely represented in these clones, and the twin spots (bright areas) were invariably far larger. The Armadillo staining at the surface of mutant 1° cell facing the interomedial lattice was found in a different focal plane (arrows). In both (A) and (B), FRT recombination was induced by a 1 s heat shock at 37° C. at 0-48 hr AED.

FIG. 14 shows (A) Western blotting from control (GMR-gal4/+) and GMR>dCsk-IR retinas at 29 hr APF with anti-DIAP1 and anti-Actin antibodies. (B and B′) show anti-DIAP1 antibody staining (red) from a wing disc with the genotype ptc>dCsk-IR; ptc>GFP. The arrow points to the A/P boundary. DIAP1 levels are reduced in cells of patched expression domain adjacent to the boundary.

FIG. 15 shows anti-cleaved Caspase-7 staining from wing discs with the genotypes sd>dCsk-IR (A) and sd>dCsk-IR; sd>puc (B). Expression of puckered suppressed apoptosis of dCsk cells at the boundary of the scalloped expression domain.

FIG. 16 shows anti-cleaved Caspase-7 staining (red) from wing discs with the genotypes ptc>dCsk-IR; ptc>GFP (A) and ptc>dCsk-IR; ptc>GFP; ptc>hChk (B). Co-expression of human Chk rescued dCsk-IR-mediated apoptosis and migration phenotypes. (C) shows enhanced-GFP expressed with two copies of patched (ptc>GFP; ptc>GFP) and raised at 29° C. to increase GAL4-mediated GFP expression. The resulting cell death did not result in extended re-localization of cells from the patched domain into the posterior compartment, uncoupling cell death and cell migration. A few ptc-GFP apoptotic bodies can be seen a short distance into the posterior compartment, presumably due to phagocytosis by their wild-type neighbors.

FIG. 17 shows specificity for Rho1 GTPase activity at dCsk boundary cells. 17(A-C) shows anti-cleaved Caspase-7 staining (red) from wing discs with the genotypes ptc>dCsk-IR; ptc>GFP (A), ptc>dCsk-IR; rho1E310/+ (B) and ptc>dCsk-IR; ptc>GFP; Rac1j11/+, Rac2Δ/+, MtlΔ/+ (C). Similar to rho1⁷²⁰, rho1^{E310} suppressed the phenotype of ptc>dCsk-IR. Simultaneously removing one functional genomic copy in each 4 of the three Drosophila Rac orthologs had no detectable effect.

### OTHER EMBODIMENTS

When introducing elements of the present invention or the preferred embodiments thereof, the articles “a”, “an”, “the” and “said” are intended to mean that there are one or more of the elements. The terms “comprising”, “including” and “having” are intended to be inclusive and mean that there may be additional elements other than the listed elements.

As various changes could be made in the above constructions without departing from the scope of the invention, it is intended that all matter contained in the above description or shown in the accompanying drawings shall be interpreted as illustrative and not in a limiting sense. Indeed, various modifications of the invention in addition to those shown and described herein will become apparent to those skilled in the art from the foregoing description which do not depart from the spirit or scope of the present inventive discovery. Such modifications are also intended to fall within the scope of the appended claims.

### REFERENCES CITED

All publications, patents, patent applications and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present invention. References include:
Acton, D. S., Velthuyzen, D., Lips, C. J., and Hoppener, J. W. (2000). Multiple endocrine neoplasia type 2B mutation in human RET oncogene induces medullary thyroid carcinoma in transgenic mice. Oncogene 19, 3121-3125.
Alberti, L., Borrello, M. G., Ghizzoni, S., Torriti, F., Rizzetti, M. G., and Pierotti, M. A. (1998). Grb2 binding to the different isoforms of Ret tyrosine kinase. Oncogene 17, 1079-1087.
Arighi, E., Alberti, L., Torriti, F., Ghizzoni, S., Rizzetti, M. G., Pelicci, G., Pasini, B., Bongarzone, I., Piutti, C., Pierotti, M. A., and Borrello, M. G. (1997). Identification of Shc docking site on Ret tyrosine kinase. Oncogene 14, 773-782.
Bardelli, A., Longati, P., Gramaglia, D., Basilico, C., Tamagnone, L., Giordano, S., Ballinari, D., Michieli, P., and Comoglio, P. M. (1998). Uncoupling signal transducers from oncogenic MET mutants abrogates cell transformation and inhibits invasive growth. Proc Natl Acad Sci USA 95, 14379-14383.
Besset, V., Scott, R. P., and Ibanez, C. F. (2000). Signaling complexes and protein-protein interactions involved in the activation of the Ras and phosphatidylinositol 3-kinase pathways by the c-Ret receptor tyrosine kinase. J Biol Chem 275, 39159-39166.
Bocciardi, R., Mograbi, B., Pasini, B., Borrello, M. G., Pierotti, M. A., Bourget, I., Fischer, S., Romeo, G., and Rossi, B. (1997). The multiple endocrine neoplasia type 2B point mutation switches the specificity of the Ret tyrosine kinase towards cellular substrates that are susceptible to interact with Crk and Nck. Oncogene 15, 2257-2265.
Bongarzone, I., Vigano, E., Alberti, L., Borrello, M. G., Pasini, B., Greco, A., Mondellini, P., Smith, D. P., Ponder, B. A., Romeo, G., and Pierotti, M. A. (1998). Full activation of MEN2B mutant RET by an additional MEN2A mutation or by ligand GDNF stimulation. Oncogene 16, 2295-2301.
Borrello, M. G., Pelicci, G., Arighi, E., De Filippis, L., Greco, A., Bongarzone, I., Rizzetti, M., Pelicci, P. G., and Pierotti, M. A. (1994). The oncogenic versions of the Ret and Trk tyrosine kinases bind Shc and Grb2 adaptor proteins. Oncogene 9, 1661-1668.
Califano, D., Rizzo, C., D'Alessio, A., Colucci-D'Amato, G. L., Cali, G., Bartoli, P. C., Santelli, G., Vecchio, G., and de Franciscis, V. (2000). Signaling through Ras is essential for ret oncogene-induced cell differentiation in PC12 cells. J Biol Chem 275, 19297-19305.
Carlomagno, F., Melillo, R. M., Visconti, R., Salvatore, G., De Vita, G., Lupoli, G., Yu, Y., Jing, S., Vecchio, G., Fusco, A., and Santoro, M. (1998). Glial cell line-derived neurotrophic factor differentially stimulates ret mutants associated with the multiple endocrine neoplasia type 2 syndromes and Hirschsprung's disease. Endocrinology 139, 3613-3619.
Carlomagno, F., Vitagliano, D., Guida, T., Ciardiello, F., Tortora, G., Vecchio, G., Ryan, A. J., Fontanini, G., Fusco, A., and Santoro, M. (2002). ZD6474, an orally available inhibitor of KDR tyrosine kinase activity, efficiently blocks oncogenic RET kinases. Cancer Res 62, 7284-7290.
Carlson, K. M., Bracamontes, J., Jackson, C. E., Clark, R., Lacroix, A., Wells, S. A., Jr., and Goodfellow, P. J. (1994a). Parent-of-origin effects in multiple endocrine neoplasia type 2B [see comments]. Am J Hum Genet 55,1076-1082.
Carlson, K. M., Dou, S., Chi, D., Scavarda, N., Toshima, K., Jackson, C. E., Wells, S. A., Jr., Goodfellow, P. J., and Donis-Keller, H. (1994b). Single missense mutation in the tyrosine kinase catalytic domain of the RET protooncogene is associated with multiple endocrine neoplasia type 2B. Proc Natl Acad Sci USA 91, 1579-1583.
Ciardiello, F., Bianco, R., Caputo, R., Damiano, V., Troiani, T., Melisi, D., De Vita, F., De Placido, S., Bianco, A. R., and Tortora, G. (2004). Antitumor activity of ZD6474, a vascular endothelial growth factor receptor tyrosine kinase inhibitor, in human cancer cells with acquired resistance to antiepidermal growth factor receptor therapy. Clin Cancer Res 10, 784-793.
Ciardiello, F., Caputo, R., Damiano, V., Troiani, T., Vitagliano, D., Carlomagno, F., Veneziani, B. M., Fontanini, G., Bianco, A. R., and Tortora, G. (2003). Antitumor effects of ZD6474, a small molecule vascular endothelial growth factor receptor tyrosine kinase inhibitor, with additional activity against epidermal growth factor receptor tyrosine kinase. Clin Cancer Res 9, 1546-1556.
Cirafici, A. M., Salvatore, G., De Vita, G., Carlomagno, F., Dathan, N. A., Visconti, R., Melillo, R. M., Fusco, A., and Santoro, M. (1997). Only the substitution of methionine 918 with a threonine and not with other residues activates RET transforming potential. Endocrinology 138, 1450-1455.
Donis-Keller, H., Dou, S., Chi, D., Carlson, K. M., Toshima, K., Lairmore, T. C., Howe, J. R., Moley, J. F., Goodfellow, P., and Wells, S. A., Jr. (1993). Mutations in the RET proto-oncogene are associated with MEN 2A and FMTC. Hum Mol Genet 2, 851-856.
Durick, K., Gill, G. N., and Taylor, S. S. (1998). Shc and Enigma are both required for mitogenic signaling by Ret/ptc2. Mol Cell Biol 18, 2298-2308.
Eng, C., Mulligan, L. M., Healey, C. S., Houghton, C., Frilling, A., Raue, F., Thomas, G. A., and Ponder, B. A. (1996). Heterogeneous mutation of the RET proto-oncogene in subpopulations of medullary thyroid carcinoma. Cancer Res 56, 2167-2170.
Eng, C., Smith, D. P., Mulligan, L. M., Nagai, M. A., Healey, C. S., Ponder, M. A., Gardner, E., Scheumann, G. F., Jackson, C. E., Tunnacliffe, A., and et al. (1994). Point mutation within the tyrosine kinase domain of the RET proto-oncogene in multiple endocrine neoplasia type 2B and related sporadic tumours [published erratum appears in Hum Mol Genet 1994 April; 3(4):686]. Hum Mol Genet 3, 237-241.
Glade-Bender, J., Kandel, J. J., and Yamashiro, D. J. (2003). VEGF blocking therapy in the treatment of cancer. Expert Opin Biol Ther 3, 263-276.
Hayashi, H., Ichihara, M., Iwashita, T., Murakami, H., Shimono, Y., Kawai, K., Kurokawa, K., Murakumo, Y., Imai, T., Funahashi, H., et al. (2000). Characterization of intracellular signals via tyrosine 1062 in RET activated by glial cell line-derived neurotrophic factor. Oncogene 19, 4469-4475.
Hennequin, L. F., Stokes, E. S., Thomas, A. P., Johnstone, C., Ple, P. A., Ogilvie, D. J., Dukes, M., Wedge, S. R., Kendrew, J., and Curwen, J. O. (2002). Novel 4-anilinoquinazolines with C-7 basic side chains: design and structure activity relationship of a series of potent, orally active, VEGF receptor tyrosine kinase inhibitors. J Med Chem 45, 1300-1312.
Hofstra, R. M., Landsvater, R. M., Ceccherihi, I., Stulp, R. P., Stelwagen, T., Luo, Y., Pasini, B., Hoppener, J. W., van Amstel, H. K., Romeo, G., and et al. (1994). A mutation in the RET proto-oncogene associated with multiple endocrine neoplasia type 2B and sporadic medullary thyroid carcinoma [see comments]. Nature 367, 375-376.
Kurokawa, K., Iwashita, T., Murakami, H., Hayashi, H., Kawai, K., and Takahashi, M. (2001). Identification of SNT/FRS2 docking site on RET receptor tyrosine kinase and its role for signal transduction. Oncogene 20, 1929-1938.
Lallier, M., St-Vil, D., Giroux, M., Huot, C., Gaboury, L., Oligny, L., and Desjardins, J. G. (1998). Prophylactic thyroidectomy for medullary thyroid carcinoma in gene carriers of MEN2 syndrome. J Pediatr Surg 33, 846-848.
Lips, C. J., Landsvater, R. M., Hoppener, J. W., Geerdink, R. A., Blijham, G., van Veen, J. M., van Gils, A. P., de Wit, M. J., Zewald, R. A., Berends, M. J., and et al. (1994). Clinical screening as compared with DNA analysis in families with multiple endocrine neoplasia type 2A [see comments]. N Engl J Med 331, 828-835.
Liu, X., Vega, Q. C., Decker, R. A., Pandey, A., Worby, C. A., and Dixon, J. E. (1996). Oncogenic RET receptors display different autophosphorylation sites and substrate binding specificities. J Biol Chem 271, 5309-5312.
Marshall, G. M., Peaston, A. E., Hocker, J. E., Smith, S. A., Hansford, L. M., Tobias, V., Norris, M. D., Haber, M., Smith, D. P., Lorenzo, M. J., et al. (1997). Expression of multiple endocrine neoplasia 2B RET in neuroblastoma cells alters cell adhesion in vitro, enhances metastatic behavior in vivo, and activates Jun kinase. Cancer Res 57, 5399-5405.
Melillo, R. M., Carlomagno, F., De Vita, G., Formisano, P., Vecchio, G., Fusco, A., Billaud, M., and Santoro, M. (2001a). The insulin receptor substrate (IRS)-1 recruits phosphatidylinositol 3-kinase to Ret: evidence for a competition between Shc and IRS-1 for the binding to Ret. Oncogene 20, 209-218.
Melillo, R. M., Santoro, M., Ong, S. H., Billaud, M., Fusco, A., Hadari, Y. R., Schlessinger, J., and Lax, I. (2001b). Docking protein FRS2 links the protein tyrosine kinase RET and its oncogenic forms with the mitogen-activated protein kinase signaling cascade. Mol Cell Biol 21, 4177-4187.
Menko, F. H., van der Luijt, R. B., de Valk, I. A., Toorians, A. W., Sepers, J. M., van Diest, P. J., and Lips, C. J. (2002). Atypical MEN type 2B associated with two germline RET mutations on the same allele not involving codon 918. J Clin Endocrinol Metab 87, 393-397.
Michiels, F. M., Chappuis, S., Caillou, B., Pasini, A., Talbot, M., Monier, R., Lenoir, G. M., Feunteun, J., and Billaud, M. (1997). Development of medullary thyroid carcinoma in transgenic mice expressing the RET protooncogene altered by a multiple endocrine neoplasia type 2A mutation. Proc Natl Acad Sci USA 94, 3330-3335.
Miller, M., Ginalski, K., Lesyng, B., Nakaigawa, N., Schmidt, L., and Zbar, B. (2001). Structural basis of oncogenic activation caused by point mutations in the kinase domain of the MET proto-oncogene: modeling studies. Proteins 44; 32-43.
Moley, J. F., DeBenedetti, M. K., Dilley, W. G., Tisell, L. E., and Wells, S. A. (1998). Surgical management of patients with persistent or recurrent medullary thyroid cancer. Journal of Internal Medicine 243, 521-526.
Moses, K., and Rubin, G. M. (1991). Glass encodes a site-specific DNA-binding protein that is regulated in response to positional signals in the developing Drosophila eye. Genes Dev 5, 583-593.
Mulligan, L. M., Eng, C., Healey, C. S., Clayton, D., Kwok, J. B., Gardner, E., Ponder, M. A., Frilling, A., Jackson, C. E., Lehnert, H., and et al. (1994). Specific mutations of the RET proto-oncogene are related to disease phenotype in MEN 2A and FMTC. Nat Genet 6, 70-74.
Mulligan, L. M., Kwok, J. B., Healey, C. S., Elsdon, M. J., Eng, C., Gardner, E., Love, D. R., Mole, S. E., Moore, J. K., Papi, L., and et al. (1993). Germ-line mutations of the RET proto-oncogene in multiple endocrine neoplasia type 2A. Nature 363, 458-460.
Ohiwa, M., Murakami, H., Iwashita, T., Asai, N., Iwata, Y., Imai, T., Funahashi, H., Takagi, H., and Takahashi, M. (1997). Characterization of Ret-Shc-Grb2 complex induced by GDNF, MEN 2A, and MEN 2B mutations. Biochem Biophys Res Commun 237, 747-751.
Pasini, A., Geneste, O., Legrand, P., Schlumberger, M., Rossel, M., Fournier, L., Rudkin, B. B., Schuffenecker, I., Lenoir, G. M., and Billaud, M. (1997). Oncogenic activation of RET by two distinct FMTC mutations affecting the tyrosine kinase domain. Oncogene 15, 393-402.
Pasini, B., Ceccherini, I., and Romeo, G. (1996). RET mutations in human disease. Trends Genet 12, 138-144.
Pelicci, G., Troglio, F., Bodini, A., Melillo, R. M., Pettirossi, V., Coda, L., De Giuseppe, A., Santoro, M., and Pelicci, P. G. (2002). The neuron-specific Rai (ShcC) adaptor protein inhibits apoptosis by coupling Ret to the phosphatidylinositol 3-kinase/Akt signaling pathway. Mol Cell Biol 22, 7351-7363.
Reynolds, L., Jones, K., Winton, D. J., Cranston, A., Houghton, C., Howard, L., Ponder, B. A., and Smith, D. P. (2001). C-cell and thyroid epithelial tumours and altered follicular development in transgenic mice expressing the long isoform of MEN 2A RET. Oncogene 20, 3986-3994.
Romeo, G., Ceccherini, I., Celli, J., Priolo, M., Betsos, N., Bonardi, G., Seri, M., Yin, L., Lerone, M., Jasonni, V., and Martucciello, G. (1998). Association of multiple endocrine neoplasia type 2 and Hirschsprung disease. J Intern Med 243, 515-520.
Santoro, M. M., Penengo, L., Minetto, M., Orecchia, S., Cilli, M., and Gaudino, G. (1998). Point mutations in the tyrosinekinase domain release the oncogenic and metastatic potential of the Ron receptor. Oncogene 17, 741-749.
Schuchardt, A., D'Agati, V., Larsson-Blomberg, L., Costantini, F., and Pachnis, V. (1994). Defects in the kidney and enteric nervous system of mice lacking the tyrosine kinase receptor Ret [see comments]. Nature 367, 380-383.
Smith-Hicks, C. L., Sizer, K. C., Powers, J. F., Tischler, A. S., and Costantini, F. (2000). C-cell hyperplasia, pheochromocytoma and sympathoadrenal malformation in a mouse model of multiple endocrine neoplasia type 2B. Embo J 19, 612-622.
Soler, R. M., Dolcet, X., Encinas, M., Egea, J., Bayascas, J. R., and Comella, J. X. (1999). Receptors of the glial cell line-derived neurotrophic factor family of neurotrophic factors signal cell survival through the phosphatidylinositol 3-kinase pathway in spinal cord motoneurons. J Neurosci 19, 9160-9169.
Songyang; Z., Carraway, K., Eck, M., Harrison, S., Feldman, R., Mohammadi, M., Schlessinger, J., Hubbard, S., Smith, D., Eng, C., et al. (1995). Catalytic specificity of protein-tyrosine kinases is critical for selective signalling. Nature 373, 536-539.
Takahashi, M. (1997). The role of the ret proto-oncogene in human disease. Nagoya J Med Sci 60, 23-30.
Takahashi, M.; Asai, N., Iwashita, T., Murakami, H., and Ito, S. (1998). Molecular mechanisms of development of multiple endocrine neoplasia 2 by RET mutations. J Intern Med 243, 509-513.
Takahashi, M., Ritz, J., and Cooper, G. (1985). Activation of a novel human transforming gene, ret, by DNA rearrangement. Cell 42, 581-588.
Tallini, G. (2002). Molecular pathobiology of thyroid neoplasms. Endocr Pathol 13, 271-288.
Tsuzuki, T., Takahashi, M., Asai, N., Iwashita, T., Matsuyama, M., and Asai, J. (1995). Spatial and temporal expression of the ret proto-oncogene product in embryonic, infant and adult rat tissues. Oncogene 10, 191-198.
Wedge, S. R., Ogilvie, D. J., Dukes, M., Kendrew, J., Chester, R., Jackson, J. A., Boffey, S. J., Valentine, P. J., Curwen, J. O., Musgrove, H. L., et al. (2002). ZD6474 inhibits vascular endothelial growth factor signaling, angiogenesis, and tumor growth following oral administration. Cancer Res 62, 4645-4655.
Wells, S. A. (1994). Genetic tests predict thyroid cancer risk, making preventive surgery possible. Journal of National Cancer Institute 86, 1268-1270.

What is claimed is:

1. A method for high throughput screening of compounds, the method comprising:
inducing a screenably distinct characteristic in wild-type Drosophila larvae by modifying expression of one or more Drosophila genes, said screenably distinct characteristic associated with human tumor metastasis;
exposing the Drosophila larvae to a compound that putatively modifies the screenably distinct characteristic; and
screening the Drosophila to determine whether the compound modifies the screenably distinct characteristic.

2. A method according to claim 1, wherein the screenably distinct characteristic comprises one of cell migration, apoptosis and abnormal tissue growth.

3. A method according to claim 1, wherein inducing a screenably distinct characteristic in wild-type Drosophila using targeted expression of one or more Drosophila genes comprises modifying expression of one or more of Csk, Src, cadherin, P120-catenin Rho1, Jnk, metalloprotease MMP2, or DIAP1.

4. A method according to claim 1, wherein modifying expression of one or more Drosophila genes comprises reducing or eliminating dCsk gene expression.

5. A method according to claim 4, further comprising spatially limiting the reduction or elimination of dCsk gene expression.

6. A method according to claim 5, comprising spatially limiting reduction or elimination of dCsk expression to the Drosophila developing eye.

7. A method according to claim 5, comprising further spatially limiting reduction or elimination of dCsk expression to a plurality of discrete clonal patches having boundary cells adjacent to wild-type cells.

8. A method according to claim 5, comprising spatially limiting reduction or elimination of dCsk expression to the Drosophila developing wing.

9. A method according to claim 4, comprising reducing or eliminating dCsk gene expression in a developing Drosophila using an RNA interference construct.

10. A method according to claim 1, further comprising screening the Drosophila to determine whether the compound has a toxic effect on the Drosophila.

11. An animal model of human tumor metastasis comprising:
a Drosophila having spatially limited modified expression of dCsk.

12. An animal model of human tumor metastasis according to claim 11, wherein dCsk expression is reduced in the Drosophila developing eye.

13. An animal model of human tumor metastasis according to claim 12, wherein reduced dCsk expression is spatially limited to the Drosophila developing eye.

14. An animal model of human tumor metastasis according to claim 13, wherein reduced dCsk expression is further spatially limited to a plurality of discrete clonal patches having boundary cells adjacent to wild-type cells.

15. An animal model of human tumor metastasis according to claim 11, wherein dCsk expression is spatially limited to the Drosophila developing wing.

16. An animal model of human tumor metastasis according to claim 15, wherein reduced dCsk expression is further spatially limited to a plurality of discrete clonal patches having boundary cells adjacent to wild-type cells.

17. A bioassay method for assaying the effects of a candidate anti-metastatic compound, said method comprising:
modifying at least one of cell migration, apoptosis and abnormal tissue growth in a Drosophila by modifying expression of dCsk; and
exposing the Drosophila to a compound that putatively modifies at least one of cell migration, apoptosis and abnormal tissue growth.

18. A bioassay method according to claim 17, wherein modifying expression of dCsk comprises spatially limiting the modified expression of dCsk.

19. A bioassay method according to claim 18, wherein spatially limiting the modified expression of dCsk comprises reducing dCsk expression and spatially limiting reduced dCsk expression to the Drosophila developing eye.

20. A bioassay method according to claim 18, wherein reduced dCsk expression is further spatially limited to a plurality of discrete clonal patches having boundary cells adjacent to wild-type cells.

21. A bioassay method according to claim 18, wherein spatially limiting the modified expression of dCsk comprises reducing dCsk expression and spatially limiting reduced dCsk expression to the Drosophila developing wing.

22. A bioassay method according to claim 21, wherein reduced dCsk expression is further spatially limited to a plurality of discrete clonal patches having boundary cells adjacent to wild-type cells.

23. A bioassay method comprising:
in a first Drosophila, reducing dCsk expression and spatially limiting reduced dCsk expression to the Drosophila developing eye; and
in a second Drosophila, reducing dCsk expression in the Drosophila developing eye and further spatially limiting reduced dCsk to a plurality of discrete clonal patches having boundary cells adjacent to wild-type cells;
exposing the first Drosophila and the second Drosophila to a compound that putatively modifies at least one of cell migration, apoptosis and abnormal tissue growth; and
comparing the effect of the compound on at least one of cell migration, apoptosis and abnormal tissue growth in the first Drosophila and the second Drosophila.

24. Apparatus for use in a high throughput screening assay method comprising:
a multi-well microtiter plate;
an amount of a Drosophila growth medium placed into said multiple wells of said multi-well microtiter plate;
an amount of a candidate compound added to said multiple wells; and
at least one Drosophila in each of said multiple wells, said Drosophila having modified dCsk expression and expression of at least one screenably distinct characteristic.

25. Apparatus according to claim 24, wherein said Drosophila with modified expression of dCsk comprises a Drosophila with reduced or eliminated expression of dCsk gene.

26. Apparatus according to claim 24, wherein said Drosophila having expression of at least one screenably distinct characteristic comprises a characteristic associated with human tumor metastasis.

27. Apparatus according to claim 26, wherein the at least one screenably distinct characteristic is selected from the group consisting of cell migration, apoptosis and abnormal tissue growth.

28. Apparatus according to claim 26, wherein the screenably distinct characteristic comprises abnormal tissue growth in the wing.

29. Apparatus according to claim 26, wherein the screenably distinct characteristic comprises abnormal tissue growth in the eye.
